(19) United States
(12) Patent Application Publication
(10) Pub. No.: US 2021/0290538 A1
(43) Pub. Date: Sep. 23, 2021

(54) MILK VESICLES FOR USE IN DELIVERING BIOLOGICAL AGENTS

(71) Applicant: PureTech LYT, Inc., Boston, MA (US)

(72) Inventors: Joseph Bolen, Boston, MA (US); Rishab Shyam, Boston, MA (US); Nicholas Pilla, Boston, MA (US); Katerina Krumova, Boston, MA (US); Bhushan Pattni, Boston, MA (US); Daniel Bonner, Boston, MA (US)

(73) Assignee: PureTech LYT, Inc., Boston, MA (US)

(21) Appl. No.: 17/257,452

(22) PCT Filed: Jul. 2, 2019
(86) PCT No.: PCT/US19/40425

§ 371 Date: Dec. 31, 2020

Related U.S. Application Data
(60) Provisional application No. 62/693,115, filed on Jul. 2, 2018.

(51) Int. Cl. A61K 9/127; A61K 35/20; A61K 31/7088; A61K 47/42

(52) U.S. Cl. CPC A61K 9/1276; A61K 47/42; A61K 31/7088; A61K 35/20

(57) ABSTRACT

The present disclosure provides milk vesicles as drug delivery vehicles, compositions comprising a therapeutic agent encapsulated within or otherwise associated with the milk vesicles, methods of producing such milk vesicles and compositions thereof, as well as methods of delivering such milk vesicles and compositions to a specific patient tissue or organ. Also provided herein is a composition comprising milk vesicles, wherein the milk vesicles comprise a lipid membrane to which one or more proteins are associated, and wherein (a) a relative abundance of casein in the composition is less than about 40%, and/or (b) a relative abundance of lactoglobulin in the composition is less than about 25%.

### CROSS REFERENCE TO RELATED APPLICATIONS

This application claims the benefit of the filing dates of U.S. Provisional Application No. 62/693,115, filed Jul. 2, 2018, the entire contents of which is incorporated by reference herein.

### TECHNICAL FIELD

The present disclosure relates, at least in part, to vesicles found in milk, which vesicles are capable of carrying, e.g., in association with a membrane, or loading (e.g., encapsulating, covalent or non-covalent attachment to the vesicle membrane, integral vesicle proteins, membrane lipids or oligosaccharides), biological agents, for example, small molecules and biologics, such as proteins, peptides, nucleic acids, or other agents, and, in some embodiments, improving their stability or other properties and/or delivering them to a tissue or organ in a patient. The present disclosure also relates to compositions and methods of using such milk vesicles.

### BACKGROUND OF THE INVENTION

Recent years have seen tremendous development of biologics and related therapeutic agents to treat, diagnose, and monitor disease. However, the challenge of generating suitable vehicles to package, stabilize and deliver payloads to sites of interest remains unaddressed. Many therapeutics suffer from degradation due to their inherent instability and active clearance mechanisms in vivo. Poor in vivo stability is particularly problematic when delivering these payloads orally. The harsh conditions of the digestive tract, including acidic conditions in the stomach, peristaltic motions coupled with the action of proteases, lipases, amylases, and nucleases that break down ingested components in the gastrointestinal tract, make it particularly challenging to deliver many biologics orally. The scale of this challenge is evidenced by the number of biologics limited to delivery via non-oral means, including IV, transdermal, and sub-cutaneous administration. A general oral delivery vehicle for biologics and related therapeutic agents would profoundly impact healthcare.

Recent efforts have focused on the packaging of biologics into polymer-based, liposomal, or biodegradable and erodible matrices that result in biologic-encapsulated nanoparticles. Despite their advantageous encapsulation properties, such nanoparticles have not achieved widespread use due to toxicity or poor release properties. Additionally, current nanoparticle synthesis techniques are limited in their ability to scale for manufacturing purposes. The development of an effective, non-toxic, and scalable delivery platform thus remains an unmet need.

Milk, which is orally ingested and known to contain a variety of miRNAs important for immune development, protects and delivers these miRNAs in exosomes. Milk vesicles therefore represent a gastrointestinally-privileged (GI-privileged), evolutionarily conserved means of communicating important messages from mother to baby while maintaining the integrity of these complex biomolecules. As one example, milk exosomes have been observed to have a favorable stability profile at acidic pH and other high-stress or degradative conditions (See, e.g., Int J Biol Sci. 2012; 8(1):118-23. Epub 2011 Nov. 29). Additionally, bovine miRNA levels in circulation have been observed to increase in a dose-dependent manner after consuming varying quantities of milk (See, e.g., PLoS One 2015; 10(3): e0121123).

Collectively, the available data suggest that humans have the ability to absorb intact nucleic acid contents of milk.

### SUMMARY OF THE INVENTION

Milk vesicles, for example milk exosomes and other vesicles, which can encapsulate or otherwise carry miRNA species can enable oral delivery of a variety of therapeutic agents. The present disclosure harnesses milk-derived vesicles to meet the urgent need for suitable delivery vehicles for therapeutics that were previously not orally administrable or suffered from other delivery challenges such as poor bioavailability, storage instability, metabolism, off-target toxicity, or decomposition in vivo.

Accordingly, one aspect of the present disclosure features a cargo-loaded milk vesicle, wherein the milk vesicle comprises a lipid membrane to which one or more proteins are associated, and wherein the milk vesicle is loaded with a cargo, which is an exogenously added biological molecule. In some embodiments, the biological molecule is a molecule that is not naturally-occurring in the milk vesicle. In some embodiments, the size of the milk vesicle is about 20-1,000 nm, for example, about 80-200 nm or about 120-160 nm. Particle size can be determined by nanoparticle tracking analysis (NTA) or dynamic light scattering (DLS), or microfluidic resistive pulse sensing.

In some embodiments, the cargo-loaded milk vesicle described herein may comprise one or more proteins selected from Butyrophilin Subfamily 1 Member A1 (BTN1A1) or a transmembrane fragment thereof, Butyrophilin Subfamily 1 Member A2 (BTN1A2) or a transmembrane fragment thereof, fatty acid binding protein, lactadherin, β-lactoglobulin, platelet glycoprotein 4, xanthine dehydrogenase, ATP-binding cassette subfamily G, perillipin, platelet glycoprotein 4, RAB1A, peptidyl-prolyl cis-transisomerase A, Ras-related protein Rab-18, EpCAM, CD63, CD81, TSG101, HSP70, lactoferrin or a transmembrane fragment thereof, ALG-2-interacting protein X, kappa-casein, alpha-lactalbumin, serum albumin, alpha-S1-casein, alpha-S2-casein, polymeric immunoglobulin, lactoperoxidase, or a combination thereof. In one particular example, the cargo-loaded milk vesicle comprises BTN1A1, BTN1A2, or a combination thereof. One or more of the protein moieties in the cargo-loaded milk vesicle may be glycosylated. In some non-limiting examples, the glycosylated proteins comprise terminal β-galactose, terminal α-galactose, N-acetyl-D-galactosamine, and/or N-acetyl-D-glycosamine.

Alternatively or in addition, the lipids of the cargo-loaded milk vesicle described herein may comprise hexosylceramide (HexCer), ganglioside GM1, ganglioside GM2, ganglioside GM3, ganglioside GD1, lactosylceramide (LacCer), sphingomyelin (SM), L-alpha-lysophosphatidylinositol (LPI), cholesterol (CHOL), phosphatidylserine (PS), globotriaosylceramide (Gb3), phosphatidic acid (PA), diacylglycerol (DAG), ceramide (Cer), or a combination thereof.

In another aspect, the present disclosure provides a composition comprising milk vesicles, wherein the milk vesicles comprise a lipid membrane to which one or more proteins are associated, and wherein the relative abundance of casein in the composition is less than about 40% (e.g., less than about 35%, about 30%, about 25%, about 20%, about 15%, about 10%, about 5% or less, including any numerical increment between these listed ranges). In another aspect, the present disclosure provides a composition comprising milk vesicles, wherein the milk vesicles comprise a lipid membrane to which one or more proteins are associated, and wherein the relative abundance of lactoglobulin in the composition is less than about 25% (e.g., less than about 20%, about 15%, about 10% or less, including any numerical increment between these listed ranges). In another aspect, the present disclosure provides a composition comprising milk vesicles, wherein the milk vesicles comprise a lipid membrane to which one or more proteins are associated, and wherein the relative abundance of casein in the composition is less than about 40% (e.g., less than about 35%, about 30%, about 25%, about 20%, about 15%, about 10%, about 5% or less, including any numerical increment between these listed ranges) and the relative abundance of lactoglobulin in the composition is less than about 25% (e.g., less than about 20%, about 15%, about 10% or less, including any numerical increment between these listed ranges). As used herein, the term “relative abundance” refers to the percentage of casein or lactoglobulin in the total protein content of the composition. In some examples, the composition comprising the milk vesicles is substantially free of caseins and/or lactoglobulins. The casein and/or lactoglobulins may be removed from the milk vesicles in accordance with methods disclosed herein.

In some embodiments, the present disclosure provides milk vesicles that have been isolated and/or purified from milk and/or milk products and/or milk components using any of the methods and milk sources provided herein. In some embodiments, the milk vesicles are modified from their naturally-occurring milk vesicle counterparts. In some embodiments, the lipid membrane of the milk vesicle has been modified from the lipid membrane of its naturally-occurring milk vesicle counterpart. In some embodiments, the milk vesicle has been modified from its naturally-occurring milk vesicle counterpart by the inclusion (addition) or exclusion (removal) of one or more of the following molecules: lipid, phospholipid, glycolipid, protein, peptide, glycoprotein, phosphoprotein, glycan, glyceride, fatty acid and any of the other molecules disclosed elsewhere herein. In some embodiments, the milk vesicle is modified such that it contains less casein(s) and/or lactoglobulin(s) than its naturally-occurring counterpart. In some examples, the milk vesicle is modified such that it is substantially free of caseins and/or lactoglobulins. In any of these embodiments of modified milk vesicles, the milk vesicle may or may not be further modified to contain cargo. In some embodiments, the modified milk vesicle does not contain cargo. In some embodiments, the milk vesicle contains cargo.

In some embodiments, the milk vesicles have been modified from their naturally-occurring milk vesicle counterparts by being loaded with cargo. In some embodiments in which the milk vesicle has been modified to contain cargo, the milk vesicle into which the cargo is loaded may be a naturally-occurring milk vesicle. In some embodiments in which the milk vesicle has been modified to contain cargo, the milk vesicle into which the cargo is loaded may be a milk vesicle that has been modified from its naturally-occurring milk vesicle counterpart, such as any of the modified milk vesicles described in the above pargarphs and elsewhere herein.

In some embodiments, the milk vesicles in the composition are loaded with a cargo, which is an exogenoulsy added biological molecule. In some embodiments, the biological molecule is a molecule that is not naturally-occurring in the milk vesicle. Examples of various different cargos are provided herein. The milk vesicles may have the size ranges as disclosed herein. In some embodiments, the one or more proteins (e.g., glycoproteins) associated with the lipid membrane of the milk vesicles comprise Butyrophilin Subfamily 1 Member A1 (BTN1A1) or a transmembrane fragment thereof, Butyrophilin Subfamily 1 Member A2 (BTN1A2) or a transmembrane fragment thereof, fatty acid binding protein, lactadherin, platelet glycoprotein 4, xanthine dehydrogenase, ATP-binding cassette subfamily G, perillipin, RAB1A, peptidyl-prolyl cis-transisomerase A, Ras-related protein Rab-18, EpCAM, CD63, CD81, TSG101, HSP70, lactoferrin or a transmembrane fragment thereof, ALG-2-interacting protein X, alpha-lactalbumin, serum albumin, polymeric immunoglobulin, lactoperoxidase, or a combination thereof. In some examples, the milk vesicles comprise BTN1A1 and CD81. In some embodiments, cargo-loaded milk vesicles comprise a lipid membrane wherein the relative abundance of casein is less than about 40% and/or the relative abundance of lactoglobulin is less than about 25%. In some embodiments, the cargo-loaded milk vesicles comprise a lipid membrane which is substantially free of caseins and/or lactoglobulins.

In some embodiments, the milk vesicles disclosed herein may comprise one or more of the following features:

(a) stablility under freeze-thaw cycles and/or temperature treatment;

(b) colloidal stablility when the milk vesicles are loaded with the biological molecule;

(c) a loading capacity of at least 1000 (e.g., at least 2000, at least 3000, at least 4000, or at least 5000) cholesterol modified oligonucleotides per milk vesicle;

(d) stability under acidic pH (e.g., ≤4.5; or ≤2.5

(e) stability upon sonication;

(f) resistance to enzyme digestion (e.g., to one or more digestive enzymes);

(g) resistance to nuclease treatment upon loading of the milk vesicles with oligonucleotides; and

(h) resistance to protease treatment upon loading of the milk vesicles with peptides or proteins.

In some examples, the enzyme digestion comprises digestion by one or more digestive enzymes, e.g., proteases, lipases, amylases, and/or nucleases. Non-limiting examples include lingual lipase, salivary amylase, pepsin, gastric lipase, trypsin, chymotrypsin, cardoxypeptidase, elastase, pancreatic lipase, phospholipase, DNAase, RNAase, pancreatic amylase, erepsin, maltase, lactase, and/or sucrose.

The milk vesicles described herein may be obtained from a suitable mammal, for example, cow milk, goat milk, camel milk, buffalo milk, yak milk, or human milk. In some embodiments, milk vesicle is obtained from raw milk, skim milk, colostrum, homogenized milk, pasteurized milk, acidified milk, or whey. Exemplary methods for isolating the milk vesicle described herein include, but are not limited to, differential ultracentrifugation, size exclusion chromatography, affinity purification, tangential flow filtration, or a combination thereof.

In some embodiments, the milk vesicle is a lactosome, a milk fat globule (MFG), an exosome, an extracellular vesicle, a whey-particle, a whey-derived particle, or an aggregate thereof, or a combination of such globules, vesicles, and/or particles.

In any of the cargo-loaded milk vesicles described herein, the cargo is a biological molecule, e.g., a peptide, a protein, a nucleic acid, a polysaccharide, or a small molecule. Non-limiting exemplary proteins, polypeptides, and/or peptides include an antibody, a hormone, a growth factor, an enzyme, a cytokine, a chemokine, a toxin, an antitoxin, a blood coagulation factor, or a combination thereof. Non-limiting exemplary nucleic acid molecules include an interfering RNA (iRNA), a microRNA (miRNA), an antisense RNA, a messenger RNA (mRNA), a non-coding RNA, a single-stranded DNA (ssDNA), a double-stranded DNA (dsDNA), or a combination thereof. Specific iRNA includes siRNA or shRNA. Any of the nucleotide molecules disclosed herein, or a fragment thereof, may comprise a naturally-occurring nucleotide sequence. Alternatively, the nucleotide molecules can be synthetic (non-naturally occurring). In some embodiments, the cargo is a biological molecule that is not naturally-occurring in the milk vesicle. In some embodiments, the cargo is a biological molecule that may be endogenous to the milk vesicle but is added exogenously.

In some embodiments, the biological molecule can be conjugated to a hydrophobic moiety. Non-limiting examples include a lipid, a sterol, a steroid, a terpene, cholic acid, adamantine acetic acid, 1-pyrene butyric acid, 1,3-bis-O(hexadecyl)glycerol, a geranyloxyhexyl group, hexadecylglycerol, borneol, 1,3-propanediol, heptadecyl group, O3-(oleoyl)lithocholid acid, O3-(oleoyl)cholenic acid, dimethoxytrityl, phenoxazine, isoprene derivatives (e.g., solanesol, farnesol, ubiquinol, geranol etc), tocopherol, or tocotrienols.

In another aspect, provided herein are pharmaceutical compositions comprising any of the milk vesicles described herein, including modified or naturally-occurring milk vesicles and a pharmaceutically acceptable carrier. In another aspect, provided herein are pharmaceutical compositions comprising any of the milk vesicles described herein, including modified or naturally-occurring milk vesicles which may be cargo-loaded, and a pharmaceutically acceptable carrier. In some embodiments, the pharmaceutical composition can be formulated for oral administration.

In yet another embodiment, the present disclosure provides a method of delivering orally a biological molecule to a subject, the method comprising administering orally to a subject in need thereof a cargo-loaded milk vesicle as described herein, or a pharmaceutical composition comprising such. In some embodiments, the subject is a human patient having, suspected of having, or at risk for a target disease, for example, a hyperproliferative disease, an infectious disease, an autoimmune disease, an inflammatory disease, an allergic disease, a cardiovascular disease, a metabolic disease, or a neurodegenerative disease. The subject may have been treated or is undergoing an additional treatment for the target disease.

Further, the present disclosure provides a method for preparing a cargo-loaded milk vesicle, comprising contacting a milk vesicle with a biological molecule (e.g., those described herein) under conditions allowing for loading of the biological molecule into the milk vesicle.

In some embodiments, the method for preparing a composition comprising milk vesicles may comprise: (i) providing a first milk sample; (ii) removing casein and/or lactoglobulin from the first milk sample to produce a second milk sample; and (iii) isolating milk vesicles from the second milk sample to produce a composition comprising the milk vesicles. In some examples, the first milk sample can be from cow milk, goat milk, camel milk, buffalo milk, yak milk, or human milk. Alternatively or in addition, the first milk sample is raw milk, skim milk, colostrum, homogenized milk, whey, or pasteurized milk.

In some embodiments, the removing step (ii) in any of the methods disclosed herein can be performed by acidifying the first milk sample. Alternatively, the removing step (ii) is performed by coagulating the first milk sample with rennet, e.g., animal rennet such as rennet derived from calf intestine, or plant rennet such as vegetable rennet. In other examples, the removing step (ii) can be performed by disrupting casein micelles by EDTA, EGTA, or another Ca²⁺ chelating agent.

Alternatively or in addition, the isolating step (iii) of any of the methods disclosed herein can be performed by ultracentrifugation, size exclusion chromatography, affinity purification, tangential flow filtration, or a combination thereof.

The details of one or more embodiments of the invention are set forth in the description below. Other features or advantages of the present invention will be apparent from the following drawings and detailed description of several embodiments, and also from the appended claims.

### BRIEF DESCRIPTION OF THE FIGURES

FIG. 1A is a diagram illustrating an exemplary process for isolating extracellular vesicles from milk and colostrum powder by ultracentrifugation.

FIG. 1B is a chart showing protein concentration of exosomes isolated from skim milk by size exclusion chromatography.

FIGS. 2A-2B include charts showing representative proteins identified in acidified skim milk (cow) exosomes (2A) and in goat exosomes (2B).

FIGS. 3A-3B are photos showing protein stability within exosomes in different fluid systems by SDS-PAGE analysis. FIG. 1A: exosomes from colostrum milk, raw milk, and skim milk that had been incubated with simulated gastric fluid (SGF) for 0, 1, or 4 hours. FIG. 1B: exosomes from colostrum milk, raw milk, and skim milk that had been incubated with simulated intestinal fluid (SIF) for 0, 1, or 4 hours.

FIG. 4 is a chart showing particle concentrations (particles/mL) of exosomes from reconstituted colostrum powder (CM), raw milk (RM), skim milk (SM), and goat milk (GM) after being incubated with SGF (pH ˜4.5) and SIF (pH ˜7) for various time periods as indicated.

FIGS. 5A-5D include charts showing particle sizes and particle concentrations of exosomes incubated with SGF or SIF for various periods. 5A: particle size of exosomes or acidified exosomes prepared by ultracentrifugation (UC), wherein the exosomes have been incubated in SGF (pH 2) for the time periods as indicated. 5B: particle size of exosomes or acidified exosomes prepared by ultracentrifugation (UC), wherein the exosomes have been incubated in SGF (pH 5) for the time periods as indicated. 5C: particle concentration of exosomes or acidified exosomes prepared by ultracentrifugation (UC), wherein the exosomes have been incubated in SGF (pH 2) for the time periods as indicated. 5D: particle concentration of exosomes or acidified exosomes prepared by ultracentrifugation (UC), wherein the exosomes have been incubated in SGF (pH 5) for the time periods as indicated.

FIGS. 6A-6D include charts showing particle concentrations and particle sizes of exosomes from different milk sources after being incubated in SGF or SIF, or under the chemical and physical conditions as indicated. 6A: particle concentration of raw milk exosomes incubated under the conditions as indicated for the time periods as also indicated. 6B: particle size of raw milk exosomes incubated under the conditions as indicated for the time periods as also indicated. 6C: particle concentration of skim milk exosomes incubated under the conditions as indicated for the time periods as also indicated. 6D: particle size of skim milk exosomes incubated under the conditions as indicated for the time periods as also indicated. Results were obtained from nanoparticle tracking analysis (NTA).

FIGS. 7A-7C include diagrams showing protein content of milk vesicle compositions prepared by methods involving casein removal. FIG. 7A: a photo showing SDS-PAGE and Coomassie staining of protein contents of milk vesicle compositions prepared by casein removal by acidification and filtration or low-speed centrifugation followed by tangential flow filtration (ATFF), casein removal and filtration or low-speed centrifugation by acidification followed by tangential flow filtration and size exclusion chromatography (ATFF/SEC), Ultracentrifugation (UC), Disruption of casein micelles by EDTA followed by ultracentrifugation (EUC), and casein removal by acidification and filtration or low-speed centrifugation followed by ultracentrifugation (AUC). FIG. 7B: a chart showing relative abundance of casein band (25-30 kDa) in milk exosomes (extracellular vesicles) (MEV) isolation. FIG. 7C: a chart showing relative abundance of low molecule weight bands (lactoglobulin enriched fraction) in MEV isolation.

FIGS. 8A-8B include diagrams showing protein content of milk vesicle compositions prepared using vegetable rennet. FIG. 8A: a photo showing SDS-PAGE and Coomassie staining of protein contents of milk vesicle compositions prepared by ATFF/SEC and casein removal by coagulation with vegetable rennet and mechanical removal or filtration or low-speed centrifugation followed by tangential flow filtration and size exclusion chromatography (VR-TFF/SEC). FIG. 8B: a chart showing milk vesicle yields by various batches of the VRTFF/SEC approach and the ATCC/SEC approach.

FIG. 9 is a photo showing co-presence of CD81 and BTN1A1 on milk vesicles using co-immunoprecipitation followed by western blot.

FIG. 10A-10F include diagrams showing tolerance of milk vesicles to freeze-thaw cycles and temperature treatment. FIG. 10A: a chart showing particle concentration of milk vesicle compositions after five cycles of freeze-thaw (FTC) or after temperature treatment (4° C. for 24 hours, 60° C. for 40 minutes, or 100° C. for 10 minutes). FIG. 10B: a chart showing particle size of milk vesicle compositions after five cycles of freeze-thaw (FTC) or after temperature treatment (4° C. for 24 hours, 60° C. for 40 minutes, or 100° C. for 10 minutes). FIG. 10C: a photo showing protein content of milk vesicle compositions after five cycles of freeze-thaw (FTC) or after temperature treatment (4° C. for 24 hours, 60° C. for 40 minutes, or 100° C. for 10 minutes) as determined by SDS/PAGE. FIGS. 10D-10F: photos showing milk vesicle markers CD81 (FIG. 10D), CD9 (FIG. 10E), and BTN1A1 (FIG. 10F) of milk vesicle compositions after 6 cycles of freeze-thaw or after temperature treatment (4° C. for 24 hours, room temperature for 96 hours, 60° C. for 40 minutes, or 100° C. for 10 minutes).

FIGS. 11A-11D include charts showing that removal of casein does not affect milk vesicle stability in simulated gastric fluid (SGF). FIGS. 11A and 11B: charts showing mode particle size and particle concentration of milk vesicles prepared by UC and AUC incubated in SGF at pH 2, respectively. FIGS. 11C and 11D: charts showing mode particle size and particle concentration of milk vesicles prepared by UC and AUC incubated in SGF at pH 5, respectively.

FIG. 12 is a chart showing capacity of loading cholesterol modified oligonucleotides per milk vesicle prepared via ATFF/SEC.

FIGS. 13A and 13B include diagrams showing that milk vesicles protect oligonucleotides loaded therein from S1 nuclease digestion. FIG. 13A: a diagram illustrating a process for testing protection of oligonucleotides from S1 nuclease by milk vesicles. MBCD refers to methyl beta cyclodextrin. FIG. 13B is a photo showing oligonucleotide fractions before and after S1 nuclease digestion.

FIGS. 14A-14B include photos showing protection of oligonucleotides by milk vesicles. FIG. 14A: a photo showing rennet does not affect milk vesicle protection properties of S1 nuclease digestion of oligonucleotides loaded into the milk vesicle. FIG. 14B: a photo showing protection of antisense oligonucleotides (ASO) loaded into milk vesicles prepared by VR-TFF/SEC.

FIGS. 15A-15C include photos showing that, unlike milk vesicles, PEGylated liposomes do not protect cholesterol-modified oligonucleotides from S1 nuclease. FIG. 15A: a photo showing protection properties of milk vesicles as compared with PEGylated liposomes. FIG. 15B: a photo showing that calcium/ethanol precipitation of oligonucleotides does not lead to efficient protection from S1 nuclease. FIG. 15C: a photo showing protection of cholesterol modified oligonucleotide in presence of milk exosome.

### DETAILED DESCRIPTION OF THE INVENTION

Disclosed herein are milk vesicles or vehicles loaded with cargos (cargo-loaded milk vesicles or vehicles), which are biological molecules that have been exogenosly added or loaded to such milk vesicles or vehicles, pharmaceutical compositions comprising such, method of using the milk vehicles for delivering (e.g., orally) biological molecules to a subject in need thereof, as well as methods for making the cargo-loaded milk vehicles. Milk vesicles and milk vehicles are used herein interchangeably.

### I. Milk Vesicles

Milk vesicles, as used herein, refer to any particles found in milk of any suitable mammal source (e.g., those described herein). Milk vesicles, including microvesicles, typically are in the form of small assemblies of lipids about 20 to 1000 nm in size. The lipids in milk vehicles often form membrane structures, to which one or proteins are associated (e.g., attached to the surface of the lipid membrane and/or embedded inside the lipid membrane).

Milk vesicles, for example milk exosomes and other vesicles, which can encapsulate or otherwise carry miRNA species, can enable oral delivery of a variety of therapeutic agents. The present disclosure harnesses milk-derived vesicles to meet the urgent need for suitable delivery vehicles for therapeutics that were previously not orally administrable or suffered from other delivery challenges such as poor bioavailability, storage instability, metabolism, off-target toxicity, or decomposition in vivo. In one aspect, the present invention provides vesicles derived from milk, as vehicles for therapeutic agents such as DNA, RNA, iRNA, mRNA, siRNA, antisense oligonucleotides, analogs of nucleic acids, antibodies, hormones, and other peptides and proteins. In one aspect, the present invention provides vesicles derived from milk as vehicles for diagnostics or imaging agents.

In some embodiments, the milk vesicle is approximately round or spherical in shape. In some embodiments, the milk vesicle is approximately ovoid, cylindrical, tubular, cube, cuboid, ellipsoid, or polyhedron in shape. In some embodiments, the milk vesicle may be part of a cluster, collection, or formation of milk vesicles.

In some embodiments, the milk vesicle is able to transport one or more agents, e.g., therapeutic agent, through a mammalian gut such that the agent has systemic and/or tissue bioavailability. In some embodiments, the milk vesicle is able to deliver one or more agents, e.g., therapeutic agent, to one or more mammalian tissue(s).

Also provided herein are compositions comprising milk vesicles as disclosed herein, wherein the compositions have a relative abundance of proteins with a molecular weight of about 25-30 kDa (e.g., casein) no greater than about 40% and/or a relative abundance of proteins with a molecular weight of about 10-20 kDa (e.g., lactoglobulin) no greater than 25%.

A. Size of Milk Vesicles

In some descriptions, e.g., where diameter is a relevant measurement, such as in spherical and other shaped vesicles having a measurable diameter, the terms “size” and “diameter” are used interchangeably. The milk vesicle can be about 20 nm-1000 nm in diameter or size. In some embodiments, the milk vesicle is about 20 nm to about 200 nm in size. In some embodiments, the milk vesicle is about 20 nm to about 190 nm or about 25 nm to about 190 nm in size. In some embodiments, the milk vesicle is about 30 nm to about 180 nm in size. In some embodiments, the milk vesicle is about 35 nm to about 170 nm in size. In some embodiments, the milk vesicle is about 40 nm to about 160 nm in size. In some embodiments, the milk vesicle is about 50 nm to about 150 nm, about 60 nm to about 140 nm, about 70 nm to about 130 nm, about 80 nm to about 120 nm, or about 90 nm to about 110 nm in size. In some embodiments, the milk vesicle is about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm in size or diameter. In some embodiments, an average vesicle size in a vesicle composition or plurality of vesicles isolated or derived from milk is about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm in average size. In some embodiments, an average vesicle size in a vesicle composition or plurality of vesicles isolated or derived from milk is about 20 nm to about 200 nm, about 20 nm to about 190 nm, about 25 nm to about 190 nm, about 30 nm to about 180 nm, about 35 nm to about 170 nm, about 40 nm to about 160 nm, about 50 nm to about 150, about 60 to about 140 nm, about 70 to about 130, about 80 to about 120, or about 90 to about 110 nm in average size.

In some embodiments, the milk vesicle is about 20 nm to about 100 nm in size. In some embodiments, the milk vesicle is about 25 nm to about 95 nm in size. In some embodiments, the milk vesicle is about 20 nm to about 90 nm in size. In some embodiments, the milk vesicle is about 20 nm to about 85 nm in size. In some embodiments, the milk vesicle is about 20 nm to about 80 nm in size. In some embodiments, the milk vesicle is about 20 nm to about 75 nm in size. In some embodiments, the milk vesicle is about 20 nm to about 70 nm in size. In some embodiments, the milk vesicle is about 25 nm to about 80 nm in size. In some embodiments, the milk vesicle is about 30 nm to about 70 nm in size. In some embodiments, the milk vesicle is about 30 nm to about 60 nm in size. In some embodiments, the milk vesicle is about 40 nm to about 70 nm in size. In some embodiments, the milk vesicle is about 40 nm to about 60 nm in size. In some embodiments, an average vesicle size in a vesicle composition or plurality of vesicles isolated or derived from milk is about 20 nm to about 100 nm, about 20 nm to about 95 nm, about 20 nm to about 90 nm, about 20 nm to about 85 nm, about 20 nm to about 80 nm, about 20 to about 75 nm, about 25 nm to about 85 nm, about 25 nm to about 80, about 25 to about 75 nm, about 30 to about 80 nm, about 30 to about 85 nm, about 30 to about 75 nm, about 40 to about 80, about 40 to about 85 nm, about 40 to about 75 nm, about 45 to about 80 nm, about 45 to about 85, about 45 to about 75 nm, about 50 to about 75 nm, about 50 to about 80 nm, about 50 to about 85 nm, about 55 to about 75 nm, about 55 to about 80 nm, about 55 to about 85 nm, about 60 to about 75 nm, about 60 to about 80 nm, about 60 to about 85 nm, about 25 to about 70 nm, about 30 to about 70 nm, about 40 to about 70 nm, about 50 to about 70 nm, about 30 to about 60 nm, about 30 to about 50 nm in average size.

In some embodiments, the milk vesicle is about 80 nm to about 200 nm in size. In some embodiments, the milk vesicle is about 85 nm to about 195 nm in size. In some embodiments, the milk vesicle is about 90 nm to about 190 nm in size. In some embodiments, the milk vesicle is about 95 nm to about 185 nm in size. In some embodiments, the milk vesicle is about 100 nm to about 180 nm in size. In some embodiments, the milk vesicle is about 105 nm to about 175 nm in size. In some embodiments, the milk vesicle is about 110 nm to about 170 nm in size. In some embodiments, the milk vesicle is about 115 nm to about 165 nm in size. In some embodiments, the milk vesicle is about 120 nm to about 160 nm in size. In some embodiments, the milk vesicle is about 125 nm to about 155 nm in size. In some embodiments, the milk vesicle is about 130 nm to about 150 nm in size. In some embodiments, the milk vesicle is about 135 nm to about 145 nm in size. In some embodiments, an average vesicle size in a vesicle composition or plurality of vesicles isolated or derived from milk is about 80 nm to about 200 nm, about 80 nm to about 190 nm, about 80 nm to about 180 nm, about 80 nm to about 170 nm, about 80 nm to about 160 nm, about 80 to about 150 nm, about 80 nm to about 140 nm, about 80 nm to about 130, about 80 to about 120 nm, about 80 to about 110 nm, about 80 to about 100 nm, about 30 to about 75 nm, about 40 to about 80, about 40 to about 85 nm, about 40 to about 75 nm, about 45 to about 80 nm, about 45 to about 85, about 45 to about 75 nm, about 50 to about 75 nm, about 50 to about 80 nm, about 50 to about 85 nm, about 55 to about 75 nm, about 55 to about 80 nm, about 55 to about 85 nm, about 60 to about 75 nm, about 60 to about 80 nm, about 60 to about 85 nm, about 25 to about 70, about 30 to about 70, about 40 to about 70 nm, about 50 to about 70 nm, about 30 to about 60 nm, about 30 to about 50 nm in average size. In some embodiments, the milk vesicle is greater than 200 nm in size. In some embodiments, the milk vesicle is about 200 to about 1000 nm in size. In some embodiments, the milk vesicle is about 200 to about 400 nm in size, e.g., about 200 nm to about 250 nm, about 250 nm to about 300 nm, about 300 to about 350 nm, about 350 nm to about 400 nm in size. In some embodiments, the milk vesicle is about 400 to about 600 nm in size, e.g., about 400 nm to about 450 nm, about 450 nm to about 500 nm, about 500 to about 550 nm, about 550 nm to about 600 nm in size. In some embodiments, the milk vesicle is about 600 to about 800 nm in size, e.g., about 600 nm to about 650 nm, about 650 nm to about 700 nm, about 700 to about 750 nm, about 750 nm to about 800 nm in size. In some embodiments, the milk vesicle is about 800 to about 1000 nm in size, e.g., about 800 nm to about 850 nm, about 850 nm to about 900 nm, about 900 to about 950 nm, about 950 nm to about 1000 nm in size. In some embodiments, an average vesicle size in a vesicle composition or plurality of vesicles isolated or derived from milk is about 200 nm to about 1000 nm, about 200 nm to about 900 nm, about 200 nm to about 800 nm, about 200 nm to about 700 nm, about 200 nm to about 600 nm, about 200 to about 500 nm, about 200 nm to about 400 nm, about 200 nm to about 300, about 300 to about 1000 nm, about 300 to about 900 nm, about 300 to about 800 nm, about 300 to about 700 nm, about 300 to about 600, about 300 to about 500 nm, about 300 to about 400 nm, about 400 to about 1000 nm, about 400 to about 900, about 400 to about 800 nm, about 400 to about 700 nm, about 400 to about 600 nm, about 400 to about 500 nm, about 500 to about 1000 nm, about 500 to about 900 nm, about 500 to about 800 nm, about 500 about 700 nm, about 500 to about 600 nm, about 600 to about 1000 nm, about 600 to about 900 nm, about 600 to about 800 nm, about 600 to about 700 nm, about 700 to about 1000 nm, about 700 to about 900 nm, about 700 to about 800 nm, about 800 to about 1000 nm, about 800 to about 900 nm, about 900 to about 1000 nm in average size.

The size of the milk vesicles disclosed herein is determined by Dynamic Light Scattering (DLS) or nanoparticle tracking analysis (NTA).

B. Source of Milk Vesicles

The milk vehicles described herein can be derived from any form of milk or milk component of any suitable mammal. The term “milk” as used herein refers to the opaque liquid containing proteins, fats, lactose, and vitamins and minerals that is produced by the mammary glands of mature female mammals including, but not limited to, after the mammals have given birth to provide nourishment for their young. In some embodiments, the term “milk” is further inclusive of colostrum, which is the liquid secreted by the mammary glands of mammals shortly after parturition that is rich in antibodies and minerals. The milk vehicles can be from any mammalian species, including but not limited to, primates (e.g., human, ape, monkey, lemur), rodentia (e.g., mouse, rat, etc), carnivora (e.g., cat, dog, etc.), lagomorpha (e.g., rabbit, etc), cetartiodactyla (e.g., pig, cow, deer, sheep, camel, goat, bufflo, yak, etc.), perissodactyla (e.g., horse, donkey, etc.). In certain embodiments, the milk or colostrum, or vesicles derived therefrom, is from human, cow, buffalo, pig, goat, rat, mouse, sheep, camel, donkey, horse, llama, alpaca, vicuña, reindeer, moose, or yak milk or colostrum. In some embodiments, the milk is cow milk.

Milk as used herein encompass milk of any form, including raw milk (whole milk), colostrum, skim milk, pasteurized milk, homogenized milk, acidified milk (milk with casein removed), or milk component, such as whey.

In some embodiments, the vesicles are derived from colostrum, which is the first form of milk produced by the mammary glands of mammals immediately following delivery of the newborn. In some embodiments, the milk is whole milk or raw milk, which is obtained directly from a female mammal with no further processing. In some embodiments, the milk is fat-free milk or skim milk, which typically has milk fat removed substantially. In some embodiments, the milk is reduced fat milk, e.g., milk having 1% or 2% milk fat. In some embodiments, the milk is pasteurized milk, which is typically prepared by heating milk up and then quickly cooling it down to eliminate certain bacteria. In some embodiments, the milk is HTST (High Temperature Short Time) or flash pasteurized. In some embodiments, the milk is UHT or UP (Ultra High Temperature) pasteurized. In some embodiments, the milk is sterilized milk, for example, irradiated milk. In some embodiments, the milk is homogenized milk, which can be prepared by a process in which the fat molecules in milk (e.g., pasteurized milk) have been broken down so that they stay integrated rather than separating as cream. It is a usually a physical process with no additives. In some embodiments, the milk is processed using a combination of one or more of homogenization, pasteurization, sterilization and/or irradiation.

Methods for homogenization, pasteurization, sterilization, and irradiation of milk are known in the art. For example, methods and machinery or mechanisms for homogenizing milk are known. Homogenization is a mechanical process by which fat globules in the milk are broken down such that they are reduced in size and remain suspended uniformly throughout the milk. Homogenization is accomplished by forcing milk at high pressure through small holes. Other methods of homogenization employ the use of extruders, hammermills, or colloid mills to mill (grind) solids. HTST pasteurization requires heating the milk or colostrum to 165° F. for 15 seconds. UHT or UP pasteurization requires heating the milk or colostrum to 280-284° F. for 2-4 seconds. Milk or colostrum can be irradiated using various methods, including gamma radiation, in which gamma rays emitted from radioactive forms of the element cobalt (Cobalt 60) or of the element cesium (Cesium 137) are used; X-ray radiation, in which x-rays are produced by reflecting a high-energy stream of electrons off a target substance (usually one of the heavy metals) into food; and electron beam or e-beam radiation, in which a stream of high-energy electrons are propelled from an electron accelerator into food.
In some embodiments, the milk can be lyophilized Lyophilized milk can be reconstituted using standard procedures as recommended by manufacturer's instruction and/or as known in the art, for example, by mixing distilled water with lyophilized milk at room temperature such that the milk is present at a final concentration of 5% by weight relative to water.

The milk vesicles described herein can be any types of particles found in milk. Examples include, but are not limited to, lactosome, milk fat globules (MFG), milk exosomes, and whey particles. Lactosome are nanometer-sized lipid-protein particles (˜25 nm) that do not contain triacylglycerol. Argov-Argaman et al., J. Agric Food Chem, 2010, 58(21):11234. MFGs are milk particles having a lipid-protein membrane surrounding milk fat; secreted by milk producing cells; a source of multiple bioactive compounds, such as phospholipids, glycolipids, glycoproteins, and carbohydrates. The milk fat globule is surrounded by a phospholipid trilayer containing associated proteins, carbohydrates, and lipids derived primarily from the membrane of the secreting mammary epithelial cell (lactocyte). This trilayer is collectively known as MFGM. While the MFGM only makes up an estimated 2% to 6% of the total milk fat globule, it is an especially rich phospholipid source, accounting for the majority of total milk phospholipids. In contrast, the inner core of the milk fat globule is composed predominantly of triacylglycerols. Lopez et al., (2011), Colloids and Surfaces. B, Biointerfaces. 83 (1): 29-41. Gallier et al., (2010), Journal of Agricultural and Food Chemistry. 58 (7): 4250-4257. Keenan, T. W. (2001), Journal of Mammary Gland Biology and Neoplasia. 6 (3): 365-371. Milk exosomes refer to extracellular vesicles found in milk, which are secreted by multiple cell types into the extracellular space. Typically, milk exosomes may have a size of about 80-160 nm. Samuel et al., 2017, Sci. Rep. 7:5933. Whey particles are found in milk that contain whey protein.

C. Biological Components of Milk Vesicles

The milk vesicles described herein may comprise one or more of the following molecules: lipid, protein, glycoprotein, glycolipid, lipoprotein, phospholipid, phosphoprotein, peptide, glycan, fatty acid, sterol, steroid, and combinations thereof. Typically, the milk vesicles described herein comprise a lipid-based membrane to which one or more proteins are associated. The proteins may be attached to the surface of the lipid membrane or embedded in the lipid membrane. Alternatively or in addition, the proteins may be encapsulated by the lipid membrane. In some instances, the milk vesicles may contain endogenous RNA, such as miRNA.

Lipid Membrane of Milk Vesicles

The milk vesicle may comprise one or more lipids selected from fatty acid, sterol, steroid, cholesterol, and phospholipid. In some embodiments, the lipid membrane of the milk vesicles described herein may comprise ceramides or derivatives thereof, gangliosides, phosphatidylinositols (PI) such as alpha-lysophosphatidylinositol (LPI), phosphatidylserine (PS), cholesterol (CHOL), phosphatidic acids (PA), glycerol or derivatives thereof, such as diacylglycerol (DAG) or phosphatidylglycerol (PG), sphingolipids, or combinations thereof. Ceramides are a family of lipid molecules composed of sphingosine and a fatty acid. Examples include, but are not limited to, ceramide (Cer), lactosylceramide (LacCer), hexosylceramide (HexCer), and globotriaosylceramide (Gb3). Gangliosides are a family of molecules composed of a glycosphigolipid with one or more sialic acids, for example, n-acetylneuraminic acid (NANA). Examples include, but are not limited to, GM1, GM2, GM3, GD1a, GD1b, GD2, GT1b, GT3, and GQ1. Sphingolipids are a class of lipids containing a backbone of sphingoid bases and a set of aliphatic amino alcohols that includes sphingosine. Examples include sphingomyelin (SM).

Alternatively or in addition, the milk vesicles may contain lipids such as phosphatidylcholines (PC), cholesteryl ester (CE), phosphatidylethanolamine (PE), and/or lysophosphatidylethanolamine (LPE).

Proteins, Polypeptides, and Peptides of Milk Vesicles

The milk vesicles described herein may comprise one or more proteins, which may be associated with the lipid membranes also described herein. A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. In some instances, a peptide may contain ten or more amino acids but less than 50. In some instances, a polypeptide or a protein may contain 50 or more amino acids. In other instances, a peptide, polypeptide, or protein may have a mass from about 10 kDa to about 30 kDa, or about 30 kDa to about 150 or to about 300 kDa.

Exemplary proteins may contain only natural amino acids, although non natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

In some embodiments, the milk vesicle comprises one or more polypeptides selected from the following polypeptides: butyrophilin subfamily 1, butyrophilin subfamily 1 member A1, butyrophilin subfamily 1 member A1 isoform X2, butyrophilin subfamily 1 member A1 isoform X3, serum albumin, fatty-acid binding protein, fatty acid binding protein (heart), lactadherin, lactadherin isoform X1, beta-lactoglobin, beta-lactoglobin precursor, lactotransferrin precursor, alpha-S1-casein isoform X4, alpha-S2-casein precursor, casein, kappa-casein precursor, alfa-lactalbumin precursor, platelet glycoprotein 4, xanthine dehydrogenase oxidase, ATP-binding cassette sub-family G, perilipin, perilipin-2 isoform X1, RAB1A (member RAS oncogene family), peptidyl-prolyl cis-trans isomerase A, ras-related protein RAB-18, EpCam, CD81, TSG101, HSP70, polymeric immunoglobulin receptor, lactoferrin, CD63, Tsg101, Alix, CD81, and lactoperoxidase isoform X1. In some embodiments, the milk vesicle comprises butyrophilin In some embodiments, the milk vesicle comprises butyrophilin subfamily 1. In some embodiments, the milk vesicle comprises butyrophilin subfamily 1 member A1. In some embodiments, the milk vesicle comprises lactadherin. In some embodiments, the milk vesicle comprises one or more of the following polypeptides: CD81, CD63, Tsg101, CD9, Alix, EpCAM. In some embodiments, the milk vesicle may comprise a fragment of any of the proteins disclosed herein, for example, the transmembrane fragment. In particular examples, the milk vesicle may comprise BTN1A1, BTN1A2, or a combination thereof. One or more of these polypeptides may enhance the stability, loading of cargo, transport, uptake into cells or tissues, and/or bioavailability of the milk vesicle.

Any of the protein moieties in the milk vesical may be glycosylated, i.e., linked to one or more glycans at one or more glycosylation sites. A glycan is a compound consisting of one or more monosaccharides linked glycosidically, including for example, the carbohydrate portion of a glycoconjugate, such as a glycoprotein, glycolipid, or a proteoglycan. Glycans can be homo- or heteropolymers of monosaccharide residues and can be linear or branched. Glycans can have O-glycosidic linkages (linked to oxygen in a serine or threonine residue of a peptide chain) or N-Linked linkages (linked to nitrogen in the side chain of asparagine in the sequence Asn-X-Ser or Asn-X-Thr, where X is any amino acid except proline). Glycans bind lectins and have many specific biological roles in cell-cell recognition and cell-matrix interactions.

The glycosylated proteins that can be present in the biological membrane of a milk vesicle as described herein can include any appropriate glycan. Examples of glycans include, without limitation, N-glycans (e.g., N-acetyl-glucosamines and N-glycan chains), O-glycans, C-glycans, sialic acid, galactose or mannose residues, and combinations thereof. In some embodiments, the glycan is selected from an alpha-linked mannose, Gal β 1-3 GalNAc 1 Ser/Thr, GalNAc, or sialic acid. In some embodiments, the milk vesicle comprises one or more glycoproteins or glycopolypeptides having a glycan selected from: galactose, mannose, O-glycans, N-acetyl-glucosamines, and/or N-glycan chains or any combination thereof. In some embodiments, the milk vesicle comprises one or more glycoproteins or glycopolypeptides having a glycan selected from: D- or L-glucose, erythrose, fucose, galactose, mannose, lyxose, gulose, xylose, arabinose, ribose, 2′-deoxyribose, glucosamine, lactosamine, polylactosamine, glucuronic acid, sialic acid, sialyl-Lewis X (SLex), N-acetyl-glucosamine, N-acetyl-galactosamine, neuraminic acid, N-glycolylneuraminic acid (Neu5Gc), N-acetylneuraminic acid (Neu5Ac), an N-glycan chain, an O-glycan chain, a Core 1, Core 2, Core 3, or Core 4 structure, or a phosphate- or acetate-modified analog thereof or a combination thereof. In some embodiments, the milk vesicle comprises a glycoprotein having one or more of the following glycans: terminal b-galactose, terminal a-galactose, N-acetyl-D-galactosamine, N-acetyl-D-galactosamine, and N-acetyl-D-glucosamine

In some instances, any of the glycans described herein may exist in free form in the milk vesicles, which are also within the scope of the present disclosure.

In some embodiments, the milk vesicles or a composition comprising such contain proteins having a molecule weight of about 25-30 kDa at a relative abundance of no greater than 40% (e.g., less than about 40%, about 35%, about 30%, about 25%, about 20%, about 15%, about 10%, about 5% or less). In some instances, the proteins having a molecule weight of about 25-30 kDa are caseins. In some examples, the milk vesicles or the composition comprising such may be substantially free of casein, e.g., cannot be detected by a conventional method or only a trace amount can be detected by the conventional method. Alternatively or in addition, the milk vesicles or a composition comprising such contain proteins having a molecule weight of about 10-20 kDa at a relative abundance of no greater than 25% (e.g., less than about 25%, about 20%, about 15%, about 10%, about 5% or less). In some instances, the proteins having a molecule weight of about 10-20 kDa are lactoglobulins. In some examples, the milk vesicles or the composition comprising such may be substantially free of lactoglobulins.

As used herein, the term “casein” refers to a family of related phosphoprotein commonly found in mammalian milk having a molecular weight of about 25-30 kDa. Exemplary species include alpha-S1-casein (αS1), alpha-S2-casein (αS2), β-casein, κ-casein. A casein protein may refer to a specific species as known in the art, for example, those noted above. Alternatively, it may refer to a mixture of at least two different species. In some instances, casein can be the population of all casein proteins found in the milk of a mammal, for example, any of those described herein (e.g., cow, goat, sheep, yak, buffalo, camel, or human).

Lactoglobulin, including α-lactoglobulin and β-lactoglobulin, is a family of whey proteins found in mammalian milk having a molecule weight of about 10-20 kDa. β-lactoglobulin typically has a molecular weight of about 18 kDa and α-lactoglobulin typically has a molecular weight of about 15 kDa. The term “lactoglobulin” may refer to one particular species, e.g., α-lactoglobulin or β-lactoglobulin. Alternatively, it may refer to a mixture of different species, for example, a mixture of α-lactoglobulin and β-lactoglobulin.

Besides the other features disclosed herein (e.g., stability), casein and/or lactoglobuin-depleted milk vesicles or compositions comprising milk vesicles have a higher cargo loading capacity such as oligonucleotide loading capacity as compared with milk vesicles prepared by the conventional ultracentrifugation method.

D. Stability of Milk Vesicles

The milk vesicles described herein are stable under, for example, harsh conditions, e.g., low or high pH, sonication, enzyme digestion, freeze-thaw cycles, temperature treatment, etc. Stable or stability means that the milk vesicles maintain substantially the same intact physical structures and substantially the same functionality as relative to the milk vesicles under normal conditions. For example, a substantial portion of the milk vesicles (e.g., at least 60%, at least 70%, at least 80%, at least 90%, or above) would have no substantial structural changes when they are placed under an acidic condition (e.g., pH≤6.5) for a period of time. Alternatively or in addition, the milk vesicles may be resistant to enzymatic digestion such that a substantial portion of the milk vesicles (e.g., at least 60%, at least 70%, at least 80%, at least 90%, or above) would have no substantial structural changes in the presence of enzymes such as digestive enzymes. Further, the milk vesicles that are stable after multiple rounds of freeze-thaw cycles (e.g., up to 6 cycles) would have a substantial portion (e.g., at least 60%, at least 70%, at least 80%, at least 90%, or above) that has no substantial structural changes and/or functionality changes after the multiple freeze-thaw cycles. Because of, at least in part, the stability of the milk vesicles described herein, such milk vesicles are able to deliver their cargo while withstanding stressed conditions or conditions under which the therapeutic agent would become deactivated, metabolized, or decomposed, e.g., saliva, digestive enzymes, acidic conditions in the stomach, peristaltic motions, and/or exposure to the various digestive enzymes, for example, proteases, peptidases, lipases, amylases, and nucleases that break down ingested components in the gastrointestinal tract.

In some embodiments, the milk vesicle is stable in the gut or gastrointestinal tract of a mammalian species. In some embodiments, the milk vesicle is stable in the esophagus of a mammalian species. In some embodiments, the milk vesicle is stable in the stomach of a mammalian species. In some embodiments, the milk vesicle is stable in the small intestine of a mammalian species. In some embodiments, the milk vesicle is stable in the large intestine of a mammalian species. In some embodiments, the milk vesicle is stable at a pH range of about pH 1.5 to about pH 7.5. In some embodiments, the milk vesicle is stable at a pH range of about pH 2.5 to about pH 7.5. In some embodiments, the milk vesicle is stable at a pH range of about pH 4.0 to about pH 7.5. In some embodiments, the milk vesicle is stable at a pH range of about pH 4.5 to about pH 7.0. In some embodiments, the milk vesicle is stable at a pH range of about pH 1.5 to about pH 3.5. In some embodiments, the milk vesicle is stable at a pH range of about pH 2.5 to about pH 3.5. In some embodiments, the milk vesicle is stable at a pH range of about pH 2.5 to about pH 6.0. In some embodiments, the milk vesicle is stable at a pH range of about pH 4.5 to about pH 6.0. In some embodiments, the milk vesicle is stable at a pH range of about pH 6.0 to about pH 7.5. In some embodiments, the milk vesicle is stable at a pH range of 1.5-7.5. In some embodiments, the milk vesicle is stable at a pH range of 2.5-7.5. In some embodiments, the milk vesicle is stable at a pH range of 4.0-7.5. In some embodiments, the milk vesicle is stable at a pH range of 4.5-7.0. In some embodiments, the milk vesicle is stable at a pH range of 1.5-3.5. In some embodiments, the milk vesicle is stable at a pH range of 2.5-3.5. In some embodiments, the milk vesicle is stable at a pH range of 2.5-pH 6.0. In some embodiments, the milk vesicle is stable at a pH range of 4.5-6.0. In some embodiments, the milk vesicle is stable at a pH range of 6.0-7.5. In some embodiments, the milk vesicle is stable at about pH 1.5, pH 2.0, pH 2.5, pH 3.0, pH 3.5, pH 4.0, pH 4.5, pH 5.0, pH 5.5, pH 6.0, pH 6.5, pH 7.0, or pH 7.5, and increments between about pH of 1.5 and about pH 7.5.

In some embodiments, the milk vesicle is stable in the presence of digestive enzymes, such as, for example, proteases, peptidases, nucleases, pepsin, pepsinogen, lipase, trypsin, chymotrypsin, amylase, bile and pancreatin (digestive enzymes in pancreas). In some embodiments, the milk vesicle is stable in the presence of pepsin or pancreatin. In particular embodiments, the milk vesicles disclosed herein can protect cargo loaded therein (e.g., oligonucleotides) from enzyme digestion (e.g., nuclease digestion).

In some embodiments, the milk vesicles disclosed herein are stable after multiple rounds of freeze-thaw cycles. For example, the milk vesicles are stable after at least two freeze-thaw cycles, e.g., at least 3 cycles, at least 4 cycles, at least 5 cycles, or at least 6 cycles. In some instances, the milk vesicles are stable up to 10 freeze-thaw cycles, e.g., up to 9 cycles, upto to 8 cycles, up to 7 cycles, or up to 6 cycles.

In some embodiments, the milk vesicles disclosed herein are stable after temperature treatment, e.g., incubated at a low temperature (e.g., at 4° C.) for a period (e.g., 1-3 days) or at a high temperature for period, e.g., at 60-80° C. for 30 minutes to 2 hours or at 100-120° C. for 5-20 minutes.

Further, the milk vesicles disclosed herein have colloidal stability. Colloidal stability refers to the long-term integrity of a dispersion and its ability to resist phenomena such as sedimentation or particle aggregation. This is typically defined by the time that dispersed phase particles can remain suspended without producing precipitates.

Alternatively or in addition, the milk vesicles may be stable under physical processes, for example, sonication, centrifugation, and filtration.

E. Modification of Milk Vesicles

In some embodiments, the milk vesicle is a natural (unmodified) milk vesicle. In some embodiments, the milk vesicle is modified to alter one or more lipids, proteins, glycoproteins, glycolipids, lipoproteins, phospholipids, phosphoproteins, peptides, glycans, fatty acids, and/or sterols present in the natural milk vesicle. In some embodiments, the milk vesicle is modified by altering the quantity, concentration, or amount of a biomolecule naturally present, e.g., the addition or complete or partial removal of a biomolecule naturally present (e.g., carbohydrate, such as a glycan; fatty acid, lipid). In some embodiments, the milk vesicle is modified by the addition of a biomolecule not naturally present (e.g., carbohydrate, such as a glycan; fatty acid; lipid).

In some embodiments, the milk vesicle is modified to alter one or more lipids in the milk vesicle. In some embodiments, the lipid component of the milk vesicle is modified or altered, e.g., via the addition of one or more lipids not naturally present in the milk vesicle or by altering the amount (increasing or decreasing) of one or more lipids naturally present in the milk vesicle. In some embodiments, the milk vesicle is modified to increase one or more lipids selected from one or more of the following lipids: LPE, PEO/PEP, Cer, DAG, GM2, PA, Gb3, LacCer, GM1, GM3, HexCer, GD1, PS, Chol, LPI, and SM. The lipid component of the milk vesicle can be altered or modified by known methods, including, for example, fusion with another vesicle having a lipid bilayer, e.g., liposome and/or lipid nanoparticle.

In some embodiments, the milk vesicle comprises one or more glycoproteins. In some embodiments, the milk vesicle comprises a biological membrane, wherein the biological membrane comprises one or more glycoprotein(s). In some embodiments, the biological membrane is modified as compared with the natural biological membrane of the milk vesicle. In some embodiments, the biological membrane is modified such that it has an increased number of one or more of its native glycoprotein(s). In some embodiments, the biological membrane is modified such that it has a decreased number of one or more of its native glycoprotein(s). In some embodiments, the milk vesicle is modified such that it includes one or more glycoprotein(s) that is not naturally present in the natural biological membrane.

In some embodiments, a milk vesicle having a decreased number of one or more of its native glycoprotein(s) is produced using an enzyme selected from a serine protease, cysteine protease or metalloprotease. In some embodiments, the enzyme is selected from trypsin, AspN, GluC, ArgC, chymotrypsin, proteinase K, and Lys-C. In some embodiments, the biological membrane is modified such that one or more of its native glycoprotein(s) is eliminated or not present. In some embodiments, the biological membrane is modified such that one or more of its native glycoprotein(s) is reduced.

In some embodiments, the milk vesicle is modified to alter the amount or content of carbohydrate moieties present on a glycopolypeptide present in or associated with the milk vesicle. In some embodiments, the milk vesicle is modified to increase, decrease, or otherwise alter the glycan content of the milk vesicle, e.g., via the addition of one or more glycans not naturally present in the milk vesicle or by altering the amount (increasing or decreasing) of one or more glycans naturally present in the milk vesicle.

In some embodiments, the biological membrane of the milk vesicle is modified such that one or more of its native glycoprotein(s) is altered. In some embodiments, the one or more native glycoprotein(s) is altered such that the number of glycan residues present on the glycoprotein(s) is increased. In some embodiments, the milk vesicle is produced using glycosylation that adds one or more glycans to the glycoprotein. In some embodiments, the milk vesicle is modified to increase one or more glycoprotein(s) having one or more of the following glycans: terminal b-galactose, terminal α-galactose, N-acetyl-D-galactosamine, N-acetyl-D-galactosamine, and N-acetyl-D-glucosamine.

In some embodiments, the one or more native glycoprotein(s) is altered such that the number of glycan residues present on the glycoprotein(s) is decreased. In some embodiments, the number of glycan residues is decreased by cleavage of one or more glycan residues present on the glycoprotein(s). In some embodiments, the milk vesicle is produced using an enzyme selected from a glycosidase, exoglycosidase, endoglycosidase, glycoamidase, neuraminidase, galactosidase, peptide: N-glycosidase (PNGase), glycohydrolase, and any combination thereof wherein the milk exosome is contacted with the enzyme to remove one or more glycans. In some embodiments, the enzyme is selected from a β-N-acetylglucosaminidase, PNGase F, β (1-4) Galactosidase, O-Glycosidase, N-Glycosidase, N-glycohydrolase, Endo H, Endo D, Endo F₂, EndoF₃, and any combination thereof.

In some embodiments, the number of glycan residues is decreased by cleavage of one or more glycan residues present on the glycoprotein(s). In some embodiments, the milk vesicle is produced using an enzyme selected from a glycosidase, exoglycosidase, endoglycosidase, glycoamidase, neuraminidase, galactosidase, peptide: N-glycosidase (PNGase), glycohydrolase, and any combination thereof wherein the milk exosome is contacted with the enzyme to remove one or more glycans. In some embodiments, the enzyme is selected from a β-N-acetylglucosaminidase, PNGase F, β (1-4) Galactosidase, O-Glycosidase, N-Glycosidase, N-glycohydrolase, Endo H, Endo D, Endo F₂, EndoF₃, and any combination thereof.

In some embodiments, two or more native glycoprotein(s) are altered such that at least one glycoprotein has an increased number of glycan residues and at least one other glycoprotein has a decreased number of glycan residues or is missing its glycan residue(s), wherein the glycoprotein(s) having an increased number of glycan residues is different from the glycoprotein(s) having a decreased number of glycan residues or missing glycan residues. In some embodiments, the one or more native glycoprotein(s) is altered such that it comprises a modified glycan. In some embodiments, the modified glycan comprises at least one carbohydrate moiety that differs from that of the glycan in the native glycoprotein(s). In some embodiments, the modified glycan comprises one or more galactose, mannose, O-glycans, N-acetyl-glucosamines, and/or N-glycan chains or any combination thereof. In some embodiments, the glycan is selected from comprises one or more D- or L-glucose, erythrose, fucose, galactose, mannose, lyxose, gulose, xylose, arabinose, ribose, 2′-deoxyribose, glucosamine, lactosamine, polylactosamine, glucuronic acid, sialic acid, sialyl-Lewis X (SLex), N-acetyl-glucosamine, N-acetyl-galactosamine, neuraminic acid, N-glycolylneuraminic acid (Neu5Gc), N-acetylneuraminic acid (NeuSAc), an N-glycan chain, an O-glycan chain, a Core 1, Core 2, Core 3, or Core 4 structure, or a phosphate- or acetate-modified analog thereof or a combination thereof. In some embodiments, the modified glycan lacks a portion of one or more of its carbohydrate chain(s). In some embodiments, the modified glycan is missing one or more of its carbohydrate chain(s). In some embodiments, the modified glycan comprises one or more altered carbohydrate chain(s). In some embodiments, the one or more native glycoprotein(s) is altered such that at least one glycan present on the glycoprotein(s) is substituted with a glycan that is not naturally present in the native glycoprotein(s). See, also, WO2018170332, the relevant disclosures of which are incorporated by reference for the purpose and subject matter referenced herein.

In some embodiments, altering the number or content of the glycan residues on the milk vesicle affects the colloidal stability of the milk vesicle. In some embodiments, altering the number or content of the glycan residues on the milk vesicle modulates the interaction between milk vesicles and GI cells, e.g., enhances the uptake of milk vesicles in GI cells.

Modifications to the milk vesicles as described herein can be made via conventional methods. For example, milk vesicles isolated from a natural source may be subject to extrusion (e.g., once or multiple times) through a filter having a suitable size, e.g., 50 nM, 75 nM, or 100 nM, to change size distribution. In another example, milk vesicles isolated from one or more natural sources may be subject to homogenization (e.g., under high pressure in some instances) to cause fusion of particles. Alternatively, extrusion or homogenization may be performed to milk vesicles isolated from a natural source in the presence of other natural or artificial lipid membrane vesicles or protein micelles or aggregates to produce fused particles. Such fusion may lead to change of protein and/or lipid content of the resultant particles, for example, incorporating non-naturally occurring lipids, which may present in the artificial lipid membrane particles. In another example, additional lipids may be incorporated into milk vesicles isolated from a natural source via saturation of the milk vesicles with specific lipids of interest or incubating the milk vesicles with lipid films, which may contain lipids of interest (e.g., cholesterol, phospholipids, ceramides, and/or sphingomyelins.).

In addition, milk vesicles isolated from a natural source may be modified by removing certain lipid contents. For example, methyl-beta-cyclodextrin can be used to extract cholesterol from milk vesicles.

In addition, milk vesicles may be modified by conjugating suitable moieties, such as proteins, polypeptides, peptides, glycans, etc. onto surface proteins of the milk vesicles, via conventional methods.

### II. Cargo-Loaded Milk Vesicles

Any of the milk vesicles described herein can be used as vehicles for carrying biological molecules (cargo) to facilitate delivery of the biological molecules to a subject. In addition to other superior features of milk vesicles disclosed herein, the milk vesicles can protect the cargo loaded therein from degradation, for example, from digestion by enzymes. Thus, also provided herein are cargo-loaded milk vesicles, which can be used to deliver (e.g., orally) the loaded cargo to a subject for diagnostic and/or therapeutic purposes. In some instances, the cargo is a therapeutic agent.

In some embodiments, the present disclosure provides a cargo-loaded vesicle or a therapeutic-loaded vesicle. The term “cargo-loaded vesicle,” “therapeutic-loaded vesicle” or “therapeutic agent-loaded vesicle” is meant to be inclusive of the loading of one or more cargos, including therapeutic agents and diagnostic agents. As used herein, the term “loaded” or “loading” as used in reference to a “cargo-loaded vesicle,” “therapeutic-loaded vesicle” or “therapeutic agent-loaded vesicle” refers to a vesicle having one or more cargos (which can be biological molecules such as therapeutic agents or diagnostic agents) that are either (1) encapsulated inside the vesicle; (2) associated with or partially embedded within the lipid membrane of the vesicle (i.e. partly protruding inside the interior of the vesicle); (3) associated with or bound to the outer portion of the lipid membrane and associated components (i.e., partly protruding or fully outside the vesicle); or (4) entirely disposed within the lipid membrane of the vesicle (i.e., entirely contained within the lipid membrane).

The term “cargo-loading” refers to the process of loading, adding, or including exogenous cargo or therapeutic to the milk vesicle such that any one or more of the above (1)-(4) resultant cargoloaded or therapeutic-loaded vesicles is accomplished. Thus, in some embodiments, the therapeutic agent is encapsulated inside the vesicle. In some embodiments, the therapeutic agent is associated with or partially embedded within the lipid membrane of the vesicle (i.e. partly protruding inside the interior of the vesicle). In some embodiments, the therapeutic agent is associated with or bound to the outer portion of the lipid membrane (i.e., partly protruding outside the vesicle). In some embodiments, the therapeutic agent is entirely disposed within the lipid membrane of the vesicle (i.e., entirely contained within the lipid membrane). As used herein, the term “cargo” is meant to include any biomolecule or agent that can be loaded into or by a milk vesicle, including, for example, a biologic, small molecule, therapeutic agent, and/or diagnostic agent.

In some embodiments, one or more cargos, e.g., therapeutic agent, are present on the interior or internal surface of the milk vesicle. In some embodiments, the one or more agents, e.g., therapeutic agent, present on the interior or internal surface of the milk vesicle are associated with the milk vesicle, e.g., via chemical interaction, electromagnetic interaction, hydrophobic interaction, electrostatic interaction, van der Waals interaction, linkage, bond (hydrogen bond, ionic bond, covalent bond, etc.). In some embodiments, the one or more agents, e.g., therapeutic agent, present on the interior or internal surface of the milk vesicle are not associated with the milk vesicle, e.g., the agent is unattached to the vesicle. In some embodiments, the milk vesicle has a cavity and/or forms a sac. In some embodiments, the milk vesicle can encapsulate one or more agents, e.g., therapeutic agents.

In some embodiments, the one or more cargos, e.g., therapeutic agent, are present on the exterior or external surface of the vesicle. In some embodiments, the one or more agents, e.g., therapeutic agent, present on the exterior or external surface of the vesicle are associated with the milk vesicle, e.g., via chemical interaction, electromagnetic interaction, hydrophobic interaction, electrostatic interaction, van der Waals interaction, linkage, bond (hydrogen bond, ionic bond, covalent bond, etc.). In some embodiments, the therapeutic agent is conjugated to a hydrophobic group such as a sterol, steroid, or lipid. In some embodiments, the hydrophobic group facilitates loading of the therapeutic agent into the milk vesicle and/or delivery of the therapeutic agent to a target tissue or organ.

In some embodiments, the milk vesicle is loaded with a single cargo, for example, a single therapeutic agent. In some embodiments, the milk vesicle is loaded with two (or more) different therapeutic agents. In some embodiments, the milk vesicle is loaded with two or more molecules or copies of a single therapeutic agent or two (or more) different therapeutic agents. In some embodiments, the milk vesicle is loaded with three or more molecules or copies of a single therapeutic agent or two (or more) different therapeutic agents. In some embodiments, the milk vesicle is loaded with 2-5 molecules or copies of a single therapeutic agent or two (or more) different therapeutic agents. In some embodiments, the milk vesicle or pharmaceutical composition thereof is loaded with 1-4,000, 10-4,000, 50-3,500, 100-3,000, 200-2,500, 300-1,500, 500-1,200, 750-1,000, 1-2,000, 1-1,000, 1-500, 10-400, 50-300, 1-250, 1-100, 2-50, 2-25, 2-15, 2-10, 3-50, 3-25, 3-25, 3-10, 4-50, 4-25, 4-15, 4-10, 5-50, 5-25, 5-15, or 5-10 molecules or copies of a single therapeutic agent or two (or more) different therapeutic agents, or any increment therein.

A. Cargo

The cargo (biological molecule) in the cargo-loaded milk vesicles described herein can be of any type. Examples include, but are not limited to, proteins, nucleic acids, lipids, carbohydrates, and small molecules. The cargo may be a biological molecule that is not naturally-occurring in a milk vesicle, e.g., has been modified as described herein.

In some embodiments, the biological molecule is a biologic agent. As used herein, the term “biologic” is used interchangeably with the term “biologic therapeutic agent”. One of ordinary skill in the art will recognize that such biologics include those described herein. In some examples, the biologic therapeutic agent can be an allergen, adjuvant, antigen, or immunogen. Examples include autoimmune antigen and food allergen. In other examples, the biologic therapeutic agent can be an antibody, hormone, factor, cofactor, metabolic enzyme, immunoregulatory enzyme, interferon, interleukin, gastrointestinal enzyme, an enzyme or factor implicated in hemostasis, growth regulatory enzyme, vaccine, antithrombotic, antithrombolytic, toxin, or an antitoxin.

(i) Nucleic Acids

In other embodiments, the biological molecule is a nucleic acid, for example, an oligonucleotide therapeutic agent, such as a single-stranded or double-stranded oligonucleotide therapeutic agent. In some examples, the oligonucleotide therapeutic agent can be a single-stranded or double-stranded DNA, iRNA, siRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof.

In some embodiments, the nucleic acid is a ncRNA of about 30 to about 200 nucleotides (nt) in length or a long non-coding RNA (lncRNA) of about 200 to about 800 nt in length. In some embodiments, the lncRNA is a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In some embodiments, the ncRNA is selected from a piwi-interacting RNA (piRNA), primary miRNA (pri-miRNA), or premature miRNA (pre-miRNA).

In some examples, the present disclosure provides the following lipid-modified double-stranded RNA that may be loaded in and delivered by the milk vesicles described herein. In some embodiments, the RNA is one of those described in CA 2581651 or U.S. Pat. No. 8,138,161, each of which is hereby incorporated by reference in its entirety.

ncRNA and lncRNA

The broad application of next-generation sequencing technologies in conjunction with improved bioinformatics has helped to illuminate the complexity of the transcriptome, both in terms of quantity and variety. In humans, 70-90% of the genome is transcribed, but only ˜2% actually codes for proteins. Hence, the body produces a huge class of non-translated transcripts, called long non-coding RNAs (lncRNAs), which have received much attention in the past decade. Recent studies have illuminated the fact that lncRNAs are involved in a plethora of cellular signaling pathways and actively regulate gene expression via a broad selection of molecular mechanisms.

Human and other mammalian genomes pervasively transcribe tens of thousands of long non-coding RNAs (lncRNAs). The latest edition of data produced by the public research consortium GenCode (version #27) catalogs just under 16,000 lncRNAs in the human genome, producing nearly 28,000 transcripts; when other databases are included, more than 40,000 lncRNAs are known.

These mRNA-like transcripts have been found to play a controlling role at nearly all levels of gene regulation, and in biological processes like embryonic development. A growing body of evidence also suggests that aberrantly expressed lncRNAs play important roles in normal physiological processes as well as multiple disease states, including cancer. lncRNAs are a group that is commonly defined as transcripts of more than 200 nucleotides (e.g., about 200 to about 1200 nt, about 2500 nt, or more) that lack an extended open reading frame (ORF). The term “non-coding RNA” (ncRNA) includes lncRNA as well as shorter transcripts of, e.g., less than about 200 nt, such as about 30 to 200 nt. Several lncRNAs, e.g. gadd74 and lncRNA-RoR5, modulate cell cycle regulators such as cyclins, cyclin-dependent kinases (CDKs), CDK inhibitors and p53 and thus provide an additional layer of flexibility and robustness to cell cycle progression. In addition, some lncRNAs are linked to mitotic processes such as centromeric satellite RNA, which is essential for kinetochore formation and thus crucial for chromosome segregation during mitosis in humans and flies. Another nuclear lncRNA, MA-linc1, regulates M phase exit by functioning in cis to repress the expression of its neighboring gene Purα, a regulator of cell proliferation. Since deregulation of the cell cycle is closely associated with cancer development and growth, cell cycle regulatory lncRNAs may have oncogenic properties.

Thus, in some embodiments, delivery of a ncRNA, such as to a specific tissue or organ of interest, corrects aberrant RNA expression levels or modulates levels of disease-causing lncRNA. Accordingly, in some embodiments, the present invention provides a therapeutic-loaded milk vesicle, wherein the therapeutic is a non-coding RNA (ncRNA). In some embodiments, the ncRNA is a long non-coding RNA (lncRNA) of about 200 nucleotides (nt) in length or greater. In some embodiments, the therapeutic is a ncRNA of about 25 nt or about 30 nt to about 200 nt in length. In some embodiments, the lncRNA is about 200 nt to about 1,200 nt in length. In some embodiments, the lncRNA is about 200 nt to about 1,100, about 1,000, about 900, about 800, about 700, about 600, about 500, about 400, or about 300 nt in length.

Micro RNA (miRNA)

In some embodiments, the therapeutic is a miRNA. As would be recognized by those skilled in the art, miRNAs are small non-coding RNAs that are about 17 to about 25 nucleotide bases (nt) in length in their biologically active form. In some embodiments, the miRNA is about 17 to about 25, about 17 to about 24, about 17 to about 23, about 17 to about 22, about 17 to about 21, about 17 to about 20, about 17 to about 19, about 18 to about 25, about 18 to about 24, about 18 to about 23, about 18 to about 22, about 18 to about 21, about 18 to about 20, about 19 to about 25, about 19 to about 24, about 19 to about 23, about 19 to about 22, about 19 to about 21, about 20 to about 25, about 20 to about 24, about 20 to about 23, about 20 to about 22, about 21 to about 25, about 21 to about 24, about 21 to about 23, about 22 to about 25, about 22 to about 24, or about 22 nt in length. miRNAs regulate gene expression post-transcriptionally by decreasing target mRNA translation. It is thought that miRNAs function as negative regulators.

There are generally three forms of miRNAs: primary miRNAs (pri-miRNAs), premature miRNAs (pre-miRNAs), and mature miRNAs. Primary miRNAs are expressed as stem-loop structured transcripts of about a few hundred bases to over 1 kb. The pri-miRNA transcripts are cleaved in the nucleus by Drosha, an RNase II endonuclease that cleaves both strands of the stem near the base of the stem loop. Drosha cleaves the RNA duplex with staggered cuts, leaving a 5′ phosphate and 2 nt overhang at the 3′ end. The cleaved product, the premature miRNA (pre-miRNA) is about 60 to about 110 nt long with a hairpin structure formed in a fold-back manner Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nt in length. In some embodiments, the miRNAs encapsulated by the microvesicles of the presently-disclosed subject matter are selected from miR-155, which is known to act as regulator of T- and B-cell maturation and the innate immune response, or miR-223, which is known as a regulator of neutrophil proliferation and activation. Other non-natural miRNAs such as iRNAs (e.g. siRNA) or natural or non-natural oligonucleotides may be present in the milk-derived vesicles and represent an encapsulated therapeutic agent, as the term is used herein.

Short Interfering RNA (siRNA)

In some embodiments, the therapeutic is a siRNA. Small interfering RNA (siRNA), sometimes known as short interfering RNA or silencing RNA, is a class of double-stranded RNA molecules, 20-25 base pairs in length (of similar length to miRNA). siRNAs generally exert their biological effects through the RNA interference (RNAi) pathway. siRNAs generally have 2 nucleotide overhangs that are produced through the enzymatic cleavage of longer precursor RNAs by the ribonuclease Dicer. siRNAs can limit the expression of specific genes by targeting their RNA for destruction through the RNA interference (RNAi) pathway. It interferes with the expression of specific genes with complementary nucleotide sequences by degrading mRNA after transcription, preventing translation. siRNA can also act in RNAi-related pathways as an antiviral mechanism or play a role in the shaping of the chromatin structure of a genome.

In some examples, the RNA is an siRNA molecule comprising a modified ribonucleotide, wherein said siRNA (a) comprises a two base deoxynucleotide “TT” sequence at its 3′ end, (b) is resistant to RNase, and (c) is capable of inhibiting viral replication. In some examples, the siRNA molecule is 2′ modified. In some embodiments, the 2′ modification is selected from the group consisting of fluoro-, methyl-, methoxyethyl- and propyl-modification. In some embodiments, the fluoro-modification is a 2′-fluoro-modification or a 2′,2′-fluoro-modification.

In some embodiments, at least one pyrimidine of the siRNA is modified, and said pyrimidine is cytosine, a derivative of cytosine, uracil, or a derivative of uracil. In some embodiments, all of the pyrimidines in the siRNA are modified. In some embodiments, both strands of the siRNA contain at least one modified nucleotide. In some embodiments, the siRNA consists of about 10 to about 30 ribonucleotides. In some embodiments, the siRNA molecule is consists of about 19 to about 23 ribonucleotides.

In some embodiments, the siRNA molecule comprises a nucleotide sequence at least 80% identical to the nucleotide sequence of siRNA5, siRNAC1, siRNAC2, siRNA5B1, siRNA5B2 or siRNA5B4. In some embodiments, the siRNA molecule is linked to at least one receptor-binding ligand. In some embodiments, the receptor-binding ligand is attached to a 5′-end or 3′-end of the siRNA molecule. In some embodiments, the receptor binding ligand is attached to multiple ends of said siRNA molecule. In some embodiments, the receptor-binding ligand is selected from the group consisting of a cholesterol, an HBV surface antigen, and low-density lipoprotein. In some embodiments, the receptor-binding ligand is cholesterol.

In some embodiments, the siRNA molecule comprises a modification at the 2′ position of at least one ribonucleotide, which modification at the 2′ position of at least one ribonucleotide renders said siRNA resistant to degradation. In some embodiments, the modification at the 2′ position of at least one ribonucleotide is a 2′-fluoro-modification or a 2′,2′-fluoro-modification.

In an embodiment, the present disclosure provides a double-stranded (dsRNA) molecule that mediates RNA interference in target cells wherein backbone sugars of one or more of the pyrimidines in the dsRNA are modified to include a 2′-fluorine, a 2′-O-methyl, a 2′-MOE, a phosphorothioate bond (e.g., including stereoisomers of those and other modifications of phosphodiether bonds, bridged nucleotides, e.g., locked nucleotides), or a combination thereof. In some instances, the modification may include inverted bases and/or abasic nucleotides. Alternatively or in addition, the modifications may include peptide nucleic acids (PNAs), such as gamma-PNAs and/or PNA-oligopeptide hybrids. Any of the modifications described herein may apply to other types of nucleic acid moelcules as also disclosed herein where applicable.

Any of the nucleic acid-based cargo molecules disclosed herein may comprise one or more modifications at any position applicable. For example, non-limiting examples of modifications can comprise one or more nucleotides modified at the 2′-position of the sugar, e.g., 2′-Oalkyl, 2′-O-alkyl-O-alkyl, or 2′-fluoro-modified nucleotide. In some embodiments, modifications to an RNA molecule may include 2′-fluoro, 2′-amino or 2′-O-methyl modifications on he ribose of one or more pyrimidines, abasic residues, desoxy nucleotides, or an inverted base at the 3′ end of the RNA molecule. Alternatively or in addition, the nucleic acid-based cargo molecule may include one or more modifications in the bockbones such that the modified nucleic acid molecule may be more resistant to nuclease digestion relative to the non-modified counterpart. Such backbone modifications include, but are not limited to, phosphorothioates, phosphorothyos, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, ˜N(CH₃)—O—CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones (wherein the native phosphodiester backbone is represented as O—P—O—CH); amide backbones (De Mesmaeker et al., Ace. Chem. Res. 28:366-374; 1995); morpholino backbone structures (U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone; see, e.g., Nielsen et al., Science 254:1497; 1991). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′-alkylene phosphonates and chiral phosphonates, phosphinates, phosphoramidates comprising 3′-amino phosphoramidate and aminoaklylphosphoramidates, thionophosphoramidates, thionoalkylphosphonates thionoalkylphosphotriesters, and boranophosphates having normal 3′-5′ linkages, 2′-5′ linaged analogs of these, and those having inverted polarity wherein the adjacent pairs of nucleotide units are linked 3′-5′ to 5′-3′ or 2′-5′ to 5′-2′. See, e.g., WO2017/077386, the relevant disclosures of which are incorporated by reference for the purpose and/or subject matter references herein.

In an embodiment, the nucleic acid molecule in any of the milk vesicles described herein is a small interfering RNA (siRNA) that mediates RNA interference in target cells wherein backbone sugars of one or more of the pyrimidines in the siRNA are modified to include a 2′-Fluorine. In an embodiment, all of the backbone sugars of pyrimidines in the dsRNA or siRNA molecules of the first and second embodiments are modified to include a 2′-Fluorine. In an embodiment, the 2′-Fluorine dsRNA or siRNA of the third embodiment is further modified to include a two base deoxynucleotide “TT” sequence at the 3′ end of the dsRNA or siRNA.

In some embodiments, the siRNA molecule is about 10 to about 30 nucleotides long, and mediates RNA interference in target cells. In some embodiments, the siRNA molecules are chemically modified to confer increased stability against nuclease degradation, but retain the ability to bind to target nucleic acids.

The nucleic acid molecules loaded in the milk vesicle also may not be naturally-occurring in the milk from which the milk vesicle is derived. Additional examples include mRNA, antisense RNA, pretranscript, pre-miRNA, pre-mRNA, competing endogenous RNA (ceRNA), small nuclear RNA (snRNA), small nucleolar RNA (snoRNA), pseudo-gene, rRNA, tRNA or other nucleic acids and analogs thereof described herein.

The nucleic acid molecules described herein may target RNAs encoding the following polypeptides: vascular endothelial growth factor (VEGF); Apolipoprotein B (ApoB); luciferase (luc); Androgen Receptor (AR); coagulation factor VII (FVII); hypoxia-inducible factor 1, alpha subunit (Hif-1α); placenta growth factor (PLGF); Lamin A/C; and green fluorescent protein (GFP). Exemplary single stranded oligonucleotide agents are shown in Table 1 below. Additional suitable miRNA targets are described, e.g., in John et al., PLoS Biology 2:1862-1879, 2004 (correction in PLoS 3:1328, 2005), and The microRNA Registry (Griffiths-Jones S., NAR 32:D109-D111, 2004).

Messenger RNAs (mRNAs)

In some embodiments, the therapeutic cargo is an mRNA molecule, which may be a naturally-occurring mRNA or a modified mRNA molecule. In some examples, the mRNA may be modified by introduction of non-naturally occurring nucleosides and/or nucleotides. Any modified nucleosides and/or nucleotides may be used for making the modified mRNA as disclosed herein. Examples include those described in US20160256573, the relevant disclosures are incorporated by reference for the purpose and subject matter referenced herein. In other examples, the mRNA molecule may be modified to have reduced uracil content. See, e.g., US20160237134, the relevant disclosures are incorporated by reference for the purpose and subject matter referenced herein.

(ii) Allergen, Adjuvant, Antigen, or Immunogen

In some embodiments, the therapeutic agent is an allergen, adjuvant, antigen, or immunogen. In some embodiments, the allergen, antigen, or immunogen elicits a desired immune response to increase allergen tolerance or reduce the likelihood of an allergic or immune response such as anaphylaxis, bronchial inflammation, airway constriction, or asthma. In some embodiments, the allergen, antigen, or immunogen elicits a desired immune response to increase viral or pathogenic resistance or elicit an anticancer immune response. In some embodiments, the allergen or antigen elicits a desired immune response to treat an allergic or autoimmune disease. In some embodiments, the therapeutic agent increases immunological tolerance to treat an autoimmune disease or decreases an autoimmune response to treat an autoimmune disease.

As used herein, the term “adjuvant” refers to any substance which enhances an immune response (e.g. in the vaccine, autoimmune, or cancer context) by a mechanism such as: recruiting of professional antigen-presenting cells (APCs) to the site of antigen exposure; increasing the delivery of antigens by delayed/slow release (depot generation); immunomodulation by cytokine production (selection of Th1 or Th2 response); inducing T-cell response (prolonged exposure of peptide-MHC complexes (signal 1) and stimulation of expression of T-cell-activating co-stimulators (signal 2) on an APC surface) and targeting (e.g., carbohydrate adjuvants which target lectin receptors on APCs), and the like.

TABLE 1
Exemplary Oligonucleotide Agents
Exemplaxy oligonucleotide agents

| AL-SQ-NO: | Sequence (5′-3′ unless otherwise indicated) | Target |
|---|---|---|
| 3186 | GCACAUAGGAGAGAUGAGCUUs-Chol | VEGF |
| 3191 | Naproxen-sGUCAUCACACUGAAUACCAAUs-Chol | ApoB |
| 3209 | CAUCACACUGAAUACCAAUdTdTs-Chol | Luc |
| 3230 | oUsoCsoAoCoGoCoGoAoGoCoCoGoAoAoCoGoAoAoCsoAsoAsoAs-Chol | Mir-375 |
| 3234 | oCoUGGGAAAGoUoCAAGoCoCoCAoUdTsdT-Chol | AR |
| 3235 | oCoUGoUGoCAAGoUGoCoCoCAAGAoUdTsdT-Chol | AR |
| 3253 | GGAfUfCAfUfCfUfCAAGfUfCfUfUAfCdTedT-Chol | FVII |
| 3256 | ACUGCAGGGUGAAGAAUUAdTsdTs-Chol | Hif-1α |
| 3257 | GCACAUAGGAGAGAUGAGCUsUs-Chol | VEGF |
| 3258 | GAACUGUGUGUGAGAGGUCCsUs-Chol | Luc |
| 3264 | CCAGGUUUUUUUCUUACUUTsTs-Chol | VEGF |
| 3265 | UUCCUCAAAUCAAUUACCATsTs-Chol | VEGF |
| 3266 | GGAAGGCUCCCUUGAUGGAdTsdTs-Chol | VEGF |
| 3268 | GACACAGUGUGUUUGAUUUdTsdTs-Chol | Hif-1α |
| 3269 | UGCCAAGCCAGAUUCUCUUdTsdTs-Chol | PLGF |
| 3271 | CUCAGGAAUUCAGUGCCUUdTsdTs-Chol | PLGF |
| 3275 | CUGGACUUCCAGAAGAACAdTdT-Chol | Lamin A/C |
| 3150 | Chol-sGUCAUCACACUGAAUACCAAsU | ApoB |
| 5225 | GUCAUCACACUGAAUACCAAUs-Chol | ApoB |
| 4967 | GcACcAUCUUCUUcAAGGACGs-Chol | GFP |
| 5225 | GUCAUCACACUGAAUACCAAUs-Chol | ApoB |
| 5221 | AGGUGUAUGGCUUCAACCCUGs-Chol | ApoB |

In some embodiments, the allergen is selected from a food, animal (e.g. pet such as dog, cat, or rabbit), or environmental allergen (such as dust, pollen, or mildew). In some embodiments, the allergen is selected from abalone, perlemoen, acerola, Alaska pollock, almond, aniseed, apple, apricot, avocado, banana, barley, bell pepper, brazil nut, buckwheat, cabbage, chamomile, carp, carrot, casein, cashew, castor bean, celery, celeriac, cherry, chestnut, chickpea, garbanzo, bengal gram, cocoa, coconut, cod, cotton seed, courgetti, zucchini, crab, date, egg (e.g. hen's egg), fig, fish, flax seed, linseed, frog, garden plum, garlic, gluten, grape, hazelnut, kiwi fruit (chinese gooseberry), legumes, lentil, lettuce, lobster, lupin or lupine, lychee, mackerel, maize (corn), mango, melon, milk (e.g. cow), mollusks, mustard, oat, oyster, peach, peanut (or other ground nuts or monkey nuts), pear, pecan, persimmon, pistachio, pine nuts, pineapple, pomegranate, poppy seed, potato, pumpkin, rice, rye, salmon, sesame, shellfish (e.g. crustaceans, black tiger shrimp, brown shrimp, greasyback shrimp, Indian prawn, neptune rose shrimp, white shrimp), snail, soy, soybean (soya), squid, strawberry, sulfur dioxide (sulfites), sunflower seed, tomato, tree nuts, tuna, turnip, walnut, or wheat (e.g. breadmaking wheat, pasta wheat, kamut, spelt).

In some embodiments, the allergen is selected from an allergenic protein, peptide, oligo- or polysaccharide, toxin, venom, nucleic acid, or other allergen, such as those listed at www.allergenonline.org/databasebrowse.shtml. In some embodiments, the allergen is selected from an airborne fungus, mite or insect allergen, plant allergen, venom or salivary allergen, animal allergen, contact allergen, parasitic allergen, or bacterial airway allergen.

In some embodiments, the therapeutic agent is an autoimmune antigen. In some embodiments, the autoimmune antigen is selected from an antigen against a disease, disorder, or condition listed in Table 2, below. In some embodiments, the antigen is selected from those listed in Table 2, below.

TABLE 2
Exemplary Autoimmune Diseases and Involved Antigens

| AAA Disease Name (101) | Antigen |
|---|---|
| Achlorhydria | against parietal cells which normally produce gastric acid |
| Acute disseminated encephalomyelitis | MOG |
| Addison's Disease | antibodies against 21-hydroxylase enzyme |
| Alopecia areata | antibodies against hair follicles |
| Anemia, Pernicious | antibodies to parietal cells and intrinsic factor |
| Ankylosing spondylitis | Anti-neutrophil cytoplasmic antibodies (ANCAs) |
| Anti - Glomerular Basement Membrane Disease | |
| Anti-GBM/Anti-TBM nephritis | |
| Anti-NMDA receptor encephalitis | N-methyl-D-aspartate receptor (NMDA) |
| Antiphospholipid syndrome (APS) | Antiphospholipid antibodies |
| Aplastic Anemia | |
| Autoimmune Atrophic Gastritis | |
| Autoimmune Hearing Loss | |
| Autoimmune hemolytic anemia | |
| Autoimmune Hepatitis | Antinuclear, anti-mitochondrial and anti-smooth muscle |
| | antibodies, Liver kidney microsomal type 1 antibody, Anti- |
| | smooth muscle antibody |
| Autoimmune hypoparathyroidism | |
| Autoimmune hypophysitis | |
| Autoimmune inner ear disease (AIED) | |
| Autoimmune Lymphoproliferative | |
| Autoimmune Myocarditis | |
| Autoimmune oophoritis | |
| Autoimmune orchitis | spermatozoa normally sequestered in the testis (occurs after |
| | vasectomy) |
| Autoimmune Polyendocrinopathy - Candidiasis - | NA |
| Ectodermal - Dystrophy | |
| Autoimmune Syndrome Type II, Polyglandular | |
| Axonal & neuronal neuropathy (AMAN) | Anti-ganglioside antibodies GD3 |
| Behcet Syndrome | Anti-p62 antibodies, Anti-sp100 antibodies, Anti-glycoprotein- |
| | 210 antibodies |
| Biliary Cirrhosis | Anti-mitochondrial antibody |
| Bullous pemphigoid | |
| Castleman disease (CD) | |
| Celiac disease | Synapsin 1, transglutaminase, gluten |
| Chagas disease | |
| Cholangitis, Sclerosing | |
| Chronic inflammatory demyelinating | |
| polyneuropathy (CIDP) | |
| Chronic lymphocytic thyroiditis | |
| Chronic recurrent multifocal osteomyelitis | |
| (CRMO) | |
| Churg-Strauss syndrome | |
| Cicatricial pemphigoid/benign mucosal | |
| pemphigoid | |
| Cogan's syndrome | |
| Cold agglutinin disease | |
| Colitis, Ulcerative | |
| Congenital heart block | |
| Coxsackie myocarditis | |
| CREST syndrome | Anti-centromere antibodies |
| Crohn's disease | |
| Cryoglobulinemia | |
| Cushing Syndrome | |
| Dermatitis herpetiformis | |
| Dermatomyositis | |
| Devic's disease (neuromyelitis optica) | |
| Diabetes Mellitus, Insulin - Dependent | intracellular islet cell antigens such as glutamic acid |
| | decarboxylase |
| Diabetes, Type 1 | islet cell autoantibodies, insulin autoantibodies, autoantibodies |
| | targeting the 65-kDa isoform of glutamic acid |
| | decarboxylase(GAD), autoantibodies targeting the phosphatase- |
| | related IA-2 molecule, and zinc transporter autoantibodies |
| | (ZnT8) |
| Diffuse Cerebral Sclerosis of Schilder | |
| Discoid lupus | |
| Dressler's syndrome | |
| Encephalomyelitis, Autoimmune, Experimental | |
| Endometriosis | |
| Eosinophilic esophagitis (EoE) | |
| Eosinophilic fasciitis | |
| Epidermolysis Bullosa Acquisita | |
| Erythema nodosum | |
| Erythematosis | |
| Essential mixed cryoglobulinemia | |
| Evans syndrome | |
| Felty's Syndrome | |
| Fibromyalgia | |
| Fibrosing alveolitis | |
| Giant cell arteritis (temporal arteritis) | |
| Giant cell myocarditis | |
| Glomerulonephritis, IGA | renal autoantigen |
| Glomerulonephritis, Membranous | |
| Goodpasture Syndrome | collagen in basement membrane of kidneys and lungs |
| Granulomatosis with polyangiitis | Anti-neutrophil cytoplasmic antibody (C-ANCA) |
| Graves' Disease | antibodies against the TSH receptor, thyroid-stimulating |
| | immunoglobulin (TSI), thyroglobulin or the thyroid hormones |
| | T3 and T4 |
| Guillain - Barre Syndrome | myelin protein |
| HAM/tropical spastic paraparesis | hnRNP A1 |
| Hamman-Rich syndrome | |
| Hashimoto's Thyroidosis | thyroid antigens: (a) Thyroglobulin, (b) Thyroid peroxidase, |
| | (c) TSH receptor, and (d) Iodine transporter |
| Hemolytic anemia | |
| Henoch-Schonlein purpura (HSP) | |
| Hepatitis, Chronic Active | |
| Herpes gestationis or pemphigoid gestationis | |
| (PG) | |
| Hypogammalglobulinemia | |
| Idiopathic thrombocytopenia | |
| IgA Nephropathy | |
| IgG4-related sclerosing disease | |
| Inclusion body myositis (IBM) | |
| Inflammatory Bowel Diseases | |
| Interstitial cystitis (IC) | |
| Juvenile myositis (JM) | |
| Kawasaki disease | |
| Lambert - Eaton Myasthenic Syndrome | voltage-gated calcium channel (P/Q-type) |
| Lens-induced uveitis | |
| Leukocytoclastic vasculitis | |
| Lichen planus | |
| Lichen sclerosus | |
| Lichen Sclerosus et Atrophicus | |
| Ligneous conjunctivitis | |
| Limbic encephalitis | AMPAR (GluR1, GluR2), Anti-Hu (ANNA-1), Lgi1, NMDAR, |
| | NR1/NR2B, voltage-gated potassium channel (VGKC) |
| Linear IgA disease (LAD) | |
| Lupus Erythematosus, Discoid | |
| Lupus Erythematosus, Systemic | double stranded DNA and Smith (Sm) antigen, Anti-histone |
| | antibodies, Anti-SSA/Ro autoantibodies, anti-thrombin |
| | antibodies, NR2A/NR2B, Neuronal surface P antigen |
| Lupus Hepatitis | |
| Lyme disease chronic | |
| Lymphopenia | |
| Meniere's disease | |
| Microscopic polyangiitis (MPA) | Anti-neutrophil cytoplasmic antibody (P-ANCA) |
| Mixed connective tissue disease (MCTD) | anti-Ribonucleoprotein antibodies |
| Mooren's ulcer | |
| Mucha-Habermann disease | |
| Mucocutaneous Lymph Node Syndrome | |
| Multifocal motor neuropathy with conduction | Anti-ganglioside antibodies to GM1 |
| block (MMN) | |
| Multiple Sclerosis | |
| Myasthenia Gravis | nicotinic acetylcholine receptor of neuromuscular junction, |
| | Muscle-specific kinase (MUSK) |
| Myelitis, Transverse | |
| Myocarditis | |
| Myositis | |
| Narcolepsy | |
| Neuritis, Autoimmune, Experimental | |
| Neuromyelitis optica | AQP4, Collapsin response mediator protein 5 |
| Neutropenia | |
| Ocular cicatricial pemphigoid | |
| Oculovestibuloauditory syndrome | |
| Ophthalmia, Sympathetic | |
| Opsoclonus - Myoclonus Syndrome | |
| Optic neuritis | |
| Palindromic rheumatism (PR) | |
| Pancreatitis | |
| PANDAS (Pediatric Autoimmune | |
| Neuropsychiatric Disorders Associated with | |
| Streptococcus) | |
| Paraneoplastic cerebellar degeneration (PCD) | Anti-Hu (ANNA-1), Anti-Yo, Anti-Tr, anti-amphiphysin |
| Paroxysmal nocturnal hemoglobinuria (PNH) | |
| Parry Romberg syndrome | |
| Pars planitis (peripheral uveitis) | |
| Parsonnage-Turner syndrome | |
| Pemphigoid, Bullous | |
| Pemphigus | |
| Pemphigus foliaceous | |
| Pemphigus Vulgaris | |
| Peripheral neuropathy | |
| Perivenous encephalomyelitis | |
| POEMS syndrome (polyneuropathy, | |
| organomegaly, endocrinopathy, monoclonal | |
| gammopathy, skin changes) | |
| Polyarteritis nodosa | |
| Polychondritis, Relapsing | |
| Polyendocrinopathies, Autoimmune | |
| Polymyalgia Rheumatica | |
| Polymyositis | anti-signal recognition particle, Anti-PM-Scl |
| Polyradiculoneuropathy | |
| Postmyocardial infarction syndrome | |
| Postpericardiotomy syndrome | |
| Poststreptococcal movement disorders, | Lysoganglioside dopamine D2 receptor, Tubulin |
| Sydenham's chorea, and PANDAS | |
| Primary biliary cirrhosis | Antimitochondrial antibodies |
| Primary sclerosing cholangitis | |
| Progesterone dermatitis | |
| Psoriasis | |
| Psoriatic arthritis | |
| Pure red cell aplasia (PRCA) | |
| Pyoderma gangrenosum | |
| Rasmussen encephalitis | GluR3 |
| Raynaud's phenomenon | |
| Reactive Arthritis | |
| Reflex sympathetic dystrophy | |
| Reiter's syndrome | autoimmune response involving cross-reactivity of bacterial |
| | antigens with joint tissues or by bacterial antigens that have |
| | somehow become deposited in the joints |
| Relapsing polychondritis | |
| Restless legs syndrome (RLS) | |
| Retroperitoneal fibrosis | |
| Rheumatic Fever | M proteins of Streptococcus pyogenes |
| Rheumatoid Arthritis | Fc portion of IgG, anti-cyclic citrullinated peptide (Anti-CCP), |
| | Rheumatoid factor |
| Sarcoidosis | |
| Schmidt syndrome | |
| Scleritis | |
| Scleroderma | Anti-topoisomerase antibodies |
| Sjogren's syndrome | Anti-La/SS-Bautoantibodies |
| Sperm & testicular autoimmunity | |
| Stiff-person syndrome | GAD, Gephrin, GABA(B) receptor, amphiphysin |
| Still's Disease, Adult Onset | |
| Subacute bacterial endocarditis (SBE) | |
| Susac's syndrome | |
| Sympathetic ophthalmia (SO) | |
| Takayasu's arteritis | |
| Temporal Arteritis | |
| Temporal arteritis/Giant cell arteritis | |
| Thrombocytopenic purpura (TTP) | |
| Thyrotoxicosis | |
| Tolosa-Hunt syndrome (THS) | |
| Transverse myelitis | |
| Ulcerative colitis (UC) | |
| Undifferentiated connective tissue disease | |
| (UCTD) | |
| Uveitis | |
| Uveomeningoencephalitic Syndrome | |
| Vasculitis | |
| Vitiligo | immune system attacking and destroying the melanocytes |

Additional exemplary therapeutic agents for use in the present invention, their functions and examples of clinical uses are provided in Table 3 and Table 4, below.

TABLE 3
Exemplary Therapeutic Agents

| | | | |
|---|---|---|---|
| Therapeutic | Trade name | Function | Examples of clinical use |

Endocrine disorders (hormone deficiencies)

| | | | |
|---|---|---|---|
| Insulin | Humulin, Novolin | Regulates blood glucose, shifts | Diabetes mellitus, |
| | | potassium into cells | diabetic ketoacidosis, |
| | | | hyperkalaemia |
| Insulin human | Exubera | Insulin formulated for inhalation with | Diabetes mellitus |
| inhalation | | faster onset of action | |
| Insulin aspart; | Novolog (aspart), | Insulin analogues with faster onset of | Diabetes mellitus |
| insulin glulisine; | Apidra (glulisine); | action and shorter duration of action | |
| Insulin lispro | Humalog (lispro) | | |
| Isophane insulin | NPH | Insulin protamine crystalline | Diabetes mellitus |
| | | formulation with slower onset of action | |
| | | and longer duration of action | |
| Insulin detemir; | Levemir (detemir), | Insulin analogues with slower onset of | Diabetes mellitus |
| Insulin glargine | Lantus (glargine) | action and longer duration of action | |
| Insulin zinc | Lente, Ultralente | Insulin zinc hexameric complex with | Diabetes mellitus |
| extended | | slower onset of action and longer | |
| | | duration of action | |
| Pramlintide acetate | Symlin | Mechanism unknown; recombinant | Diabetes mellitus, in |
| | | synthetic peptide analogue of human | combination with insulin |
| | | amylin (a naturally occurring | |
| | | neuroendocrine hormone regulating | |
| | | post-prandial glucose control) | |
| Growth hormone | Genotropin, | Anabolic and anticatabolic effector | Growth failure due to |
| (GH), | Humatrope, | | GH deficiency or chronic |
| somatotropin | Norditropin, | | renal insufficiency, |
| | NorlVitropin, | | Prader-Willi syndrome, |
| | Nutropin, Omnitrope, | | Turner syndrome, AIDS |
| | Protropin, Siazen, | | wasting or cachexia with |
| | Serostim, Valtropin | | antiviral therapy |
| Mecasermin | Increlex | Recombinant insulin-like growth factor 1 | Growth failure in |
| | | (IGF1) induces mitogenesis, chondrocyte | children with GH gene |
| | | growth and organ growth, | deletion or severe |
| | | which combine to restore appropriate | primary IGF1 deficiency |
| | | statural growth | |
| Mecasermin | IPlex | Similar to mecasermin; IGF1 bound to | Growth failure in |
| rinfabate | | IGF binding protein 3 (IGFBP3) is | children with GH gene |
| | | thought to keep the hormone inactive | deletion or severe |
| | | until it reaches its target tissues, thereby | primary IGF1 deficiency |
| | | decreasing hypoglycaemia-like side | |
| | | effects | |

Haemostasis and thrombosis

| | | | |
|---|---|---|---|
| Factor VIII | Bioclate, Helixate, | Coagulation factor | Haemophilia A |
| | Kogenate, | | |
| | Recombinate, ReFacto | | |
| Factor IX | Benefix | Coagulation factor | Haemophilia B |
| Antithrombin III | Ihrombate III | Purified human AT-III from pooled | Hereditary AT-III |
| (AT-111) | | plasma inactivates thrombin by forming | deficiency in connection |
| | | a covalent bond between the catalytic | with surgical or |
| | | serine residue of thrombin and an | obstetrical procedures or |
| | | arginine reactive site on AT-III; AT-III | for thromboembolism |
| | | replacement therapy prevents | |
| | | inappropriate blood-clot formation | |
| Protein C | Ceprotin | After activation by the thrombin- | Treatment and |
| concentrate | | thrombomodulin complex, protein C | prevention of venous |
| | | inhibits coagulation factors Va and | thrombosis and purpura |
| | | VIIIa | fulminans in patients |
| | | | with severe hereditary |
| | | | protein C deficiency |

Metabolic enzyme deficiencies

| | | | |
|---|---|---|---|
| β-Gluco- | Cerezyme | Hydrolyzes glucocerebroside to glucose | Gaucher's disease |
| cerebrosidase | | and ceramide | |
| β-Gluco- | Ceredase (purified | Hydrolyzes glucocerebroside to glucose | Gaucher's disease |
| cerebrosidase | from pooled human | and ceramide | |
| | placenta) | | |
| Alglucosidase-α | Myozyme | Degrades glycogen by catalyzing the | Pompe disease (glycogen |
| | | hydrolysis of α-1,4 and α-1,6 glycosidic | storage disease type II) |
| | | linkages of lysosomal glycogen | |
| Laronidase (α-L- | Aldurazyme | Digests endogenous | Hurler and Hurler-Scheie |
| iduronidase) | | glycosaminoglycans (GAGs) within | forms of |
| | | lysosomes, and thereby prevents an | mucopolysaccharidosis I |
| | | accumulation of GAGs that can cause | |
| | | cellular, tissue, and organ dysfunction | |
| Idursulphase | Elaprase | Cleaves the terminal 2-O-sulphate | Mucopolysaccharidosis |
| (Iduronate-2- | | moieties from the GAGs dermatan | II (Hunter syndrome) |
| sulphatase) | | sulphate and heparan sulphate, thereby | |
| | | allowing their digestion and preventing | |
| | | GAG accumulation | |
| Galsulphase | Naglazyme | Cleaves the terminal sulphate from the | Mucopolysaccharidosis |
| | | GAG dermatan sulphate, thereby | VI |
| | | allowing its digestion and preventing | |
| | | GAG accumulation | |
| Agalsidase-β | Fabrazyme | Enzyme that hydrolyzes | Fabry disease; prevents |
| (human α- | | globotriaosylceramide (GL3) and other | accumulation of lipids |
| galactosidase A) | | glycosphingolipids, reducing | that could lead to renal |
| | | deposition of these lipids in capillary | and cardiovascular |
| | | endothelium of the kidney and certain | complications |
| | | other cell | |
| | | types | |

Pulmonary and gastrointestinal-tract disorders

| | | | |
|---|---|---|---|
| α-1-Proteinase | Aralast, Prolastin | Inhibits elastase-mediated destruction | Congenital α-1- |
| inhibitor | | of pulmonary tissue; purified from | antitrypsin deficiency |
| | | pooled human plasma | |
| Lactase | Lactaid | Digests lactose; purified from fungus | Gas, bloating, cramps |
| | | Aspergillus oryzae | and diarrhoea due to |
| | | | inability to digest lactose |
| Pancreatic | Arco-Lase, Cotazym, | Digests food (protein, fat and | Cystic fibrosis, chronic |
| enzymes (lipase, | Creon, Donnazyme, | carbohydrate); purified from hogs and | pancreatitis, pancreatic |
| amylase, protease) | Pancrease, Viokase, | pigs | insufficiency, post- |
| | Zymase | | Billroth II gastric bypass |
| | | | surgery, pancreatic duct |
| | | | obstruction, steatorrhoea, |
| | | | poor digestion, gas, |
| | | | bloating |

Immunodeficiencies

| | | | |
|---|---|---|---|
| Adenosine | Adagen | Metabolizes adenosine, prevents | Severe combined |
| deaminase | | accumulation of adenosine; purified | immunodeficiency due to |
| (pegademase | | from cows | adenosine deaminase |
| bovine, PEG- | | | deficiency |
| ADA) | | | |
| Pooled | Octagam | Intravenous immunoglobulin | Primary |
| immunoglobulins | | preparation | immunodefiencies |

Other

| | | | |
|---|---|---|---|
| Human albumin | Albumarc, Albumin, | Increases circulating plasma osmolarity, | Decreased production of |
| | Albumiar, AlbuRx, | thereby restoring and maintaining | albumin |
| | Albutein, Flexbumin, | circulating blood volume | (hypoproteinaemia), |
| | Buminate, Plasbumin | | increased loss of albumin |
| | | | (nephrotic syndrome), |
| | | | hypovolaemia, |
| | | | hyperbilirubinaemia |

Cancer

| | | | |
|---|---|---|---|
| Bevacizumab | Avastin | Humanized mAh that binds all isoforms | Colorectal cancer, non- |
| | | ofVEGFA | small-cell lung cancer |
| Cetuximab | Erbitux | Humanized mAh that binds EGFR | Colorectal cancer, head |
| | | | and neck cancer |
| Paniturnumab | Vectibix | Human mAb that binds EGFR | Metastatic colorectal |
| | | | cancer |
| Alemtuzumab | Campath | Humanized mAh directed against CD52 | B-cell chronic |
| | | antigen on T and B cells | lymphocytic leukaemia |
| | | | in patients who have |
| | | | been treated with |
| | | | alkylating agents and |
| | | | who have failed |
| | | | fludabarine therapy |
| Rituximab | Rituxan | Chimeric (human/mouse) mAb that | Relapsed or refractory |
| | | binds CD20, a transmembrane protein | low-grade or follicular |
| | | found on over 90% of B-cell non- | CD20⁺B-cell NHL, |
| | | Hodgkin's lymphomas (NHL); | primary low-grade or |
| | | synergistic effect with some small- | follicular CD20⁺B-cell |
| | | molecule chemotherapeutic agents | NHL in combination |
| | | has been demonstrated in lymphoma | with CVP |
| | | cell lines | chemotherapy; diffuse |
| | | | large B-cell CD20+ |
| | | | NHL in |
| | | | combination with |
| | | | CHOP or other |
| | | | anthracyline- based |
| | | | chemotherapy; |
| | | | rheumatoid arthritis in |
| | | | combination with |
| | | | methotrexate |
| Trastuzumab | Herceptin | Humanized mAb that binds | Breast cancer |
| | | HER2/Neu cell surface receptor and | |
| | | controls cancer | |
| | | cell growth | |

Immunoregulation

| | | | |
|---|---|---|---|
| Abatacept | Orencia | Fusion protein between extracellular | Rheumatoid arthritis |
| | | domain of human CTLA4 and the | (especially when |
| | | modified Fc portion of human | refractory to TNFα |
| | | immunoglobulin G1; selective co- | inhibition) |
| | | stimulation modulator; inhibits T-cell | |
| | | activation by binding to CD80 and | |
| | | CD86, thereby blocking interaction | |
| | | with CD28 and inhibiting autoimmune | |
| | | T-cell activation | |
| Anakinra | Antril, Kineret | Recombinant interleukin 1 (IL1) | Moderate to severe |
| | | receptor antagonist | active rheumatoid |
| | | | arthritis in adults who |
| | | | have failed one or more |
| | | | disease-modifying |
| | | | antirheumatic drug |
| Adaliniumab | Humira | Human mAb that binds specifically to | Rheumatoid arthritis, |
| | | TNFα and blocks its interaction with | Crohn's disease, |
| | | p55 and p75 cell surface TNF receptors, | ankylosing spondylitis, |
| | | resulting in decreased levels of | psoriatic arthritis |
| | | inflammation markers including CRP, | |
| | | ESR, and IL6 | |
| Etanercept | Enbrel | Dimeric fusion protein between | Rheumatoid arthritis, |
| | | recombinant soluble TNF receptor and | polyarticular-course |
| | | Fc portion of human immunoglobulin | juvenile rheumatoid |
| | | G1 | arthritis, psoriatic |
| | | | arthritis, ankylosing |
| | | | spondylitis, plaque |
| | | | psoriasis |
| Infliximab | Remicade | Chimeric mAb that binds and | Rheumatoid arthritis, |
| | | neutralizes TNFα, preventing induction | Crohn's disease, |
| | | of pro-inflammatory cytokines, changes | ankylosing spondylitis, |
| | | in endothelial permeability, activation | psoriatic arthritis, plaque |
| | | of eosinophils and neutrophils, | psoriasis |
| | | induction of acute phase reactants, and | |
| | | enzyme elaboration by synoviocytes | |
| | | and/or chondrocytes | |
| Alefacept | Amevive | Dimeric fusion protein that binds CD2 | Adults with moderate to |
| | | on the surface of lymphocytes and | severe chronic plaque |
| | | inhibits interaction with EFA3; this | psoriasis who are |
| | | association is important for the | candidates for systemic |
| | | activation of T lymphocytes in | therapy or phototherapy |
| | | psoriasis | |
| Efalizumab | Raptiva | Humanized mAb directed against | Adults with chronic |
| | | CD11a | moderate to severe |
| | | | plaque psoriasis who |
| | | | are |
| | | | candidates for systemic |
| | | | therapy or phototherapy |
| Natalizumab | Tysabri | Mechanism unknown; humanized | Relapsing multiple |
| | | mAb that binds to the α4-subunit of | sclerosis |
| | | α4β1 and α4β7 integrins, blocking | |
| | | their interactions with VCAM1 and | |
| | | MadCAM1, respectively | |
| Eculizumab | Soliris | Humanized mAb that binds | Paroxysmal nocturnal |
| | | complement protein C5 and inhibits | haemoglobinuria |
| | | its cleavage to C5a and C5b, | |
| | | preventing the formation of the | |
| | | terminal | |
| | | complement complex C5b-9 | |

Enzymatic degradation of macromolecules

| | | | |
|---|---|---|---|
| Botulinum toxin | Botox | Cleaves SNAP25 at neuromuscular | Many types of dystonia, |
| type A | | junctions to disrupt SNARE complex | particularly cervical; |
| | | and prevent acetylcholine release, | cosmetic uses |
| | | causing flaccid paralysis | |
| Botulinum toxin | Myoblock | Cleaves synaptobrevin at | Many types of dystonia, |
| type B | | neuromuscular junctions to disrupt | particularly cervical; |
| | | SNARE complex and prevent | cosmetic uses |
| | | acetylcholine release, causing flaccid | |
| | | paralysis | |
| Collagenase | Collagenase, Santyl | Collagenase obtained from fermentation | Debridement of chronic |
| | | by Clostridium histolyticum; digests | dermal ulcers and |
| | | collagen in necrotic base of wounds | severely burned areas |
| Human deoxy- | Pulmozyme | Degrades DNA in purulent pulmonary | Cystic fibrosis; decreases |
| ribonuclease I, | | secretions | respiratory tract |
| dornase-α | | | infections in selected |
| | | | patients with FVC |
| | | | greater than 40% of |
| | | | predicted |
| Hyaluronidase | Amphadase (bovine), | Catalyses the hydrolysis of hyaluronic | Used as an adjuvant to |
| (bovine, ovine) | Hydase (bovine), | acid to increase tissue permeability and | increase the absorption |
| | Vitrase (ovine) | allow faster drug absorption | and dispersion of |
| | | | injected drugs, |
| | | | particularly anaesthetics |
| | | | in ophthalmic surgery |
| | | | and certain imaging |
| | | | agents |
| Hyaluronidase | Hylenex | Catalyses the hydrolysis of hyaluronic | Used as an adjuvant to |
| (recombinant | | acid to increase tissue permeability and | increase the absorption |
| human) | | allow faster drug absorption | and dispersion of |
| | | | injected drugs, |
| | | | particularly anaesthetics |
| | | | in ophthalmic surgery |
| | | | and certain imaging |
| | | | agents |
| Papain | Accuzyme, Panafil | Protease from the Carica papaya fruit | Debridement of necrotic |
| | | | tissue or liquefication of |
| | | | slough in acute and |
| | | | chronic lesions, such as |
| | | | pressure ulcers, varicose |
| | | | and diabetic ulcers, |
| | | | burns, postoperative |
| | | | wounds, pilonidal cyst |
| | | | wounds, carbuncles, and |
| | | | other wounds |

Enzymatic degradation of small-molecule metabolites

| | | | |
|---|---|---|---|
| L-Asparaginase | ELSPAR | Provides exogenous asparaginase | Acute lymphocytic |
| | | activity, removing available asparagine | leukaemia, which |
| | | from serum; purified from Escherichia | requires exogenous |
| | | coli | asparagine for |
| | | | proliferation |
| Peg-asparaginase | Oncaspar | Provides exogenous asparaginase | Acute lymphocytic |
| | | activity, removing available asparagine | leukaemia, which |
| | | from serum; purified from E. coli | requires exogenous |
| | | | asparagine for |
| | | | proliferation |
| Rasburicase | Elitek | Catalyzes enzymatic oxidation of uric | Paediatric patients with |
| | | acid into an inactive, soluble metabolite | leukaemia, lymphoma, |
| | | (allantoin); originally isolated from | and solid tumours who |
| | | Aspergillus flavus | are undergoing |
| | | | anticancer therapy that |
| | | | may cause tumour lysis |
| | | | syndrome |

Haemostasis and thrombosis

| | | | |
|---|---|---|---|
| Lepirudin | Refludan | Recombinant hirudin, a thrombin | Heparin-induced |
| | | inhibitor from the salivary gland of the | thrombocytopaenia |
| | | medicinal leech Hirudo medicinalis | |
| Bivalirudin | Angiomax | Synthetic hirudin analogue; specifically | Reduce blood-clotting |
| | | binds both the catalytic site and the | risk in coronary |
| | | anion-binding exosite of circulating and | angioplasty and heparin- |
| | | clot-bound thrombin | induced |
| | | | thrombocytopaenia |
| Streptokinase | Streptase | Converts plasminogen to plasmin; | Acute evolving |
| | | produced by group C β-haemolytic | transmural myocardial |
| | | streptococci | infarction, pulmonary |
| | | | embolism, deep vein |
| | | | thrombosis, arterial |
| | | | thrombosis or embolism, |
| | | | occlusion of |
| | | | arteriovenous cannula |
| Anistreplase | Eminase | Converts plasminogen to plasmin; p- | Thrombolysis in patients |
| (anisoylated | | anisoyl group protects the catalytic | with unstable angina |
| plasminogen | | centre of the plasminogen-streptokinase | |
| streptokinase | | complex and prevents premature | |
| activator complex; | | deactivation, thereby providing longer | |
| APSAC) | | duration of action than streptokinase | |

Haemostasis and thrombosis

| | | | |
|---|---|---|---|
| Alteplase (tissue | Activase | Promotes fibrinolysis by binding fibrin | Pulmonary embolism, |
| plasminogen | | and converting plasminogen to plasmin | myocardial infarction, |
| activator; tPA) | | | acute ischaemic stroke, |
| | | | occlusion of central |
| | | | venous access devices |
| Reteplase (deletion | Retavase | Contains the non-glycosylated kringle | Management of acute |
| mutein of tPA) | | 2 and protease domains of human tPA; | myocardial infarction, |
| | | functions similarly to tPA | improvement of |
| | | | ventricular function |
| Tenecteplase | TNKase | tPA with greater specificity for | Acute myocardial |
| | | plasminogen conversion; has amino- | infarction |
| | | acid substitutions of Thr103 to Asp, | |
| | | Asp117 to Gln, and Ala for amino- | |
| | | acids | |
| | | 296-299 | |
| Urokinase | Abbokinase | Nonrecombinant plasminogen activator | Pulmonary embolism |
| | | derived from human neonatal kidney | |
| | | cells | |
| Factor Vila | NovoSeven | Pro-thrombotic (activated factor VII; | Haemorrhage in |
| | | initiates the coagulation cascade) | patients with |
| | | | haemophilia A or B |
| | | | and inhibitors to factor |
| | | | VIII or factor IX |
| Drotrecogin-α | Xigris | Antithrombotic (inhibits coagulation | Severe sepsis with a |
| (activated protein | | factors Va and VIIIa), anti- | high risk of death |
| C) | | inflammatory | |

Endocrine disorders

| | | | |
|---|---|---|---|
| Salmon calcitonin | Fortical, Miacalcin | Mechanism unknown; inhibits | Postmenopausal |
| | | osteoclast function | osteoporosis |
| Teriparatide | Forteo | Markedly enhances bone formation; | Severe osteoporosis |
| (human | | administered as a once-daily injection | |
| parathyroid | | | |
| hormone residues | | | |
| 1-34) | | | |
| Exenatide | Byetta | Incretin mimetic with actions similar to | Type 2 diabetes resistant |
| | | glucagon-like peptide 1 (GLP1); | to treatment with |
| | | increases glucose-dependent insulin | metformin and a |
| | | secretion, suppresses glucagon | sulphonylurea |
| | | secretion, slows gastric emptying, | |
| | | decreases appetite (first identified in | |
| | | saliva of the Gila monster Heloderma | |
| | | suspectum) | |

Growth Regulation

| | | | |
|---|---|---|---|
| Octreotide | Sandostatin | Potent somatostatin analogue; inhibits | Acromegaly, |
| | | growth hormone, glucagon and insulin | symptomatic relief of |
| | | | VIP-secreting adenoma |
| | | | and metastatic carcinoid |
| | | | tumours |
| Dibotermin-α | Infuse | Mechanism unknown | Spinal fusion surgery, |
| (recombinant | | | bone injury repair |
| human bone | | | |
| morphogenic | | | |
| protein 2; | | | |
| rhBMP2) | | | |
| Recombinant | Osteogenic protein 1 | Mechanism unknown | Tibial fracture nonunion, |
| human bone | | | lumbar spinal fusion |
| morphogenic | | | |
| protein 7 | | | |
| (rhBMP7) | | | |
| Histrelin acetate | Supprelin LA, Vantas | Synthetic analogue of human GnRH; | Precocious puberty |
| (gonadotropin | | acts as a potent inhibitor of | |
| releasing hormone; | | gonadotropin secretion when | |
| GnRH) | | administered continuously by causing a | |
| | | reversible downregulation of GnRH | |
| | | receptors in the pituitary and | |
| | | desensitizing the pituitary gonadotropes | |
| Palifermin | Kepivance | Recombinant analogue of KGF; | Severe oral mucositis in |
| (keratinocyte | | stimulates keratinocyte growth in skin, | patients undergoing |
| growth factor; | | mouth, stomach and colon | chemotherapy |
| KGF) | | | |
| Becaplermin | Regranex | Promotes wound healing by enhancing | Debridement adjunct for |
| (platelet-derived | | granulation tissue formation and | diabetic ulcers |
| growth factor; | | fibroblast proliferation and | |
| PDGF) | | differentiation | |

Other

| | | | |
|---|---|---|---|
| Trypsin | Granulex | Proteolysis | Decubitus ulcer, varicose |
| | | | ulcer, debridement of |
| | | | eschar, dehiscent wound, |
| | | | sunburn |
| Nesiritide | Natrecor | Recombinant B-type natriuretic peptide | Acute decompensated |
| | | | congestive heart failure |

Transplantation

| | | | |
|---|---|---|---|
| Antithymocyte | Thymoglobulin | Selective depletion of T cells; exact | Acute kidney transplant |
| globulin (rabbit) | | mechanism unknown | rejection, aplastic |
| | | | anaemia |
| Basiliximab | Simulect | Chimeric (human/mouse) IgG1 that | Prophylaxis against |
| | | blocks cellular immune response in | allograft rejection in |
| | | graft rejection by binding the alpha | renal transplant patients |
| | | chain of CD25 (IL2 receptor) and | receiving an |
| | | thereby inhibiting the IL2-mediated | immunosuppressive |
| | | activation of lymphocytes | regimen including |
| | | | cyclosporine and |
| | | | corticosteroids |
| Daclizumab | Zenapax | Humanized IgG1 mAb that blocks | Prophylaxis against acute |
| | | cellular immune response in graft | allograft rejection in |
| | | rejection by binding the alpha chain of | patients receiving renal |
| | | CD25 (IL2 receptor) and thereby | transplants |
| | | inhibiting the IL2-mediated activation | |
| | | of lymphocytes | |
| Muromonab-CD3 | Orthoclone, OKT3 | Murine mAb that binds CD3 and blocks | Acute renal allograft |
| | | T-cell function | rejection or steroid- |
| | | | resistant cardiac or |
| | | | hepatic allograft |
| | | | rejection |

Pulmonary disorders

| | | | |
|---|---|---|---|
| Omalizumab | Xolair | Humanized mAb that inhibits IgE | Adults and adolescents |
| | | binding to the high-affinity IgE receptor | (at least 12 years old) |
| | | on mast cells and basophils, decreasing | with moderate to severe |
| | | activation of these cells and release of | persistent asthma who |
| | | inflammatory mediators | have a positive skin test |
| | | | or in vitro reactivity to a |
| | | | perennial aeroallergen |
| | | | and whose symptoms are |
| | | | inadequately controlled |
| | | | with inhaled |
| | | | corticosteroids |
| Palivizumab | Synagis | Humanized IgG1 mAb that binds the A | Prevention of respiratory |
| | | antigenic site of the F protein of | syncytial virus infection |
| | | respiratory syncytial virus | in high-risk paediatric |
| | | | patients |

Infectious diseases

| | | | |
|---|---|---|---|
| Enfuvirtide | Fuzeon | 36 amino-acid peptide that inhibits HIV | Adults and children (at |
| | | entry into host cells by binding to the | least 6 years old) with |
| | | HIV envelope protein gp120/gp41 | advanced HIV infection |

Haemostasis and thrombosis

| | | | |
|---|---|---|---|
| Abciximab | ReoPro | Fab fragment of chimeric | Adjunct to aspirin and |
| | | (human/mouse) mAb 7E3 that inhibits | heparin for prevention of |
| | | platelet aggregation by binding to the | cardiac ischaemia in |
| | | glycoprotein IIb/IIIa integrin receptor | patients undergoing |
| | | | percutaneous coronary |
| | | | intervention or patients |
| | | | about to undergo |
| | | | percutaneous coronary |
| | | | intervention with |
| | | | unstable angina not |
| | | | responding to medical |
| | | | therapy |

Endocrine disorders

| | | | |
|---|---|---|---|
| Pegvisomant | Somavert | Recombinant human growth hormone | Acromegaly |
| | | conjugated to PEG; blocks the growth | |
| | | hormone receptor | |

Other

| | | | |
|---|---|---|---|
| Crotalidae | Crofab | Mixture of Fab fragments of IgG that | Crotalidae envenomation |
| polyvalent immune | | bind and neutralize venom toxins of ten | (Western diamondback, |
| Fab (ovine) | | clinically important North American | Eastern diamondback |
| | | Crotalidae snakes | and Mojave rattlesnakes, |
| | | | and water moccasins) |
| Digoxin immune | Digifab | Monovalent Fab immunoglobulin | Digoxin toxicity |
| serum Fab (ovine) | | fragment obtained from sheep | |
| | | immunized with a digoxin derivative | |
| Ranibizumab | Lucentis | Humanized mAb fragment that binds | Neovascular age-related |
| | | isoforms of vascular endothelial growth | macular degeneration |
| | | factor A (VEGFA) | |

In vivo infectious disease diagnostics

| | | | |
|---|---|---|---|
| Recombinant | DPPD | Noninfectious protein from | Diagnosis of tuberculosis |
| purified protein | | Mycobacterium tuberculosis | exposure |
| derivative (DPPD) | | | |

Hormones

| | | | |
|---|---|---|---|
| Glucagon | GlucaGen | Pancreatic hormone that increases blood | Diagnostic aid to slow |
| | | glucose by stimulating the liver to | gastrointestinal motility |
| | | convert glycogen to glucose | in radiographic studies; |
| | | | reversal of |
| | | | hypoglycaemia |
| Growth hormone | Geref | Recombinant fragment of GHRH that | Diagnosis of defective |
| releasing hormone | | stimulates growth hormone release by | growth-hormone |
| (GHRH) | | somatotroph cells of the pituitary gland | secretion |
| Secretin | ChiRhoStim (human | Stimulation of pancreatic secretions and | Aid in the diagnosis of |
| | peptide), SecreFlo | gastrin | pancreatic exocrine |
| | (porcine peptide) | | dysfunction or |
| | | | gastrinoma; facilitates |
| | | | identification of the |
| | | | ampulla of Vater and |
| | | | accessory papilla during |
| | | | endoscopic retrograde |
| | | | cholangiopancreatography |
| Thyroid stimulating | Thyrogen | Stimulates thyroid epithelial cells or | Adjunctive diagnostic for |
| hormone (TSH), | | well-differentiated thyroid cancer tissue | serum thyroglobulin |
| thyrotropin | | to take up iodine and produce and secrete | testing in the follow-up of |
| | | thyroglobulin, triiodothyronine and | patients with well- |
| | | thyroxine | differentiated thyroid |
| | | | cancer |

Imaging agents, cancer

| | | | |
|---|---|---|---|
| Capromab | ProstaScint | Imaging agent; indium-111-labelled | Prostate cancer detection |
| pendetide | | anti-PSA antibody; recognizes | |
| | | intracellular PSA | |
| Indium-111- | OctreoScan | Imaging agent; indium-111-labelled | Neuroendocrine tumour |
| octreotide | | octreotide | and lymphoma detection |
| Satumomab | OncoScint | Imaging agent; indium-111-labelled | Colon and ovarian cancer |
| pendetide | | mAb specific for tumour-associated | detection |
| | | glycoprotein (TAG-72) | |
| Arcitumomab | CEA-scan | Imaging agent; technetium-labelled | Colon and breast cancer |
| | | anti-CEA antibody | detection |
| Nofetumomab | Verluma | Imaging agent; technetium-labelled | Small-cell lung cancer |
| | | antibody specific for small-cell lung | detection and staging |
| | | cancer | |

Imaging agents, other

| | | | |
|---|---|---|---|
| Apcitide | Acutect | Imaging agent; technetium-labelled | Imaging of acute venous |
| | | synthetic peptide; binds GPIIb/IIIa | thrombosis |
| | | receptors on activated platelets | |
| Imciromab | Myoscint | Imaging agent; indium-111-labelled | Detects presence and |
| pentetate | | antibody specific for human cardiac | location of myocardial |
| | | myosin | injury in patients with |
| | | | suspected myocardial |
| | | | infarction |
| Technetium | NeutroSpec | Imaging agent; technetium-labelled | Diagnostic agent (used in |
| fanolesomab | | anti-CD15 antibody; binds neutrophils | patients with equivocal |
| | | that infiltrate sites of infection | signs and symptoms of |
| | | | appendicitis) |

Examples of in vitro diagnostics

| | | | |
|---|---|---|---|
| HIV antigens | Enzyme immunoassay, | Detects human antibodies to HIV | Diagnosis of HIV |
| | OraQuick, Uni-Gold | (enzyme immunoassay, western blot) | infection |
| Hepatitis C | Recombinant immuno- | Detects human antibodies to hepatitis C | Diagnosis of hepatitis C |
| antigens | blot assay (RIBA) | virus | exposure |
| Deninleukin | Ontak | Directs the cytocidal action of | Persistent or recurrent |
| diftitox | | diphtheria toxin to cells expressing the | cutaneous T-cell |
| | | IL2 receptor | lymphoma whose |
| | | | malignant cells express |
| | | | the CD25 component of |
| | | | the IL2 receptor |
| Ibritumomab | Zevalin | A mAh portion that recognizes | Relapsed or refractory |
| tiuxetan | | CD20+ B cells and induces apoptosis | low-grade, follicular, or |
| | | while the chelation site allows either | transformed B-cell non- |
| | | imaging (In- | Hodgkin's lymphoma |
| | | 111) or cellular damage by beta | (NHL), including |
| | | emission (Y-90) | rituximab-refractory |
| | | | follicular NHL |
| Gemtuzumab | Mylotarg | Humanized anti-CD33 IgG4K mAb | Relapsed CD33+ acute |
| ozogamicin | | conjugated to calicheamicin, a small- | myeloid leukaemia in |
| | | molecule chemotherapeutic agent | patients who are more |
| | | | than 60 years old and are |
| | | | not candidates for |
| | | | cytotoxic chemotherapy |
| Tositumomab | Bexxar, Bexxar I-131 | Tositumomab is a mAb that binds | CD20+ follicular NHL, |
| and | | CD20 surface antigen and stimulates | with and without |
| 131I- | | apoptosis. Tositumomab coupled to | transformation, in patients |
| tositumomab | | radioactive iodine-131 binds CD20 | whose disease is |
| | | surface antigen and delivers cytotoxic | refractory to rituximab |
| | | radiation | and has relapsed |
| | | | following chemotherapy; |
| | | | tositumomab and |
| | | | then131I-tositumomab are |
| | | | used sequentially in the |
| | | | treatment regimen |

Protecting against a deleterious foreign agent (IIIa)

| | | | |
|---|---|---|---|
| Hepatitis B surface | Engerix, Recombivax | Non-infectious protein on surface of | Hepatitis B vaccination |
| antigen (HBsAg) | HB | hepatitis B virus | |
| HPV vaccine | Gardasil | Quadrivalent HPV recombinant vaccine | Prevention of HPV |
| | | (strains 6, 11, 16, 18); contains major | infection |
| | | capsid proteins from four HPV strains | |
| OspA | LYMErix | Non-infectious lipoprotein on outer | Lyme disease |
| | | surface of Borrelia burgdorferi | vaccination |

Treating an autoimmune disease (IIIb)

| | | | |
|---|---|---|---|
| Anti-Rhesus (Rh) | Rhophylac | Neutralizes Rh antigens that could | Routine antepartum and |
| immunoglobulin G | | otherwise elicit anti-Rh antibodies in an | postpartum prevention of |
| | | Rh-negative individual | Rh(D) immunization in |
| | | | Rh(D)-negative women; |
| | | | Rh prophylaxis in case of |
| | | | obstetric complications |
| | | | or invasive procedures |
| | | | during pregnancy; |
| | | | suppression of Rh |
| | | | immunization in Rh(D)- |
| | | | negative individuals |
| | | | transfused with Rh(D)- |
| | | | positive red blood cells |

TABLE 4
Exemplary Theapeutic Agents

| | | | Highest Dev. | |
|---|---|---|---|---|
| Drug ID | Drug Name | CAS Number | Phase | Indication |
| 800006154 | Fingolimod | 162359-56-0 | Marketed | Multiple sclerosis, Chronic inflammatory |
| | | | | demyelinating polyradiculoneuropathy, |
| | | | | Amyotrophic lateral sclerosis, Renal |
| | | | | transplant rejection, Optic neuritis, Type 1 |
| | | | | diabetes mellitus, Rheumatoid arthritis, Graft- |
| | | | | versus-host disease, Myocarditis |
| 800031108 | Guselkumab | 1350289-85-8 | Phase III | Plaque psoriasis, Erythrodermic psoriasis, |
| | | | | Palmoplantar pustulosis, Rheumatoid arthritis, |
| | | | | Psoriatic arthritis |
| 800004275 | Rituximab | 174722-31-7 | Marketed | Non-Hodgkin's lymphoma, Rheumatoid |
| | | | | arthritis, Microscopic polyangiitis, Wegener's |
| | | | | granulomatosis, Follicular lymphoma, Chronic |
| | | | | lymphocytic leukaemia, Nephrotic syndrome, |
| | | | | Lymphoproliferative disorders, Diffuse large |
| | | | | B cell lymphoma, Pemphigus vulgaris, |
| | | | | Transplant rejection, Neuromyelitis optica, |
| | | | | Mantle-cell lymphoma, B cell lymphoma, |
| | | | | Multiple sclerosis, Ulcerative colitis, Sjogren's |
| | | | | syndrome, Ocular inflammation, Scleritis, |
| | | | | Primary biliary cirrhosis, Lupus nephritis, |
| | | | | Systemic lupus erythematosus, Graft-versus- |
| | | | | host disease, Dermatomyositis, Immune |
| | | | | thrombocytopenic purpura |
| 800033563 | Ozanimod | 1306760-87-1 | Phase III | Multiple sclerosis, Ulcerative colitis, Crohn's |
| | | | | disease |
| 800029879 | Corticotropin | 9002-60-2 | Marketed | Membranous glomerulonephritis, Juvenile |
| | gel - | | | rheumatoid arthritis, Polymyositis, Infantile |
| | Mallinckrodt | | | spasms, Rheumatoid arthritis, Adrenal cortex |
| | | | | disorders, Nephrotic syndrome, Sarcoidosis, |
| | | | | Systemic lupus erythematosus, Psoriatic |
| | | | | arthritis, Ankylosing spondylitis, Multiple |
| | | | | sclerosis, Diabetic nephropathies, |
| | | | | Amyotrophic lateral sclerosis |
| 800015868 | Piclidenoson | 152918-18-8 | Phase II/III | Psoriasis, Rheumatoid arthritis, Glaucoma, |
| | | | | Uveitis, Osteoarthritis, Dry eyes, Colorectal |
| | | | | cancer, Solid tumours |
| 800006080 | Eculizumab | 219685-50-4 | Marketed | Paroxysmal nocturnal haemoglobinuria, |
| | | | | Haemolytic uraemic syndrome, Myasthenia |
| | | | | gravis, Neuromyelitis optica, Delayed graft |
| | | | | function, Renal transplant rejection, Guillain- |
| | | | | Barre syndrome, Heart transplant rejection, |
| | | | | Antiphospholipid syndrome, Rheumatoid |
| | | | | arthritis, Autoimmune haemolytic anaemia, |
| | | | | Age-related macular degeneration, |
| | | | | Membranous glomerulonephritis, |
| | | | | Glomerulonephritis, Systemic lupus |
| | | | | erythematosus, Allergic asthma, Motor |
| | | | | neuron disease, Lupus nephritis, Psoriasis, |
| | | | | Dermatomyositis, Bullous pemphigoid, Adult |
| | | | | respiratory distress syndrome, Immune |
| | | | | thrombocytopenic purpura |
| 800019064 | Ocrelizumab | 637334-45-3 | Preregistration | Multiple sclerosis, Systemic lupus |
| | | | | erythematosus, Rheumatoid arthritis, Lupus |
| | | | | nephritis, Haematological malignancies, Eye |
| | | | | disorders |
| 800002822 | Abatacept | 332348-12-6 | Marketed | Rheumatoid arthritis, Juvenile rheumatoid |
| | | | | arthritis, Lupus nephritis, Psoriatic arthritis, |
| | | | | Sjogren's syndrome, Diffuse scleroderma, |
| | | | | Nephrotic syndrome, Inflammation, |
| | | | | Ulcerative colitis, Crohn's disease, Systemic |
| | | | | lupus erythematosus, Multiple sclerosis, |
| | | | | Psoriasis, Graft-versus-host disease, |
| | | | | Transplant rejection, Xenotransplant |
| | | | | rejection |
| 800027858 | Sarilumab | 1189541-98-7 | Preregistration | Rheumatoid arthritis, Juvenile rheumatoid |
| | | | | arthritis, Uveitis, Ankylosing spondylitis |
| 800026523 | Sirukumab | 1194585-53-9 | Preregistration | Rheumatoid arthritis, Giant cell arteritis, |
| | | | | Lupus nephritis, Asthma, Major depressive |
| | | | | disorder, Atherosclerosis |
| 800029418 | Ixekizumab | 1143503-69-8 | Marketed | Plaque psoriasis, Psoriatic arthritis, Pustular |
| | | | | psoriasis, Erythrodermic psoriasis, |
| | | | | Spondylarthritis, Ankylosing spondylitis, |
| | | | | Rheumatoid arthritis |
| 800014900 | Belimumab | 356547-88-1 | Marketed | Systemic lupus erythematosus, Anti- |
| | | | | neutrophil cytoplasmic antibody-associated |
| | | | | vasculitis, Lupus nephritis, Myositis, |
| | | | | Myasthenia gravis, Sjogren's syndrome, |
| | | | | Systemic scleroderma, Renal transplant |
| | | | | rejection, Membranous glomerulonephritis, |
| | | | | Waldenstrom's macroglobulinaemia, |
| | | | | Rheumatoid arthritis |
| 800036998 | 122 0551 | | Phase III | Plaque psoriasis |
| 800023920 | Secukinumab | 1229022-83-6 | Marketed | Plaque psoriasis, Psoriatic arthritis, |
| | | | | Ankylosing spondylitis, Pustular psoriasis, |
| | | | | Rheumatoid arthritis, Psoriasis, Atopic |
| | | | | dermatitis, Alopecia areata, Uveitis, Asthma, |
| | | | | Multiple sclerosis, Dry eyes, Polymyalgia |
| | | | | rheumatica, Type 1 diabetes mellitus, Crohn's |
| | | | | disease |
| 800019919 | Apremilast | 608141-41-9 | Marketed | Psoriatic arthritis, Plaque psoriasis, Behcet's |
| | | | | syndrome, Ankylosing spondylitis, Atopic |
| | | | | dermatitis, Ulcerative colitis, Crohn's disease, |
| | | | | Rheumatoid arthritis, Asthma, Cancer |
| 800037410 | ABT 494 | | Phase III | Rheumatoid arthritis, Crohn's disease, |
| | | | | Ulcerative colitis, Atopic dermatitis |
| 800002909 | Daclizumab | 152923-56-3 | Marketed | Renal transplant rejection, Multiple sclerosis, |
| | | | | Graft-versus-host disease, Asthma, Type 1 |
| | | | | diabetes mellitus, Immune-mediated uveitis, |
| | | | | Liver transplant rejection, Ulcerative colitis, |
| | | | | Psoriasis, Tropical spastic paraparesis, |
| | | | | Haematological malignancies |
| 800035644 | Infliximab | | Marketed | Rheumatoid arthritis, Ulcerative colitis, |
| | biosimilar - | | | Plaque psoriasis, Crohn's disease, |
| | Celltrion | | | Ankylosing |
| | | | | spondylitis, Psoriatic arthritis |
| 800035561 | Adalimumab | 331731-18-1 | Phase III | Rheumatoid arthritis, Plaque psoriasis, |
| | biosimilar - | | | Crohn's disease |
| | Boehringer | | | |
| | Ingelheim | | | |
| 800013731 | Immune globulin - | 9007-83-4 | Marketed | Mucocutaneous lymph node syndrome, |
| | CSL Behring | | | Immune thrombocytopenic purpura, |
| | | | | Immunodeficiency disorders, Guillain-Barre |
| | | | | syndrome, Haemolytic disease of newborn, |
| | | | | Rabies, Hepatitis A, Varicella zoster virus |
| | | | | infections, Chronic inflammatory |
| | | | | demyelinating polyradiculoneuropathy, |
| | | | | Tetanus, Hepatitis B, Encephalitis, Renal |
| | | | | transplant rejection, Skin and soft tissue |
| | | | | infections, Motor neuron disease, Systemic |
| | | | | lupus erythematosus |
| 800032143 | Desoximetasone | 382-67-2 | Marketed | Plaque psoriasis, Atopic dermatitis |
| | topical - Taro | | | |
| | Pharmaceuticals | | | |
| 800029381 | Siponimod | 1220909-40-9 | Phase III | Multiple sclerosis, Polymyositis, |
| | | | | Dermatomyositis, Renal failure, Liver failure |
| 800010359 | Tocilizumab | 375823-41-9 | Marketed | Rheumatoid arthritis, Juvenile rheumatoid |
| | | | | arthritis, Giant lymph node hyperplasia, |
| | | | | Giant cell arteritis, Systemic scleroderma, |
| | | | | Vasculitis, Polymyalgia rheumatica, |
| | | | | Polymyositis, Amyotrophic lateral sclerosis, |
| | | | | Dermatomyositis, Chronic lymphocytic |
| | | | | leukaemia, Ankylosing spondylitis, Multiple |
| | | | | myeloma, Crohn's disease, Pancreatic cancer, |
| | | | | Systemic lupus erythematosus |
| 800018021 | Ofatumumab | 679818-59-8 | Marketed | Chronic lymphocytic leukaemia, Follicular |
| | | | | lymphoma, Multiple sclerosis, Diffuse large |
| | | | | B cell lymphoma, MALT lymphoma, |
| | | | | Neuromyelitis optica, Pemphigus vulgaris, |
| | | | | Rheumatoid arthritis, Waldenstrom's |
| | | | | macroglobulinaemia |
| 800010315 | Mepolizumab | 196078-29-2 | Marketed | Asthma, Chronic obstructive pulmonary |
| | | | | disease, Churg-Strauss syndrome, |
| | | | | Hypereosinophilic syndrome, Nasal polyps, |
| | | | | Eosinophilic oesophagitis |
| 800035998 | Risankizumab | 1612838-76-2 | Phase III | Plaque psoriasis, Crohn's disease, |
| | | | | Ankylosing |
| | | | | spondylitis, Asthma, Psoriatic arthritis, |
| | | | | Psoriasis |
| 800019706 | Dimethyl | 624-49-7 | Marketed | Multiple sclerosis, Rheumatoid arthritis, |
| | fumarate | | | Psoriasis |
| 800008414 | Adalimumab | 331731-18-1 | Marketed | Juvenile rheumatoid arthritis, Ulcerative |
| | | | | colitis, Plaque psoriasis, Ankylosing |
| | | | | spondylitis, Crohn's disease, Hidradenitis |
| | | | | suppurativa, Psoriatic arthritis, |
| | | | | Spondylarthritis, Behcet's syndrome, |
| | | | | Rheumatoid arthritis, Uveitis, Pustular |
| | | | | psoriasis, Unspecified, Interstitial cystitis |
| 800017051 | Calcipotriol/ | | Marketed | Plaque psoriasis, Psoriasis |
| | betamethasone | | | |
| | dipropionate | | | |
| 800030194 | Tildrakizumab | 1326244-10-3 | Phase III | Plaque psoriasis, Autoimmune disorders |
| 800020727 | Golimumab | 476181-74-5 | Marketed | Psoriatic arthritis, Rheumatoid arthritis, |
| | | | | Ankylosing spondylitis, Ulcerative colitis, |
| | | | | Juvenile rheumatoid arthritis, Hearing |
| | | | | disorders, Type 1 diabetes mellitus, |
| | | | | Sarcoidosis, Asthma, Uveitis, Cardiovascular |
| | | | | disorders |
| 800028075 | Brodalumab | 1174395-19-7 | Marketed | Psoriatic arthritis, Erythrodermic psoriasis, |
| | | | | Pustular psoriasis, Plaque psoriasis, Asthma, |
| | | | | Crohn's disease, Rheumatoid arthritis, |
| | | | | Psoriasis |
| 800010395 | Certolizumab | 428863-50-7 | Marketed | Rheumatoid arthritis, Ankylosing spondylitis, |
| | pegol | | | Crohn's disease, Psoriatic arthritis, |
| | | | | Spondylitis, Plaque psoriasis, Juvenile |
| | | | | rheumatoid arthritis, Interstitial cystitis, |
| | | | | Cognition disorders |
| 800027760 | Forigerimod | 497156-60-2 | Phase III | Systemic lupus erythematosus |
| 800029638 | Masitinib | 790299-79-5 | Preregistration | Amyotrophic lateral sclerosis, Mastocytosis, |
| | | | | Prostate cancer, Alzheimer's disease, |
| | | | | Colorectal cancer, Malignant melanoma, |
| | | | | Pancreatic cancer, Gastrointestinal stromal |
| | | | | tumours, Multiple myeloma, Asthma, |
| | | | | Peripheral T-cell lymphoma, Multiple |
| | | | | sclerosis, Crohn's disease, Ovarian cancer, |
| | | | | Progressive supranuclear palsy, Breast |
| | | | | cancer, Chronic obstructive pulmonary |
| | | | | disease, Non- small cell lung cancer, Mood |
| | | | | disorders, Head and neck cancer, |
| | | | | Glioblastoma, Hepatocellular carcinoma, |
| | | | | Gastric cancer, Oesophageal |
| | | | | cancer, Stroke, Psoriasis, Rheumatoid |
| | | | | arthritis |
| 800020410 | Canakinumab | 914613-48-2 | Marketed | Cryopyrin-associated periodic syndromes, |
| | | | | Familial Mediterranean fever, Juvenile |
| | | | | rheumatoid arthritis, Gouty arthritis, |
| | | | | Peroxisomal disorders, Familial autosomal |
| | | | | dominant periodic fever, Cardiovascular |
| | | | | disorders, Behcet's syndrome, Peripheral |
| | | | | arterial occlusive disorders, Mucocutaneous |
| | | | | lymph node syndrome, Abdominal aortic |
| | | | | aneurysm, Pulmonary sarcoidosis, |
| | | | | Atherosclerosis, Osteoarthritis, Diabetic |
| | | | | retinopathy, Chronic obstructive pulmonary |
| | | | | disease, Type 2 diabetes mellitus, |
| | | | | Rheumatoid arthritis, Type 1 diabetes |
| | | | | mellitus, |
| | | | | Polymyalgia rheumatica, Asthma |
| 800032685 | Filgotinib | 1206161-97-8 | Phase III | Rheumatoid arthritis, Crohn's disease, |
| | | | | Ulcerative colitis |
| 800036014 | Etanercept | 185243-69-0 | Phase III | Plaque psoriasis, Rheumatoid arthritis |
| | biosimilar - | | | |
| | Coherus | | | |
| | Biosciences | | | |
| 800001292 | Cladribine | 4291-63-8 | Marketed | Lymphoma, Leukaemia, Chronic |
| | | | | lymphocytic leukaemia, Hairy cell |
| | | | | leukaemia, Multiple |
| | | | | sclerosis, Psoriasis, Transplant rejection |
| 800038738 | Adalimumab | 331731-18-1 | Registered | Ankylosing spondylitis, Psoriatic arthritis, |
| | biosimilar - | | | Ulcerative colitis, Juvenile rheumatoid |
| | Amgen | | | arthritis, Rheumatoid arthritis, Crohn's |
| | | | | disease, Plaque psoriasis |
| 800018418 | Ustekinumab | 815610-63-0 | Marketed | Plaque psoriasis, Psoriatic arthritis, Crohn's |
| | | | | disease, Spondylarthritis, Ulcerative colitis, |
| | | | | Systemic lupus erythematosus, Atopic |
| | | | | dermatitis, Inflammation, Palmoplantar |
| | | | | pustulosis, Sarcoidosis, Rheumatoid arthritis, |
| | | | | Primary biliary cirrhosis, Multiple sclerosis |
| 800024855 | Ponesimod | 854107-55-4 | Phase III | Multiple sclerosis, Graft-versus-host disease, |
| | | | | Immunological disorders, Plaque psoriasis |
| 800039480 | Adalimumab | | Phase III | Plaque psoriasis, Rheumatoid arthritis |
| | biosimilar - | | | |
| | Sandoz | | | |
| 800017661 | Teriflunomide | 108605-62-5 | Marketed | Multiple sclerosis |
| 800038193 | Infliximab | | Phase III | Rheumatoid arthritis |
| | biosimilar - | | | |
| | Pfizer | | | |
| 800011618 | Laquinimod | 248281-84-7 | Preregistration | Multiple sclerosis, Huntington's disease, |
| | | | | Crohn's disease, Lupus nephritis, Systemic |
| | | | | lupus erythematosus |
| 800004155 | Infliximab | 170277-31-3 | Marketed | Crohn's disease, Rheumatoid arthritis, |
| | | | | Psoriasis, Ulcerative colitis, Psoriatic |
| | | | | arthritis, Ankylosing spondylitis, Plaque |
| | | | | psoriasis, Behcet's syndrome, |
| | | | | Mucocutaneous lymph |
| | | | | node syndrome, Hepatitis C, Pyoderma, |
| | | | | Berylliosis |
| 800018131 | Baricitinib | 1187594-09-7 | Preregistration | Rheumatoid arthritis, Systemic lupus |
| | | | | erythematosus, Diabetic nephropathies, |
| | | | | Atopic |
| | | | | dermatitis, Psoriasis |
| 800003804 | Glatiramer acetate | 147245-92-9 | Marketed | Multiple sclerosis, Amyotrophic lateral |
| | | | | sclerosis, Huntington's disease, Neurological |
| | | | | disorders, Glaucoma |
| 800027190 | Amifampridine | 54-96-6 | Marketed | Lambert-Eaton myasthenic syndrome, |
| | | | | Congenital myasthenic syndromes, |
| | | | | Myasthenia gravis |
| 800019029 | Tofacitinib | 477600-75-2 | Marketed | Rheumatoid arthritis, Psoriatic arthritis, |
| | | | | Juvenile rheumatoid arthritis, Ulcerative |
| | | | | colitis, Plaque psoriasis, Atopic dermatitis, |
| | | | | Ankylosing spondylitis, Crohn's disease, Dry |
| | | | | eyes, Renal transplant rejection, Irritable |
| | | | | bowel syndrome, Asthma |
| 800038107 | Etanercept | | Registered | Plaque psoriasis, Ankylosing spondylitis, |
| | biosimilar - | | | Psoriatic arthritis, Rheumatoid arthritis, |
| | Sandoz | | | Juvenile rheumatoid arthritis |
| 800043035 | Ulobetasol lotion - | | Phase III | Plaque psoriasis |
| | Valeant | | | |
| | Pharmaceuticals | | | |
| 800037371 | Rituximab | 174722-31-7 | Phase III | Rheumatoid arthritis, Follicular lymphoma |
| | biosimilar - | | | |
| | Boehringer | | | |
| | Ingelheim | | | |
| 800040562 | DFD 06 | | Phase III | Plaque psoriasis |
| 800003273 | Etanercept | 185243-69-0 | Marketed | Juvenile rheumatoid arthritis, Plaque |
| | | | | psoriasis, Ankylosing spondylitis, Psoriatic |
| | | | | arthritis, Rheumatoid arthritis, Graft-versus- |
| | | | | host disease, Discoid lupus erythematosus, |
| | | | | Metabolic syndrome, Heart failure, |
| | | | | Wegener's granulomatosis, Pulmonary |
| | | | | fibrosis, Transplant rejection, Asthma, Adult- |
| | | | | onset |
| | | | | Still's disease, Myasthenia gravis, Behcet's |
| | | | | syndrome, Cachexia, Septic shock |
| 800041067 | Adalimumab | 331731-18-1 | Phase III | Plaque psoriasis |
| | biosimilar - | | | |
| | Coherus | | | |
| | BioSciences | | | |
| 800042069 | Adalimumab | | Phase III | Plaque psoriasis, Rheumatoid arthritis, |
| | biosimilar - | | | Inflammation, Autoimmune disorders |
| | Momenta | | | |
| | Pharmaceuticals | | | |
| 800043884 | Bee venom - | | Marketed | Osteoarthritis, Multiple sclerosis |
| | Apimeds | | | |
| 800035854 | Adalimumab | 331731-18-1 | Phase III | Rheumatoid arthritis |
| | biosimilar - | | | |
| | Fujifilm Kyowa | | | |
| | Kirin | | | |
| | Biologics | | | |
| 800021494 | Anifrolumab | 1326232-46-5 | Phase III | Systemic lupus erythematosus, Scleroderma |
| 800033985 | Tazarotene/ulobetasol | | Phase III | Plaque psoriasis |
| 800029302 | Olokizumab | 1007223-17-7 | Phase III | Rheumatoid arthritis |
| 800002472 | Anakinra | 143090-92-0 | Marketed | Rheumatoid arthritis, Cryopyrin-associated |
| | | | | periodic syndromes, Gout, Juvenile |
| | | | | rheumatoid arthritis, Septic shock, |
| | | | | Ankylosing spondylitis, Osteoarthritis, Graft- |
| | | | | versus-host |
| | | | | disease, Pneumococcal infections |
| 800031049 | Calcipotriol - | 112965-21-6 | Marketed | Plaque psoriasis, Psoriasis |
| | Stiefel | | | |
| 800006904 | Fampridine | 504-24-5 | Marketed | Multiple sclerosis, Neurological disorders, |
| | sustained- release | | | Stroke, Spinocerebellar degeneration, Spinal |
| | | | | cord injuries, Parkinson's disease, Cerebral |
| | | | | palsy |
| 800018689 | Clobetasol | 25122-41-2 | Marketed | Atopic dermatitis, Psoriasis, Skin disorders |
| | propionate topical - | | | |
| | Galderma | | | |
| 800023488 | Prednisone | 53-03-2 | Marketed | Asthma, Rheumatoid arthritis, Chronic |
| | controlled- release - | | | obstructive pulmonary disease, Psoriatic |
| | Horizon | | | arthritis, Ankylosing spondylitis, |
| | Pharma/Vectura | | | Polymyalgia rheumatica, Nocturnal asthma |
| 800007752 | Ciclosporin - | 59865-13-3 | Marketed | Psoriasis, Liver transplant rejection, |
| | Novartis | | | Transplant rejection, Pancreas transplant |
| | | | | rejection, Atopic dermatitis, Rheumatoid |
| | | | | arthritis, Heart transplant rejection, |
| | | | | Myasthenia gravis, Renal transplant |
| | | | | rejection, |
| | | | | Ulcerative colitis |
| 800006793 | Natalizumab | 189261-10-7 | Marketed | Multiple sclerosis, Crohn's disease, Stroke, |
| | | | | Graft-versus-host disease, Rheumatoid |
| | | | | arthritis, Multiple myeloma |
| 800002523 | Alemtuzumab | 216503-57-0 | Marketed | Multiple sclerosis, Chronic lymphocytic |
| | | | | leukaemia, T cell prolymphocytic leukaemia, |
| | | | | Graft-versus-host disease, Rheumatoid |
| | | | | arthritis |
| 800016270 | Atacicept | | Phase II/III | Systemic lupus erythematosus, Rheumatoid |
| | | | | arthritis, Multiple sclerosis, Lupus nephritis, |
| | | | | Chronic lymphocytic leukaemia, Non- |
| | | | | Hodgkin's lymphoma, Multiple myeloma |
| 800038364 | Adalimumab | 331731-18-1 | Phase III | Rheumatoid arthritis |
| | biosimilar - | | | |
| | Pfizer | | | |
| 800038469 | Infliximab | 170277-31-3 | Registered | Rheumatoid arthritis, Ulcerative colitis, |
| | biosimilar - | | | Psoriatic arthritis, Plaque psoriasis, Crohn's |
| | Merck & | | | disease, Ankylosing spondylitis |
| | Co/Samsung | | | |
| | Bioepis | | | |
| 800039191 | DFD 01 | 5593-20-4 | Marketed | Plaque psoriasis |
| 800033254 | Pefcalcitol | 381212-03-9 | Phase III | Plaque psoriasis, Palmoplantar keratoderma |
| 800015135 | Immune globulin | 9007-83-4 | Marketed | Immune thrombocytopenic purpura, |
| | 10% - Grifols | | | Immunodeficiency disorders, Chronic |
| | | | | inflammatory demyelinating |
| | | | | polyradiculoneuropathy, Myasthenia gravis, |
| | | | | Multiple sclerosis |
| 800040965 | ALKS 8700 | | Phase III | Multiple sclerosis |
| 800016064 | Peginterferon | 1211327-92-2 | Marketed | Multiple sclerosis |
| | beta-1a - Biogen | | | |
| 800040608 | Fluocinonide | 356-12-7 | Marketed | Skin disorders, Plaque psoriasis |
| | cream- Valeant | | | |
| 800006422 | Interferonbeta- | 145258-61-3 | Marketed | Multiple sclerosis, Hepatitis B, Human |
| | 1a - Biogen | | | papillomavirus infections, Hepatitis C, |
| | | | | Ulcerative colitis, Glioma, Chronic |
| | | | | inflammatory demyelinating |
| | | | | polyradiculoneuropathy, Pulmonary fibrosis |
| 800000782 | Interferonbeta- | 145155-23-3 | Marketed | Multiple sclerosis, Prostate cancer, |
| | 1b - Bayer | | | Cardiomyopathies, HIV infections, |
| | HealthCare | | | Rhinovirus infections |
| | Pharmaceuticals/ | | | |
| | Novartis | | | |
| 800001086 | Meloxicam | 71125-38-7 | Marketed | Osteoarthritis, Periarthritis, Rheumatoid |
| | | | | arthritis, Neuropathic pain, Gout, Ankylosing |
| | | | | spondylitis, Back pain, Juvenile rheumatoid |
| | | | | arthritis, Preterm labour |
| 800003883 | Alefacept | 222535-22-0 | Marketed | Psoriasis, Transplant rejection, Psoriatic |
| | | | | arthritis |
| 800006795 | Celecoxib | 169590-42-5 | Marketed | Dysmenorrhoea, Acute pain, Tenosynovitis, |
| | | | | Familial adenomatous polyposis, Back pain, |
| | | | | Ankylosing spondylitis, Tendinitis, Dental |
| | | | | pain, Rheumatoid arthritis, Postoperative |
| | | | | pain, Osteoarthritis, Pain, Rheumatic |
| | | | | disorders, Juvenile rheumatoid arthritis, |
| | | | | Cervicobrachial syndrome, Periarthritis, |
| | | | | Non-small cell lung cancer, Stomatitis, |
| | | | | Gouty arthritis, Bladder |
| | | | | cancer, Alzheimer's disease, Prostate cancer |
| 800024954 | Esomeprazole/ | | Marketed | Osteoarthritis, Rheumatoid arthritis, |
| | naproxen | | | Ankylosing spondylitis |
| 800002515 | Tazarotene | 118292-40-3 | Marketed | Acne vulgaris, Psoriasis, Photodamage |
| | topical | | | |
| 800004239 | Calcipotriol | 112965-21-6 | Marketed | Psoriasis |
| 800013806 | Epratuzumab | 205923-57-5 | Phase III | Systemic lupus erythematosus, Acute |
| | | | | lymphoblastic leukaemia, Non-Hodgkin's |
| | | | | lymphoma, Cachexia |
| 800007022 | Interferonbeta- | 145258-61-3 | Marketed | Multiple sclerosis, Hepatitis C, Human |
| | 1a - Merck | | | papillomavirus infections, Non-small cell |
| | Serono | | | lung |
| | | | | cancer, Ulcerative colitis, Crohn's disease, |
| | | | | Rheumatoid arthritis |
| 800045068 | Ulobetasol lotion - | 66852-54-8 | Registered | Plaque psoriasis |
| | Sun Pharmaceutical | | | |
| | Industries | | | |
| 800031664 | Immune globulin | 308067-58-5 | Marketed | Immunodeficiency disorders, Immune |
| | 10% - | | | thrombocytopenic purpura, Chronic |
| | Octapharma | | | inflammatory demyelinating |
| | | | | polyradiculoneuropathy, Alzheimer's disease |
| 800034238 | Methotrexate | 59-05-2 | Marketed | Psoriasis, Rheumatoid arthritis, Juvenile |
| | subcutaneous | | | rheumatoid arthritis |
| | auto-injection - | | | |
| | Antares Pharma | | | |
| 800044876 | VAL BRO 03 | | Phase III | Psoriatic arthritis |
| 800004586 | Acitretin | 55079-83-9 | Marketed | Psoriasis, Dermatitis, Cancer |
| 800044389 | Juvenile | | Phase III | Juvenile rheumatoid arthritis |
| | rheumatoid | | | |
| | arhtritis | | | |
| | therapeutic - | | | |
| | Marathon | | | |
| | Pharmaceuticals | | | |
| 800006246 | Rheumatoid | | Phase III | Rheumatoid arthritis |
| | arthritis | | | |
| | vaccine (IR | | | |
| | 501) - Immune | | | |
| | Response | | | |
| | BioPharma | | | |
| 800025490 | Ibuprofen/ | 1011231-26-7 | Marketed | Musculoskeletal pain, Osteoarthritis, |
| | famotidine | | | Rheumatoid arthritis, NSAID-induced ulcer, |
| | | | | Ankylosing spondylitis |
| 800039732 | Methotrexate | 59-05-2 | Marketed | Juvenile rheumatoid arthritis, Rheumatoid |
| | subcutaneous | | | arthritis, Psoriasis |
| | auto-injection - | | | |
| | Medac Pharma | | | |
| 800009362 | Calcitriol - | 32222-06-3 | Marketed | Plaque psoriasis |
| | Galderma | | | |
| 800014212 | Mometasone/ | | Marketed | Psoriasis, Skin disorders |
| | salicylic acid | | | |
| 800022272 | Clobetasol | 25122-46-7 | Marketed | Atopic dermatitis, Psoriasis |
| | propionate foam | | | |
| | (Olux-E) - | | | |
| | Stiefel | | | |
| | Laboratories | | | |
| 800012485 | Clobetasol | 25122-46-7 | Marketed | Skin disorders, Psoriasis |
| | propionate foam | | | |
| | (Olux) - | | | |
| | Stiefel | | | |
| | Laboratories | | | |
| 800009052 | Mitoxantrone | 65271-80-9 | Marketed | Breast cancer, Acute nonlymphocytic |
| | | | | leukaemia, Cancer, Acute promyelocytic |
| | | | | leukaemia, Cancer pain, Acute myeloid |
| | | | | leukaemia, Ovarian cancer, Leukaemia, Liver |
| | | | | cancer, Multiple sclerosis, Non-Hodgkin's |
| | | | | lymphoma |
| 800012483 | Betamethasone | 2152-44-5 | Marketed | Atopic dermatitis, Psoriasis, Seborrhoeic |
| | valerate foam - | | | dermatitis, Skin disorders |
| | Stiefel | | | |
| | Laboratories | | | |
| 800012233 | Mahonia | | Marketed | Psoriasis |
| | aquifolium extract | | | |

In some embodiments, the therapeutic is an incretin mimetic or derivative of an incretin (e.g. human incretin), such as liraglutide (Victoza®, Saxenda®), semaglutide, exenatide (Byetta®, Bydureon®), or dulaglutide (Trulicity®); or octreotide, calcitonin (including salmon calcitonin), parathyroid hormone (PTH), teriparatide (a recombinant form of PTH) insulin, a peptide agonist of GLP-1 such as exenatide, liraglutide, lixisenatide, albiglutide and/or dulaglutide, a GLP-1/GIP co-agonist, a GLP-2 agonist, or a peptide GPCR agonist.

In some embodiments, the biological molecule is a brain reactive antigen. Examples are provided in Table 5 below.

TABLE 5
Brain Reactive Antigens
Diamond et al., 2015: Brain reactive antibodies and disease

| | | | Ab | | | | | |
|---|---|---|---|---|---|---|---|---|
| | | | useful | | Ab relevant | | Subcellular | |
| | Defined | Ab in | in | Clinical | to disease | | site of | |
| Disorder | antigen | CSF | diagnosis | response | mechanism | Mechanism | action | Etiology |
| HAM/tropical | hnRNP | Yes | ND | ND | ND | Inhibits | Intracellular | Molecular |
| spastic | A1 | (245) | | | | neuronal | | mimicry |
| paraparesis | (244) | | | | | activity | | (246) |
| | | | | | | (246) | | |
| Neuromyelitis | AQP4(150, | Yes | Yes | Yes | Yes (152, | Receptor- | Extracellular | Autoimmunity |
| optica | 151, 171) | (171, | (154) | (248), | 249) | mediated | | |
| | | 247) | | suppression | | internalization; | | |
| | | | | | | complement- | | |
| | | | | | | mediated | | |
| | | | | | | toxicity | | |
| Acute | MOG | Yes | Yes | Yes | Yes (254) | Complement- | Extracellular | Autoimmunity |
| disseminated | (138) | (138, | (250) | (251-253), | | mediated | | |
| encephalomyelitis | | 139) | | modulation | | demyelination | | |
| Systemic | NR2A/ | Yes | Yes | Yes | Yes (2, | Receptor | Extracellular | Autoimmunity |
| lupus | NR2B | (106-108, | (103-108, | (106, | 100, | modulation, | | |
| erythematosus | | 255, 256) | 255, 256) | 107) | 257) | apoptosis | | |
| | | | | | | (50, 100, | | |
| | | | | | | 101) | | |
| | Neuronal | Yes | Yes | ND | Yes (116) | Ca2+ | Extracellular | Autoimmunity |
| | surface P | (114) | (258) | | | influx, | | |
| | antigen | | | | | apoptosis | | |
| | (116) | | | | | (116) | | |
| Poststreptococcal | Lysogan | Yes | Yes | Yes | Yes (213, | Aberrant | Extracellular | Molecular |
| movement | glioside | (199, | | (218) | 214, | cell | | mimicry |
| disorders, | dopamine D2 | 215, | | | 217) | signaling, | | |
| Sydenham's | receptor | 216) | | | | neurotransmitter | | |
| chorea, and | Tubulin | | | | | release | Intracellular | |
| PANDAS | (199, | | | | | (216, 259) | | |
| | 215, 216, | | | | | | | |
| | 259) | | | | | | | |
| Celiac | Synapsin 1 | Yes | ND | Yes | Yes (260) | ND | Intracellular | Autoimmunity/ |
| disease | (260) | | | | | | | molecular |
| | | | | | | | | mimicry |
| | Transglutaminase | ND | Yes | Yes | ND | ND | Intracellular | Autoimmunity |
| | | | (261) | (262) | | | | |
| Autism | ND (238, | ND | ND | ND | ND (239) | ND | ND | ND |
| | 239) | | | (263) | | | | |
| Limbic | AMPAR | Yes | Yes | Yes | Yes | Altered | Extracellular | Autoimmunity |
| encephalitis | (GluR1, | | | | | receptor | | |
| | GluR2) | | | | | location | | |
| | | | | | | (264) | | |
| | NMDAR | Yes | Yes | Yes | Yes | Receptor | Extracellular | Autoimmunity |
| | (265) | | | | | internalization | | |
| | [NR1/NR2B | | | | | (224) | | |
| | (224)] | | | | | | | |
| | Lgi1 (24) | Yes | Yes | Yes (24, | ND | ND (24) | Extracellular | Autoimmunity |
| | | (264) | (24) | 266) | | | | |
| Rasmussen | GluR3 | Yes | Yes | Yes | Yes (269) | Complement- | Extracellular | Autoimmunity |
| encephalitis | (267) | | | (268, | | mediated | | |
| | | | | 269) | | toxicity | | |
| | | | | | | (270) | | |
| Hashimoto's | Aldehyde | Yes | ND | ND | ND | ND | Intracellular | Autoimmunity |
| encephalitis | reductase | (271) | | | | | | |
| | (271) | | | | | | | |
| | Thyroglobulin | | | | | | Extracellular | |
| | (271, 272) | | | | | | | |
| Encephalitis | ND | Yes | ND | ND | ND | ND | ND | Autoimmunity |
| lethargica | | (273) | | | | | | |
| Stiff-person | GAD | Yes | Yes | Yes | Yes (233) | ND | Intracellular | Autoimmunity |
| syndrome | (274) | (275, | (233) | (274) | | | | |
| | | 276) | | | | | | |
| | Gephryin | Yes | Yes | Yes | Yes | ND | Intracellular | Autoimmunity |
| | (275) | | | | | | | |
| | GABA(B) | | | | | | Extracellular | |
| | receptor | | | | | | | |
| | (277) | | | | | | | |
| | Amphiphysin | Yes | Yes | Yes | Yes | Synaptic | Intracellular | Autoimmunity |
| | (233) | | | | | inhibition | | |
| | | | | | | (233) | | |

Other biological molecules for use in making the cargo-loaded milk vesicles described herein can be found in, e.g., WO2018102397 and references cited therein, the relevant disclosures of each of which are incorporated by reference for the purposes or subject matter referenced herein.

In some embodiments, the biological molecule cargo is a peptide. In some embodiments, the peptide is encapsulated in the milk vesicle. In some embodiments the peptide is associated with the milk vesicle. In some embodiments, the peptide cargo associated or encapusulated by the milk vesicle is protected from enzymatic digestion. e.g., by digestive enzymes. In some embodiments, the peptide cargo is protected from acidic conditions in the stomach, peristaltic motions, and/or exposure to the various proteases that break down ingested components in the gastrointestinal tract.

B. Modifications of Cargo

In some embodiments, the cargo loaded into the milk vesicles can be modified, for example, by a hydrophobic moiety to enhance its uptake by the milk vesicle. In some embodiments, the hydrophobic group is selected from a lipid, a sterol, a steroid, a terpene, cholic acid, adamantane acetic acid, 1-pyrene butyric acid, 1,3-bis-O(hexadecyl)glycerol, a geranyloxyhexyl group, hexadecylglycerol, borneol, 1,3-propanediol, heptadecyl group, O3-(oleoyl)lithocholic acid, O3-(oleoyl)cholenic acid, dimethoxytrityl, or phenoxazine.

Any of the biological molecules described herein may be conjugated (e.g., covalently) with a hydrophobic group as also described herein. Examples include iRNA, siRNA, mRNA, DNA, hormone, protein such as an antibody or others described herein, peptidomimetic, or small molecule. In some embodiments, the therapeutic agent is a siRNA modified to comprise a lipid or steroid or other hydrophobic group, such as those described in detail herein, infra. In some embodiments, the hydrophobic group is a fatty acid or a sterol or steroid such as cholesterol.

In some embodiments, the therapeutic agent comprises or is modified to comprise a hydrophobic group selected from a terpene such as nerolidol, farnesol, limonene, linalool, geraniol, carvone, fenchone, or menthol; a lipid such as palmitic acid or myristic acid; cholesterol; oleyl; retinyl; cholesteryl residues; cholic acid; adamantane acetic acid; 1-pyrene butyric acid; dihydrotestosterone; 1,3-Bis-O(hexadecyl)glycerol; geranyloxyhexyl group; hexadecylglycerol; borneol; 1,3-propanediol; heptadecyl group; O3-(oleoyl)lithocholic acid; O3-(oleoyl)cholenic acid; dimethoxytrityl; or phenoxazine. In some embodiments, the hydrophobic group is cholesterol. In some embodiments, the hydrophobic group is a fat-soluble vitamin. In some embodiments, the hydrophobic group is selected from folic acid; cholesterol; a carbohydrate; vitamin A; vitamin E; or vitamin K.

Other hydrophobic groups include, for example, steroids (e.g., uvaol, hecigenin, diosgenin), terpenes (e.g., triterpenes, e.g., sarsasapogenin, friedelin, epifriedelanol derivatized lithocholic acid), vitamins (e.g., folic acid, vitamin A, biotin, pyridoxal, or vitamin E), carbohydrates, proteins, and protein binding agents, as well as lipophilic molecules, e.g., thiol analogs of cholesterol, cholic acid, cholinic acid, lithocholic acid, adamantane acetic acid, 1-pyrene butyric acid, dihydrotestosterone, glycerol (e.g., esters (e.g., mono, bis, or tris fatty acid esters, e.g., C10, C11, C12, C13, C14, C15, C16, C17, C18, C19, or C20 fatty acids) and ethers thereof, e.g., C10, C11, C12, C13, C14, C15, C16, C17, C18, C19, or C20 alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., gyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

In some embodiments, the hydrophobic group is a sterol, steroid, hopanoid, hydroxysteroid, secosteroid, or analog thereof with lipophilic properties. Exemplary sterol moieties include a phytosterol, mycosterol, or zoosterol. Exemplary zoosterols include cholesterol and 24S-hydroxycholesterol; exemplary phytosterols include ergosterol (mycosterol), campesterol, sitosterol, and stigmasterol. In some embodiments, the sterol is selected from ergosterol, 7-dehydrocholesterol, cholesterol, 24S-hydroxycholesterol, lanosterol, cycloartenol, fucosterol, saringosterol, campesterol, β-sitosterol, sitostanol, coprostanol, avenasterol, or stigmasterol. Sterols may be found either as free sterols, acylated (sterol esters), alkylated (steryl alkyl ethers), sulfated (sterol sulfate), or linked to a glycoside moiety (steryl glycosides), which can be itself acylated (acylated sterol glycosides). Exemplary steroid moieties include dihydrotestosterone, uvaol, hecigenin, diosgenin, progesterone, or cortisol.

The hydrophobic moiety may be conjugated to the therapeutic agent at any chemically feasible location, e.g. on a nucleic acid molecule at the 5′ and/or 3′ end (or one or both strands of the nucleic acid molecule, if it is a duplex). In a particular embodiment, the hydrophobic moiety is conjugated only to the 3′ end, more particularly the 3′ end of the sense strand in double stranded molecules. The hydrophobic moiety may be conjugated directly to the nucleic acid molecule or via a linker. The hydrophobic moiety may be adamantane, cholesterol, a steroid, long chain fatty acid, lipid, phospholipid, glycolipid, or derivatives thereof.

For example, sterols may be conjugated to the therapeutic at the available —OH group. Exemplary sterols have the general skeleton shown below:

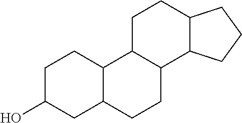 [SMILES: OC1CCC2C(CCC3C4CCCC4CCC23)C1]

As a further example, ergosterol has the structure below:

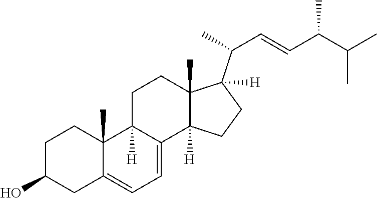

Cholesterol has the structure below:

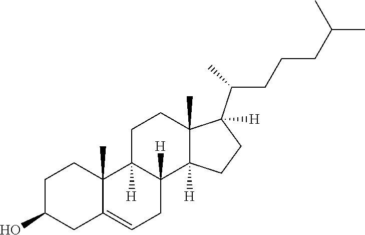

Accordingly, in some embodiments, the free —OH group of a sterol or steroid is used to conjugate the therapeutic to the sterol or steroid.

In some embodiments, the hydrophobic group is a lipid, such as a fatty acid, phosphatide, phospholipid, or analogue thereof (e.g. phophatidylcholine, lecithin, phosphatidylethanolamine, cephalin, or phosphatidylserine or analogue or portion thereof, such as a partially hydrolyzed portion thereof). In some embodiments, the fatty acid is a short-chain, medium-chain, or long-chain fatty acid. In some embodiments, the fatty acid is a saturated fatty acid. In some embodiments, the fatty acid is an unsaturated fatty acid. In some embodiments, the fatty acid is a monounsaturated fatty acid. In some embodiments, the fatty acid is a polyunsaturated fatty acid, such as an ω-3 (omega-3) or ω-6 (omega-6) fatty acid. In some embodiments, the lipid, e.g., fatty acid, has a C₂-C₆₀ chain. In some embodiments, the lipid, e.g., fatty acid, has a C₂-C₂₈ chain. In some embodiments, the lipid, e.g., fatty acid, has a C₂-C₄₀ chain. In some embodiments, the lipid, e.g., fatty acid, has a C₂-C₁₂ or C₄-C₁₂ chain. In some embodiments, the lipid, e.g., fatty acid, has a C₄-C₄₀ chain.

In some embodiments, the therapeutic agent may be modified by two lipids. In some embodiments, the two lipids, e.g. fatty acids, taken together have 6-80 carbon atoms (an equivalent carbon number (ECN) of 6-80), for example, 10-70, 20-60, 30-60, 30-50, or 40-80.

Suitable fatty acids include saturated straight-chain fatty acids, saturated branched fatty acids, unsaturated fatty acids, hydroxy fatty acids, and polycarboxylic acids. In some embodiments, such fatty acids have up to 32 carbon atoms.

Examples of useful saturated straight-chain fatty acids include those having an even number of carbon atoms, such as butyric acid, caproic acid, caprylic acid, capric acid, lauric acid, myristic acid, palmitic acid, stearic acid, arachic acid, behenic acid, lignoceric acid, hexacosanoic acid, octacosanoic acid, triacontanoic acid and n-dotriacontanoic acid, and those having an odd number of carbon atoms, such as propionic acid, n-valeric acid, enanthic acid, pelargonic acid, hendecanoic acid, tridecanoic acid, pentadecanoic acid, heptadecanoic acid, nonadecanoic acid, heneicosanoic acid, tricosanoic acid, pentacosanoic acid, and heptacosanoic acid.

Examples of suitable saturated branched fatty acids include isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, isostearic acid, 17-methyloctadecanoic acid, isoarachic acid, 19-methyl-eicosanoic acid, α-ethyl-hexanoic acid, α-hexyldecanoic acid, α-heptylundecanoic acid, 2-decyltetradecanoic acid, 2-undecyltetradecanoic acid, 2-decylpentadecanoic acid, 2-undecylpentadecanoic acid, and Fine oxocol 1800 acid (product of Nissan Chemical Industries, Ltd.). Suitable saturated odd-carbon branched fatty acids include anteiso fatty acids terminating with an isobutyl group, such as 6-methyl-octanoic acid, 8-methyl-decanoic acid, 10-methyl-dodecanoic acid, 12-methyl-tetradecanoic acid, 14-methyl-hexadecanoic acid, 16-methyl-octadecanoic acid, 18-methyl-eicosanoic acid, 20-methyl-docosanoic acid, 22-methyl-tetracosanoic acid, 24-methyl-hexacosanoic acid, and 26-methyloctacosanoic acid.

Examples of suitable unsaturated fatty acids include 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, 9-octadecenoic acid, 11-octadecenoic acid, 9-eicosenoic acid, cis-11-eicosenoic acid, cetoleic acid, 13-docosenoic acid, 15-tetracosenoic acid, 17-hexacosenoic acid, 6,9,12,15-hexadecatetraenoic acid, linoleic acid, linolenic acid, α-eleostearic acid, β-eleostearic acid, punicic acid, 6,9,12,15-octadecatetraenoic acid, parinaric acid, 5,8,11,14-eicosatetraenoic acid, 5,8,11,14,17-eicosapentaenoic acid, 7,10,13,16,19-docosapentaenoic acid, 4,7,10,13,16,19-docosahexaenoic acid, and the like.

Examples of suitable hydroxy fatty acids include α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, α-hydroxybehenic acid, 9-hydroxy-trans-10,12-octadecadienic acid, kamolenic acid, ipurolic acid, 9,10-dihydroxystearic acid, 12-hydroxystearic acid and the like. Examples of suitable polycarboxylic acids include oxalic acid, malonic acid, succinic acid, glutaric acid, adipic acid, pimelic acid, suberic acid, azelaic acid, sebacic acid, D,L-malic acid, and the like.

In some embodiments, each fatty acid is independently selected from Propionic acid, Butyric acid, Valeric acid, Caproic acid, Enanthic acid, Caprylic acid, Pelargonic acid, Capric acid, Undecylic acid, Lauric acid, Tridecylic acid, Myristic acid, Pentadecylic acid, Palmitic acid, Margaric acid, Stearic acid, Nonadecylic acid, arachidic acid, Heneicosylic acid, Behenic acid, Tricosylic acid, Lignoceric acid, Pentacosylic acid, Cerotic acid, Heptacosylic acid, Montanic acid, Nonacosylic acid, Melissic acid, Henatriacontylic acid, Lacceroic acid, Psyllic acid, geddic acid, ceroplastic acid, hexatriacontylic acid, heptatriacontanoic acid, or octatriacontanoic acid.

In some embodiments, each fatty acid is independently selected from α-linolenic acid, stearidonic acid, eicosapentaenoic acid, docosahexaenoic acid, linoleic acid, gamma-linoleic acid, dihomo-gamma-linoleic acid, arachidonic acid, docosatetraenoic acid, palmitoleic acid, vaccenic acid, paullinic acid, oleic acid, elaidic acid, gondoic acid, eurcic acid, nervonic acid, mead acid, adrenic acid, bosseopentaenoic acid, ozubondo acid, sardine acid, herring acid, docosahexaenoic acid, or tetracosanolpentaenoic acid, or another monounsaturated or polyunsaturated fatty acid.

In some embodiments, one or both of the fatty acids is an essential fatty acid. In view of the beneficial health effects of certain essential fatty acids, the therapeutic benefits of disclosed therapeutic-loaded exosomes may be increased by including such fatty acids in the therapeutic agent. In some embodiments, the essential fatty acid is an n-6 or n-3 essential fatty acid selected from the group consisting of linolenic acid, gamma-linolenic acid, dihomo-gamma-linolenic acid, arachidonic acid, adrenic acid, docosapentaenoic n-6 acid, alpha-linolenic acid, stearidonic acid, the 20:4n-3 acid, eicosapentaenoic acid, docosapentaenoic n-3 acid, or docosahexaenoic acid.

In some embodiments, each fatty acid is independently selected from all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid, or lipoic acid. In other embodiments, the fatty acid is selected from eicosapentaenoic acid, docosahexaenoic acid, or lipoic acid. Other examples of fatty acids include all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid). In some embodiments, the fatty acid is a medium-chain fatty acid such as lipoic acid.

Fatty acid chains differ greatly in the length of their chains and may be categorized according to chain length, e.g. as short to very long. Short-chain fatty acids (SCFA) are fatty acids with chains of about five or less carbons (e.g. butyric acid). In some embodiments, each of the fatty acids is independently a SCFA. In some embodiments, one of the fatty acids is independently a SCFA. Medium-chain fatty acids (MCFA) include fatty acids with chains of about 6-12 carbons, which can form medium-chain triglycerides. In some embodiments, each of the fatty acids is independently a MCFA. In some embodiments, one of the fatty acids is independently a MCFA. Long-chain fatty acids (LCFA) include fatty acids with chains of 13-21 carbons. In some embodiments, each of the fatty acids is independently a LCFA. In some embodiments, one of the fatty acids is independently a LCFA. Very long chain fatty acids (VLCFA) include fatty acids with chains of 22 or more carbons, such as 22-60, 22-50, or 22-40 carbons. In some embodiments, each of the fatty acids is independently a VLCFA. In some embodiments, one of the fatty acids is independently a VLCFA. In some embodiments, one of the fatty acids is independently a MCFA and one is independently a LCFA.

### III. Pharmaceutically Acceptable Compositions

According to another embodiment, the present disclosure provides a composition comprising a therapeutic-loaded milk vesicle of the present disclosure and a pharmaceutically acceptable carrier, adjuvant, or vehicle. The amount of therapeutic agent encapsulated or otherwise carried by a therapeutic-loaded milk vesicle is an amount effective to treat the relevant disease, disorder, or condition in a patient in need thereof. In certain embodiments, a composition as disclosed herein is formulated for administration to a patient in need of such composition. In some embodiments, a composition as disclosed herein is formulated for oral administration to a patient. The term “patient,” as used herein, means an animal, for example a mammal, such as a human.

The term “pharmaceutically acceptable carrier, adjuvant, or vehicle” refers to a non-toxic carrier, adjuvant, or vehicle that does not destroy the pharmacological activity of the therapeutic-loaded vesicle with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

In some embodiments, the therapeutic-loaded milk vesicles or pharmaceutical compositions thereof are administered by an oral, intravenous, subcutaneous, intranasal, inhalation, intramuscular, intraocular, intraperitoneal, intratracheal, transdermal, buccal, sublingual, rectal, topical, local injection, or surgical implantation route. In some embodiments, the administration route is oral.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

The pharmaceutical compositions for oral administration as described herein may be administered to a subject with or without food. In some embodiments, pharmaceutically acceptable compositions disclosed herein are administered without food. In other embodiments, pharmaceutically acceptable compositions of this invention are administered with food.

In some embodiments, the therapeutic, diagnostic, and prognostic attributes of therapeutic-loaded milk vesicles are achieved via non-oral means. Achieving systemic distribution of the encapsulated therapeutic agent using milk-derived vesicles following delivery would be the major objective of this approach but it is also possible to achieve selective delivery to sites of interest through the use of targeting ligands (e.g., antibodies, peptides, aptamers, or others: see, e.g., Friedman, A. D. et al., Curr Pharm Des 2013; 19(35): 6315-6329).

To aid in delivery of the therapeutic-loaded milk vesicles, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions.

Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of therapeutic-loaded milk vesicles of the present disclosure that may be combined with the carrier materials to produce a composition in a single dosage form will vary depending upon the host treated, the particular mode of administration, and other factors known to one of ordinary skill. Preferably, provided compositions should be formulated so that a dosage of between 0.01-100 mg/kg body weight/day of the therapeutic agent can be administered to a patient receiving these compositions.

It should also be understood that a specific dosage and treatment regimen for any particular patient will depend upon a variety of factors, including the activity of the specific therapeutic-loaded milk vesicle employed, the age, body weight, general health, sex, diet, time of administration, rate of excretion, drug combination, and the judgment of the treating physician and the severity of the particular disease being treated. The amount of a therapeutic-loaded milk vesicle of the present disclosure in the composition will also depend upon the particular therapeutic-loaded vesicle in the composition.

### IV. Use of Cargo-Loaded Milk Vesicles for Delivery of Cargo to Subjects and Treating Associated Diseases

In accordance with the present disclosure, a variety of biological molecules (cargos), including therapeutic agents and diagnostic agents, can be loaded or encapsulated inside a milk vesicle. Using a milk vesicle as a carrier enhances desirable properties of the biological molecule such as improving oral bioavailability, for example by minimizing destruction of the agent in the gut or minimizing liver first-pass effect; or improving therapeutic agent delivery to a target tissue; or increasing the solubility and stability of the therapeutic agents, including the solubility and stability of the agents in vivo. In one aspect, the therapeutic agent comprises or is chemically modified to comprise a hydrophobic group. Suitable hydrophobic groups include sterols, steroids, lipids, phospholipids, or synthetic or natural hydrophobic polymers. Without wishing to be bound by theory, it is believed that hydrophobic modification, e.g. lipid, sterol, or steroid tagging, of a therapeutic agent facilitates its loading into or onto milk vesicles, such that higher loading efficiencies are enabled.

To practice the methods described herein, an effective amount of any of the cargo-loaded milk vesicles can be administered to a subject in need of the treatment via a suitable route, e.g., those described herein. In one example, the cargo-loaded milk vesicle is administered orally. The cargo-loaded milk vesicle would be effective in treating or diagnosing target diseases of interest, depending upon the biological molecules loaded in the milk vesicle. In some embodiments, the disease, disorder, or condition is selected from a hyperproliferative disorder, viral or microbial infection, autoimmune disease, allergic condition, inflammatory disease, cardiovascular disease, metabolic disease, or neurodegenerative disease.

In some embodiments, the therapeutic agent can be used for diagnoses and prognosis of disease and measuring response to treatment. In another embodiment, following the administration of a therapeutic-loaded vesicle (for example, a therapeutic-loaded milk-derived vesicle), processing by or interaction with particular cell types yields markers that may be assessed through means known in the art to provide a diagnosis or prognosis or measure a response to treatment.

Any of the various therapeutic agents disclosed herein may be compatible with association and/or encapsulation in a milk vesicle according to the present disclosure. In some embodiments, the therapeutic agent is a biologic. In some embodiments, the biologic is selected from an iRNA, siRNA, miRNA, mRNA, ncRNA, or other oligonucleotide therapeutic.

The cargo-loaded milk vesicles as described herein is useful as a diagnostic, prognostic, or therapeutic in the context of cancer, autoimmune disorders, liver disorders, gene therapy, immuno-oncology, and other diseases, disorders, and conditions as described in detail herein. In another aspect, a therapeutic-loaded milk vesicle according to the present disclosure is useful in treating, preventing, or ameliorating a hyperproliferative disorder, viral or microbial infection, autoimmune disease, allergic condition, inflammatory disease, disorder, or condition, cardiovascular disease, metabolic disease, or neurodegenerative disease.

In some embodiments, the biological molecule in the cargo-loaded milk vesicles is an autoimmue antigen. Such cargo-loaded milk vesicle can be used to treat, prevent, or ameliorate an autoimmune disease, such as Rheumatoid Arthritis, Diabetes Mellitus, Insulin-DependentLupus Erythematosus (Systemic), Multiple Sclerosis, Psoriasis, Pancreatitis, Inflammatory Bowel Diseases, Crohn's disease, ulcerative colitis, Sjogren's Syndrome, autoimmune encephalomyelitis, experimental Graves' Disease, Sarcoidosis, Scleroderma, primary biliary cirrhosis, Chronic lymphocytic thyroiditis, Lymphopenia, Celiac Disease, Myocarditis, Chagas Disease, Myasthenia Gravis, Glomerulonephritis, IGA, Aplastic Anemia, Lupus Nephritis, Hamman-Rich syndrome, Hepatitis, Chronic Active Dermatomyositis, Glomerulonephritis, Membranous Mucocutaneous Lymph Node Syndrome, Pemphigoid, Bullous Behcet Syndrome, Spondylitis, Ankylosing Hepatitis, Autoimmune Cushing Syndrome, Guillain-Barre Syndrome, Cholangitis, Sclerosing Antiphospholipid Syndrome, Vitiligo, Thyrotoxicosis, Wegener's Granulomatosis, idiopathic purpura, Raynaud's Thrombocytopenia, Autoimmune hemolytic anemia, Cryoglobulinemia, Mixed Connective Tissue Disease, Temporal Arteritis, Pemphigus Vulgaris, Addison's Disease, Rheumatic Fever, pernicious anemia, Alopecia Areata, Lupus Erythematosus, Discoid Narcolepsy, Takayasu's Arteritis, autoimmune neuritis, Experimental Polyarteritis Nodosa, Polymyalgia Rheumatica, Dermatitis Herpetiformis, Autoimmune Myocarditis, Meniere's Disease, Chronic Inflammatory Demyelinating Polyneuropathy, Lambert-Eaton Myasthenic Syndrome, Lichen Sclerosus et Atrophicus, Churg-Strauss Syndrome, Erythematosis, Reiter Disease, Anti-Glomerular Basement Membrane Disease, autoimmune polyendocrinopathies, Felty's Syndrome, Goodpasture Syndrome, Achlorhydria, Autoimmune Lymphoproliferative Polyradiculoneuropathy, Uveomeningoencephalitic Syndrome, Polychondritis, Relapsing Atopic Allergy, Idiopathic thrombocytopenia, Stiff-Person Syndrome, Autoimmune Polyendocrinopathy-Candidiasis-Ectodermal-Dystrophy, Epidermolysis, Bullosa Acquisita, Autoimmune orchitis, Oculovestibuloauditory syndrome, Ophthalmia, Sympathetic Myelitis, Transverse Diffuse Cerebral Sclerosis of Schilder, Neuromyelitis Optica, Still's Disease, Adult Onset Autoimmune oophoritis, Mooren's ulcer, Autoimmune Syndrome Type II, Polyglandular Autoimmune hypophysitis, Lens-induced uveitis, pemphigus foliaceus, Opsoclonus-Myoclonus Syndrome, Type B Insulin Resistance, Autoimmune Atrophic Gastritis, Lupus Hepatitis, Autoimmune Hearing Loss, Acute hemorrhagic leukencephalitis, autoimmune hypoparathyroidism, or Hashimoto's Thyroidosis.

Additional examples include Addison's disease, Agammaglobulinemia, Alopecia areata, Amyloidosis, Ankylosing spondylitis, Anti-GBM/Anti-TBM nephritis, Antiphospholipid syndrome (APS), Autoimmune hepatitis, Autoimmune inner ear disease (AIED), Axonal & neuronal neuropathy (AMAN), Behcet's disease, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Cicatricial pemphigoid/benign mucosal pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, IgA Nephropathy, IgG4-related sclerosing disease, Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, chronic Lyme disease, Meniere's disease, Microscopic polyangiitis (MPA), Mixed connective tissue disease (MCTD), Mooren's ulcer, Mucha-Habermann disease, Multiple sclerosis (MS), Myasthenia gravis, Myositis, Narcolepsy, Neuromyelitis optica, Neutropenia, Ocular cicatricial pemphigoid, Optic neuritis, Palindromic rheumatism (PR), PANDAS (Pediatric Autoimmune Neuropsychiatric Disorders Associated with Streptococcus), Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome (polyneuropathy, organomegaly, endocrinopathy, monoclonal gammopathy, skin changes), Polyarteritis nodosa, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Reiter's syndrome, Relapsing polychondritis, Restless legs syndrome (RLS), Retroperitoneal fibrosis, Rheumatic fever, Rheumatoid arthritis (RA), Sarcoidosis, Schmidt syndrome, Scleritis, Scleroderma, Sjogren's syndrome, Sperm and testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Wegener's granulomatosis (now termed Granulomatosis with Polyangiitis (GPA).

In some embodiments, the present disclosure provides a method of modulating an immune response, comprising administering to a patient in need thereof an effective amount of a therapeutic-loaded milk vesicle. In some embodiments, the patient is suffering from a hyperproliferative disease, disorder, or condition such as cancer. In some embodiments, the patient is suffering from an autoimmune disease, disorder, or condition. In some embodiments, the therapeutic agent's target in vivo is one of those listed in Table 6, below. In some embodiments, the therapeutic-loaded milk vesicle is administered in combination with a compound listed in Table 6, or a pharmaceutically acceptable salt thereof. In some embodiments, the therapeutic agent loaded in the vesicle and the coadministered compound of Table 6 modulate a target in Table 6. Abbreviations used in Table 6 are shown below:

AMPCP, adenosine 5′-(α,β methylene)diphosphate; ARG, arginase; COX2, cyclooxygenase 2; CSF, colony stimulating factor; CTL, cytotoxic T lymphocyte; DC, dendritic cell; HIF1α, hypoxia-inducible factor 1α; IDO, indoleamine 2,3-dioxygenase; IFN, interferon; IL, interleukin; iNOS, inducible nitric oxide synthase; MDSC, myeloid-derived suppressor cell; MOA, mechanism of action; MSP, macrophage-stimulating protein; NK, natural killer; PDE5, phosphodiesterase type 5; PGE₂, prostaglandin E2; PMNC, peripheral mononuclear cell; ROS, reactive oxygen species; TAF, tumour-associated fibroblasts; TAM, tumour-associated macrophage; TCR, T cell receptor; TDO, tryptophan 2,3-dioxygenase; T helper; TGFβ, transforming growth factor-β; TLR, Toll-like receptor; TME, tumor microenvironment; TNF, tumour necrosis factor; T_{Reg}, regulatory T; TSP1, thrombospondin 1.
Listed are small-molecule drug targets that have been proposed for cancer immunotherapy.
For some examples, the clinical development status provided is for a non-immuno oncology indication. In these cases the literature supports clinical consideration in light of its impact on innate immune function. While the scientific literature illustrates CXCR2 antagonism using a mAb, several small-molecule CXCR1 and CXCR2 antagonists have reached clinical trials and in principle could show similar efficacy.

Other utilities of the cargo-loaded mile vesicles can be found in WO2018102397 and references cited therein, the relevant disclosures of each of which are incorporated by reference for the purposes or subject matter referenced herein.

TABLE 6
Immuno-oncology Targets

| | | | | | | |
|---|---|---|---|---|---|---|
| | | | Compound | Company or | | |
| Target | Location | Function | (MOA) | institution | Model or indication | Status [symbol] |

Amino acid catabolism

| | | | | | | |
|---|---|---|---|---|---|---|
| IDO | Macrophages, | Depletion of tryptophan | INCB24360 | Incyte | Murine syngeneic | Phase II |
| | DCs, upregulated | and metabolites promote | (inhibitor) | | tumour (PAN02) | |
| | in tumours | T_{Reg} cell differentiation, | 1-Methyl | NewLink Genetics | Murine syngeneic | Phase I |
| | | suppression of immune | tryptophan | | tumour model (Lewis | |
| | | response and decreased | (inhibitor) | | lung cancer) | |
| | | DC function | NLG919 | NewLink Genetics | Murine syngeneic | Phase I |
| | | | (inhibitor) | | tumour (PAN02) | |
| TDO | Hepatocytes | Depletion of tryptophan | LM10 (inhibitor) | Ludwig institute for | Murine syngeneic | Research |
| | | and metabolites promote | | Cancer Research | tumour (P815B/TDO) | |
| | | T_{Reg} cell differentiation, | | | | |
| | | suppression of immune | | | | |
| | | response and decreased | | | | |
| | | DC function | | | | |
| ARG1, | MDSCs, TAMs, | Depletion of the CD3ζ | Compound 9 | The Institutes for | Reperfusion injury | Research |
| ARG2 | vascular | chain of the TCR | (inhibitor) | Pharmaceutical | from myocardial | |
| | endothelium | suppresses T cell | | Discovery | ischaemia | |
| | | responses to antigen | | | | |
| iNOS, | MDSCs | Supports generation of | NCX-401 (dual | NicOx | Preventing colorectal | Phase II, |
| ARG1, | | ROS that modify CCL2 | inhibitor) | | carcinoma | discontinued |
| ARG2 | | levels, disabling T cell | AT38 (dual | Istituti di Ricovero | MCA-203 fibrosar- | Research |
| | | chemotaxis | inhibitor) | e Cura a Carattere | coma-bearing mice | |
| | | | | Scientifico (IRCCS) | | |
| PDE5 | MDSCs | Decreases functional IL-13 | Tadalafil | Eli Lilly and | Investigational for | Approved |
| | | receptors | (inhibitor) | Company | immuno-oncology | for erectile |
| | | | | | | dysfunction |
| | | | | | | and |
| | | | | | | hypertension |

Signalling of tumour-derived extracellular ATP

| | | | | | | |
|---|---|---|---|---|---|---|
| PZX7 | Broadly | Induction of IL-1β | ATP (agonist) | Istituti di Ricovero | Immuno-stimulant | Research |
| | expressed on | release in DCs, enhances | | e Cura a Carattere | | |
| | lymphocytes, | tumour-specific CD8 T cell | | Scientifico (IRCCS) | | |
| | often upregulated | cytotoxicity | | | | |
| | in tumours | | | | | |
| | Broadly | Increases CCL2, ROS, | AZ10606120 | University of | Murine B16F10 | Research |
| | expressed on | ARG1 and TGFβ levels; | (antagonist) | Ferrara, ltaly | melanoma | |
| | lymphocytes, | activates MDSCs, | | | | |
| | often upregulated | tumour growth and | | | | |
| | in tumours | angiogenesis | | | | |
| P2Y [symbol] | ATP derived from | Inhibits synthesis of IL-1, | NF340 | University of | Immuno-stimulant | Research |
| | tumour binds | TNFα, IL-6; increases | (antagonist) | Duesseldorf, | | |
| | receptor on DCs | secretion of TSP1, IL-10 | | Germany | | |
| | | and IDO1, resulting in DC | | | | |
| | | semi-maturation | | | | |

Adenosine signalling

| | | | | | | |
|---|---|---|---|---|---|---|
| A [symbol] | T_{Reg} cells, DCs, NK | Elevated cAMP | SCH58261 | Peter MacCallum | B16 melanoma | Research |
| receptor | cells, NK T cells, | blunts TCR-mediated | (antagonist) | Cancer Centre, | metastasis | |
| | tumours | cytotoxicity: inhibits | | Victoria, Australia | | |
| | | effector T cells; expands | | | | |
| | | T_{Reg} cells: enhances NK cell | | | | |
| | | cytotoxicity | | | | |
| | T_{Reg} cells, DCs, NK | Elevated cAMP | SCH420814 | Merck | Parkinson disease | Phase III, |
| | cells, NK T cells, | blunts TCR-mediated | (antagonist) | | | discontinued |
| | tumours | cytotoxicity: inhibits | | | | |
| | | effector T cells; expands | | | | |
| | | T_{Reg} cells: enhances NK cell | | | | |
| | | cytotoxicity | | | | |
| A [symbol] | Myeloid cells, | Elevated cAMP increases | PSB1115 | University of | Murine B16 F10 | Research |
| receptor | expression driven | IL-10 and CCL2 levels: | (antagonist) | Salerno, Italy | melanoma | |
| | by HIF1α | expansion of MDSCs | | | | |
| | | and TAMs | | | | |

Adenosine production

| | | | | | | |
|---|---|---|---|---|---|---|
| CD39 | T_{Reg} cells, B cells, | Contributes to the | ARL 67176 | OREGA Biotech | Murine B16 F10 | Research |
| | MDSCs, NK | production of adenosine, | (inhibitor) | | melanoma | |
| | cells, tumours, | which binds to A [symbol] , A [symbol] , A [symbol] | | | | |
| | endothelium | and A [symbol] , receptors | | | | |
| CD73 | T_{Reg} cells, B cells, | Contributes to the | AMPCP | Cancer Therapy | Murine B16 F10 | Research |
| | MDSCs, NK | production of adenosine, | (inhibitor) | and Research | melanoma | |
| | cells, tumours, | which binds to A [symbol] , A [symbol] , A [symbol] | | Center, University | | |
| | endothelium | and A [symbol] , receptors | | of Texas San | | |
| | | | | Antonio, USA | | |

Elevation of cyclic AMP

| | | | | | | |
|---|---|---|---|---|---|---|
| COX2 | MDSCs, TAMs, | Generates PGE [symbol] , which is | Celecoxib | Pfizer | Rheumatoid arthritis, | Approved |
| | T_{Reg} cells, tumours | immunosuppressive (via | (inhibitor) | | osteoarthritis, pain | |
| | | EP [symbol] and EP [symbol] receptors) | | | | |
| EP [symbol] | MDSCs, NK cells, | T_{Reg} cell activation: | PF-04418948 | Pfizer | None indicated | Phase I, |
| receptor | T_{Reg} cells, tumours | tumour proliferation | (antagonist) | | | discontinued |
| | | and angiogenesis | | | | |
| EP [symbol] | MDSCs, NK cells, | Activates suppressor | RQ-15986 | RaQualia Pharma | Murine mammary 66.1 | Preclinical |
| receptor | T_{Reg} cells, tumours | cell function of MDSCs | (antagonist) | | tumour metastasis | |
| | | and TAMs | | | | |

Chemokines and chemokine receptors

| | | | | | | |
|---|---|---|---|---|---|---|
| CXCR1, | PMNCs, | Migration of CXCR2 | CXCR2-specifi | Pediatric Oncology | Murine | Research |
| CXCR2 | monocytes, | expressing MDSCs into | mAb [symbol] | Branch, National | rhabdomyosarcoma | |
| | endothelium, | the TME; directs effects on | (antagonist) | Cancer Institute, | | |
| | mast cells | tumour proliferation | | National Institutes | | |
| | | | | of Health, USA | | |
| CXCR4 | T cells, B cells, | Ligand expression | Plerixafor | Sanofi-Aventis, | Pancreactic ductal | Approved |
| | monocytes, | in stroma mediates | (also known | Cancer | adenocarcinoma | for stem cell |
| | PMNCs, immature | metastasis by | as AMD3100) | Research UK | | mobilization |
| | DCs, tumours | tumour-specific and | (antagonist) | | | |
| | | T cell-based mechanisms | | | | |
| CCR2 | Monocytes, | Drives TAM and monocytic | PF-4136109 | Pfizer, Washington | Murine pancreatic | Phase IB |
| | PMNCs, immature | MDSC infiltration into | (antagonist) | University School | model supportive | |
| | DCs, T cells, | the TME | | of Medicine, | of clinical study | |
| | NK cells | | | National Cancer | | |
| | | | | Institute, USA | | |
| CCR5 | T [symbol] 1 cells, | T_{Reg} cell infiltration and | Maraviroc | National Center for | Blockade of | Phase I |
| | CD8 [symbol] T cells, | infiltration of precursors to | (antagonist) | Tumour Diseases, | metastatic | |
| | monocytes, | generate TAMs and MDSCs | | Germany | colorectal cancer | |
| | macrophages | | | | | |

Recognition of foreign organisms to activate the immune response

| | | | | | | |
|---|---|---|---|---|---|---|
| TLR4 | Monocytes, | Bacterial host defence; | OM-174 | Centre Hospitalier | Rat colon cancer, | Phase I |
| | macrophages, | activation results in | (agonist) | Universitaire, | solid tumours | |
| | DCs | cytokine burst (IL-1, | | France | | |
| | | TNFα and type [symbol] IFNs) | | | | |
| TLR7, | DCs, | Binds to viral ssRNA and | Imiquimod | Graceway | Basal cell carcinoma | Approved |
| TLR8 | plasmacytoid | bacterial DNA; induces | (agonist) | Pharmaceuticals | | |
| | DCs, | secretion of inflammatory | | | | |
| | macrophages | cytokines and type 1 | | | | |
| | | IFN, which promotes a | | | | |
| | | T [symbol] 1-directed activation | | | | |
| | | of DCs and NK cells to | | | | |
| | | directly kill tumour cells | | | | |
| | | and suppress T_{Reg} cells | | | | |
| TLR7 | DCs, | Host defence recognizing | 852A(agonist) | Pfizer | Solid and | Phase I/II |
| | plasmacytoid DCs, | viral ssRNA and bacterial | | | haematological | |
| | macrophages | DNA; inflammatory | | | malignancies | |
| | | cytokines and type [symbol] IFN | | | | |
| | | secretion promoting a | | | | |
| | | T [symbol] 1-directed activation | | | | |
| | | of DCs and NK cells to | | | | |
| | | directly kill tumour cells | | | | |
| | | and suppress T_{Reg} cells | | | | |
| TLR8 | DCs, | Host defence recognizing | VTX-2337 | VentiRx | Solid and | Phase I/II |
| | plasmacytoid DCs, | viral ssRNA and bacterial | (agonist) | Pharmaceuticals | haematological | |
| | macrophages | DNA; inflammatory | | | malignancies | |
| | | cytokines and type [symbol] IFN | | | | |
| | | secretion promoting a | | | | |
| | | T [symbol] 1-directed activation | | | | |
| | | of DCs and NK cells to | | | | |
| | | directly kill tumour cells | | | | |
| | | and suppress T_{Reg} cells | | | | |
| TLR9 | DCs, | Host defence recognizing | IMO-2055 | Hybridon, Idera | Advanced solid | Phase I/II |
| | plasmacytoid DCs, | viral ssRNA and bacterial | (agonist) | Pharmaceuticals | malignancies | |
| | macrophages | DNA; inflammatory | | | | |
| | | cytokines and type [symbol] IFN | | | | |
| | | secretion promoting a | | | | |
| | | T [symbol] 1-directed activation | | | | |
| | | of DCs and NK cells to | | | | |
| | | directly kill tumour cells | | | | |
| | | and suppress T_{Reg} cells | | | | |

Signal transduction: kinase inhibitors

| | | | | | | |
|---|---|---|---|---|---|---|
| ALK5 | Downstream | Attenuation of TGFβ | LY2157299 | Eli Lilly and | Murine B16 F10 | Phase I/II |
| | of TGFβ, | signalling causes activation | | Company | melanoma | |
| | which is often | of CD8 [symbol] cells, generation of | EW-7197 | Ewha Womens | Murine B16 F10 | Phase I |
| | overexpressed | CTLs, and stimulation of | | University, Seoul, | melanoma | |
| | by tumours | NK cells | | Korea | | |
| BRAF [symbol] | Tumours | V600E-driven IL-1 | Vemurafenib | Plexxikon, | Patients with | Approved |
| | | expression promotes | Dabrafenib | Genentech, | melanoma | for |
| | | immunosuppressive TAF | | GlaxoSmithKline, | | metastatic |
| | | and MDSC function | | MD Anderson | | melanoma |
| | | | | Cancer Center, USA | | |
| RON | Expressed on | Decreases IL-12, IFNγ and | BMS-777607 | Bristol-Myers | Inhibits metathesis | Phase I/II |
| | myeloid cells. | TNF, and increases IL-10; | | Squibb, Huntsman | in MMTV-PyMT | |
| | Tumours secrete | favours M2 phenotype | | Cancer institute, | transgenic mice | |
| | its ligand MSP | | | Utah, USA | | |
| CSF1 | Glioma cells and | M1 to M2 polarization, | BLZ945 | Memorial | Murine glioblastoma | Research |
| | TAMs express CSF | which promotes tumour | | Sloan-Kettering | | |
| | ligand | growth and survival | | Cancer Center, | | |
| | | | | New York, USA | | |
| PI3Kδ | B cells, T cells, | Inhibition preferentially | PI-3065 | Piramed Pharma, | 4T1 breast cancer and | Research |
| | myeloid lineage | suppresses T_{Reg} cell | | University CoIlege | other solid tumours | |
| | cells | function, resulting in | | London Cancer | | |
| | | effector T cell activation | | Institute, UK | | |
| PI3Kγ | Haematopoietic | Required for | TG100-115 | University of | Lewis lung | Research |
| | cells, primarily | α4β1-dependent | | California San | carcinoma and PyMT | |
| | myeloid lineage | myeloid coll infiltration | | Diego, Moores | spontaneous | |
| | | into tumours | | Cancer Center, USA | breast carcinomas | |

[symbol]

indicates data missing or illegible when filed

### V. Combination Therapies

Any of the cargo-loaded milk vesicles described herein or pharmaceutically acceptable composition thereof, may be administered to a patient in need thereof in combination with one or more additional therapeutic agents and/or therapeutic processes.

A cargo-loaded milk vesicle of the present disclosure can be administered alone or in combination with one or more other therapeutic compounds, possible combination therapy taking the form of fixed combinations or the administration of a therapeutic-loaded milk vesicle of the disclosure and one or more other therapeutic compounds being staggered or given independently of one another, or the combined administration of fixed combinations and one or more other therapeutic compounds. A therapeutic-loaded milk vesicle of the present disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Such additional agents may be administered separately from a provided therapeutic-loaded milk vesicle-containing composition, as part of a multiple dosage regimen. Alternatively, those agents may be part of a single dosage form, mixed together with a therapeutic-loaded milk vesicle of the present disclosure in a single composition. If administered as part of a multiple dosage regime, the two active agents may be submitted simultaneously, sequentially or within a period of time from one another.

As used herein, the term “combination,” “combined,” and related terms refers to the simultaneous or sequential administration of therapeutic agents in accordance with this disclosure. For example, a therapeutic-loaded milk vesicle of the present disclosure may be administered with another therapeutic agent simultaneously or sequentially in separate unit dosage forms or together in a single unit dosage form. Accordingly, the present invention provides a single unit dosage form comprising a therapeutic-loaded milk vesicle of the present disclosure, an additional therapeutic agent, and a pharmaceutically acceptable carrier, adjuvant, or vehicle. In some embodiments, the additional agent is encapsulated in the same milk vesicle as the first therapeutic agent. In some embodiments, the additional agent is encapsulated in a different milk vesicle than the first therapeutic agent. In some embodiments, the additional agent is not encapsulated in an milk vesicle. In some embodiments, the additional agent is formulated in a separate composition from the therapeutic-loaded milk vesicle.

The amount of both a disclosed therapeutic-loaded milk vesicle and additional therapeutic agent (in those compositions which comprise an additional therapeutic agent as described above) that may be combined with the carrier materials to produce a single dosage form will vary depending upon the patient treated and the particular mode of administration. In certain embodiments, compositions of this disclosure should be formulated so that a dosage of between 0.01-100 mg/kg body weight/day of a disclosed therapeutic-loaded milk vesicles can be administered.

In those compositions which comprise an additional therapeutic agent, that additional therapeutic agent and the therapeutic-loaded milk vesicle of the present disclosure may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Examples of agents with which the therapeutic-loaded milk vesicle of the present disclosure may be combined include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, include a monoclonal antibody or a siRNA therapeutic, which may or may not be encapsulated in a disclosed milk vesicle.

In another embodiment, the present invention provides a method of treating an inflammatory disease, disorder or condition by administering to a patient in need thereof a cargo-loaded milk vesicle and one or more additional therapeutic agents. Such additional therapeutic agents may be small molecules or a biologic and include, for example, acetaminophen, non-steroidal anti-inflammatory drugs (NSAIDS) such as aspirin, ibuprofen, naproxen, etodolac, and celecoxib, colchicine, corticosteroids such as prednisone, prednisolone, methylprednisolone, hydrocortisone, and the like, probenecid, allopurinol, febuxostat, and sulfasalazine. Other examples include monoclonal antibodies such as tanezumab, anticoagulants such as heparin and warfarin, antidiarrheals such as diphenoxylate, and loperamide, bile acid binding agents such as cholestyramine, alosetron, and lubiprostone, anticholinergics or antispasmodics such as dicyclomine, beta-2 agonists such as albuterol and levalbuterol, anticholinergic agents such as ipratropium bromide and tiotropium, inhaled corticosteroids such as beclomethasone dipropionate and triamcinolone acetonide.

A therapeutic-loaded exosome of the current invention may also be used to advantage in combination with an antiproliferative compound. Such antiproliferative compounds include, but are not limited to, aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

Additional therapeutic agents for co-use with the cargo-loaded milk vesicles as described herein are known in the art and/or disclosed in WP2018102397 and the references cited therein, the relevant disclosures of each of which are incorporated by reference for the purposes or subject matter referenced herein.

### VI. Methods of Making Milk Vesicles and Loading with Cargos

In one aspect, a milk vesicle may be harvested from primary sources of a milk-producing animal. In some embodiments, the milk vesicle is derived (e.g., isolated or manipulated) from milk or colostrum or milk component from any of a suitable mammal source. Examples include a cow, human, buffalo, goat, sheep, camel, donkey, horse, reindeer, moose, or yak. In some embodiments, the milk is from a cow. In some embodiments, the milk or colostrum is in powder form. In some embodiments, the milk vesicles are produced and subsequently isolated from mammary epithelial cells lines adapted to recapitulate the milk vesicle architecture of that naturally occurring in milk. In another aspect, suitable milk vesicles are isolated from milk produced by a transgenic cow or other milk-producing mammal whose characteristics are optimized for producing milk vesicles with desirable properties for drug delivery, e.g., oral drug delivery.

In one aspect, the milk vesicles are provided using a cell line one in a batch-like process, wherein the milk vesicles may be harvested periodically from the cell line media. The challenge with cell line based production methods is the potential for contamination from exosomes present in fetal bovine serum (media used to grow cells). In another aspect, this challenge can be overcome with the use of suitable serum free media conditions so that milk vesicles purified from the cell line of interest are harvested from the culture medium.

In one aspect, the milk vesicles are isolated or derived from a milk or colostrum solution. Separation of milk vesicles from the bulk solution must be performed with care. In some embodiments, a filter such as a 0.2 micron filter is used to remove larger debris from solution. In some embodiments, the method for separation of milk milk vesicle (for example, in the 80-120 nanometer range) includes separation based on specific milk vesicle properties such as size, charge, density, morphology, protein content, lipid content, or epitopes recognized by antibodies on an immobilized surface (immuno-isolation).

In some embodiments, the separation method comprises a centrifugation step. In some embodiments, the separation method comprises PEG based volume excluding polymers.

In some embodiments, the separation method comprises ultra-centrifugation to separate the desired milk vesicles from bulk solution. In some embodiments, sequential steps involving initial spins at 20,000×g for up to 30 minutes followed by multiple spins at ranges of about 100,000×g to about 120,000×g for about 1 to about 2 hours provides a pellet or isolate rich in milk-derived vesicles.

In some embodiments, ultracentrifugation provides milk-derived vesicles that can be resuspended, for example, in phosphate buffered saline or a solution of choice. In some embodiments, the vesicles are further assessed for desired properties by assessing their attributes when exposed to a sucrose density gradient and picking the fraction in 1.13-1.19 g/mL range.

In other embodiments, isolation of vesicles of the present disclosure includes using combinations of filters that exclude different sizes of particles, for example 0.45 μM or 0.22 μM filters can be used to eliminate vesicles or particles bigger than those of interest. Milk vesicles may be purified by several means, including antibodies, lectins, or other molecules that specifically bind vesicles of interest, eventually in combination with beads (e.g. agarose/sepharose beads, magnetic beads, or other beads that facilitate purification) to enrich for the desired vesicles. A marker derived from the vesicle type of interest may also be used for purifying vesicles. For example, vesicles expressing a given biomarker such as a surface-bound protein may be purified from cell-free fluids to distinguish the desired vesicle from other types. Other techniques to purify vesicles include density gradient centrifugation (e.g. sucrose or optiprep gradients), and electric charge separation. All these enrichment and purification techniques may be combined with other methods or used by themselves. A further way to purify vesicles is by selective precipitation using commercially available reagents such as ExoQuick™ (System Biosciences, Inc.) or Total Exosome Isolation kit (Invitrogen™ Life Technologies Corporation).

Suitable milk vesicles may also be derived by artificial production means, such as from exosome-secreting cells and/or engineered as is known in the art.

In some embodiments, milk vesicles can be further characterized by one or more of nanoparticle tracking analysis to assess particle size, transmission electron microscopy to assess size and architecture, immunogold labeling of vesicles or their contents prior to electron microscopy to track species of interest associated with exosomes, immunoblotting, or protein content assessment using the Bradford Assay.

Various methods are known in the art to encapsulate a therapeutic agent in a vesicle that is compatible with the present disclosure. Accordingly, the present disclosure provides a method of encapsulating or loading a disclosed therapeutic agent in a milk-derived vesicle. In some embodiments, the method comprises the step of exposing the vesicle to electroporation, sonication, saponification, extrusion, freeze/thaw cycles, or partitioning of the therapeutic agent and the vesicle in a mixture of two or more solvents, to effect encapsulation or loading of the therapeutic agent in the vesicle.

In some embodiments, isolation of the milk vesicle is achieved by centrifuging raw (i.e., unpasteurized and/or unhomogenized milk or colostrum) at high speeds to isolate the vesicle. In some embodiments, a milk-derived vesicle is isolated in a manner that provides amounts greater than about 50 mg (e.g., greater than about 300 mg) of vesicles per 100 mL of milk. In some embodiments, the present invention provides a method of isolating a milk-derived vesicle comprising the steps of: providing a quantity of milk (e.g., raw milk or colostrum); and performing a centrifugation, e.g. sequential centrifugations, on the milk to yield greater than about 50 mg of milk-dervied vesicles per 100 mL of milk. In some embodiments, the sequential centrifugations yield greater than 300 mg of milk-derived vesicles per 100 mL of milk. In some embodiments, the series of sequential centrifugations comprises a first centrifugation at 20,000×g at 4° C. for 30 min, a second centrifugation at 100,000×g at 4° C. for 60 min, and a third centrifugation at 120,000×g at 4° C. for 90 min. In some embodiments, the isolated vesicles can then be stored at a concentration of about 5 mg/mL to about 10 mg/mL to prevent coagulation and allow the isolated vesicles to effectively be used for the encapsulation or loading of one or more therapeutic agents. In some embodiments, the isolated vesicles are passed through a 0.22 μm filter to remove any coagulated particles as well as microorganisms, such as bacteria.

In some embodiments, provided here are methods for isolating milk vesicles (e.g., those disclosed herein), wherein the methods involve one or more steps to reduce or eliminate caseins and/or lactoglobulins from the input milk materials. Caseins are the majority of proteins in milk that have a molecular weight or about 25-30 kDa. Lactoglobulins are the majority of proteins in milk that have a molecular weight of about 10-20 kDa. Briefly, such a method may involve one or more defatting steps to remove abundanct milk proteins and/or fats to produce defatted milk samples following conventional methods or those disclosed herein. The defactted milk samples can then be subject to one or more steps to disrupt casein micelles, coagulate casein and remove casein from the milk sample. The casein-depleted milk sample can thus be subject to steps to enrich milk vesicles, for example, those approached known in the art or disclosed herein, e.g., chromatography-based methods (e.g., for scalable preparation) and ultracentrifugation-based methods.

Any approaches known in the art for removing caseins can be used in the methods disclosed herein. In some embodiments, casein removal may be achieved chemically, e.g., by acidification. For example, a suitable acid solution (e.g., acetic acid, hydrochloric acid, citric acid, etc.) or powder of a suitable acid (e.g., citric acid powder) can be added into a milk sample such as a defatted milk sample to cause coagulation of casein or casein micelles, which can be removed by a conventional method, e.g., low-speed centrifugation (e.g., ≤20,000 g) or filtration. Alternatively, acidification of milk may be achieved by saturation of the milk with CO₂ gas.

In other embodiments, casein removal may be achieved using enzymes capable of coagulating or digesting casein, for example, using rennet. As used herein, “rennet” refers to a mixture of enzymes capable of curdling caseins in milk. In some examples, the rennet used in the methods disclosed herein is derived from an animal, e.g., a complex set of enzymes produced in the stomachs of a ruminant mammal such as calf. Such a rennet may comprise chymosin, which is a protease enzyme that curdles casein in milk, and optionally other enzymes such as pepsin and lipase. In other examples, the rennet used in the methods disclosed herein is derived from a plant, e.g., a vegetable rennet. Vegetable rennet can be an enzyme or a mixture of enzymes that coagulates milk and separates the curds and whey from milk. In some instances, the vegetable rennet used herein can be a commercially available vegetable rennet extracted from a mold such as Mucor miehei. Alternatively, one or more recombinant casein coagulation enzymes may be used for casein removal. Such recombinant enzymes may be produced using a suitable host (e.g., bacterium, yeast, insect cell, or mammalian cell) by the conventional recombinant technology.

In yet other embodiments, the method disclosed herein may involve the use of a Ca²⁺ chelating agent such as EDTA or EGTA to disrupt casein micelles, which can be then removed.

After removal of caseins (partially or completely), the milk sample can be subject to one or more steps to enrich the milk vesicles contained therein, e.g., ultracentrifugation, size exclusion chromatography, affinity purification, tangential flow filtration, or a combination thereof. In some examples, the method disclosed herein may comprise a tangential flow filtration (TFF) step for milk vesicle enrichment. In some instances, the method may further comprise a size exclusion chromatography following the TFF step. Alternatively, the enrichment may be achieved by a conventional approach such as ultracentrifugation.

In some embodiments, a milk vesicle composition described herein further includes one or more microRNAs (miRNAs) loaded into the vesicle, either by virtue of being present in the vesicles upon their isolation or by virtue of loading a miRNA for use as a therapeutic agent into the vesicles subsequent to their initial isolation. In some embodiments, the miRNA loaded into the vesicle is not naturally occurring in the source of the vesicles. For example, mammalian milk vesicles sometimes include loaded miRNAs in their natural state, and such miRNAs remain loaded in the vesicles upon their isolation. Such naturally-occurring miRNAs are distinguished from any miRNA therapeutic agent (or other iRNA, oligonucleotide, or other biologic) that is artificially loaded into the vesicles.

Loading into the vesicles, e.g., encapsulation, can be verified by disrupting the membrane of the therapeutic-loaded milk-derived vesicles, e.g., with a detergent to release its contents. The contents level can be evaluated, for example, via protein/RNA/DNA quantification assays.

In some embodiments, the presently disclosed milk-derived vesicles are able to deliver their cargo while withstanding stressed conditions or conditions under which the therapeutic agent would become deactivated, metabolized, or decomposed, e.g. saliva, digestive enzymes, acidic conditions in the stomach, peristaltic motions, and/or exposure to the various proteases, lipases, amylases, and nucleases that break down ingested components in the gastrointestinal tract.

### VII. Definitions

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

The terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the properties sought to be obtained within the scope of the present invention.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, ranges can be expressed as from “about” one particular value, or “about” one value to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if the range of “10-15” is disclosed, then 11, 12, 13, and 14 are also disclosed.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence

As used herein, the term “cancer” refers to all types of cancer or neoplasm or malignant tumors found in animals, including leukemias, carcinomas, melanoma, and sarcomas.

By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some embodiments, the cancer is selected from the group consisting of breast cancer, uterine cancer, lung cancer, prostate cancer, ovarian cancer, cervical cancer, and pancreatic cancer.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Isolation of Exosomes from Milk

Exosomes were isolated from raw milk (bovine and goat) using either size exclusion chromatography methods or differential ultracentrifugation. Both procedures are detailed below.

### Isolation Using Ultracentrifugation

Excess fats were removed from raw milk prior to purification by size exclusion chromatography. Milk was first centrifuged at 13,000 RCF for 30 minutes at 4° C. in a Optima XE-90 ultracentrifuge. After completion of centrifugation step, the supernatant was decanted away from the pellet and subsequently filtered to remove any remaining solids. Filtered supernatant was mixed with phosphate-buffered saline (PBS) and centrifuged for a second time at 100,000 RCF for 69 minutes at 4° C. in an Optima XE-90 ultracentrifuge. Supernatant is removed from the pellet and centrifuged for a third time, at 135,000 RCF for 103 minutes at 4° C. in an Optima XE-90 ultracentrifuge.

Supernatant was then carefully removed by pipetting the supernatant away from the pellet. The pellet (which contains the exosomes) was subsequently resuspended in PBS and centrifuged at 135,000 RCF for 103 minutes at 4° C. in a Optima XE-90 ultracentrifuge. The pellet is resuspended in PBS and centrifuged at 135,000 RCF for 103 minutes at 4° C. for two additional cycles to completely wash the pelleted exosomes. After the third washing (and fourth centrifugation at 135,000 RCF), the exosomes are resuspended in PBS and are available for further experimental use.

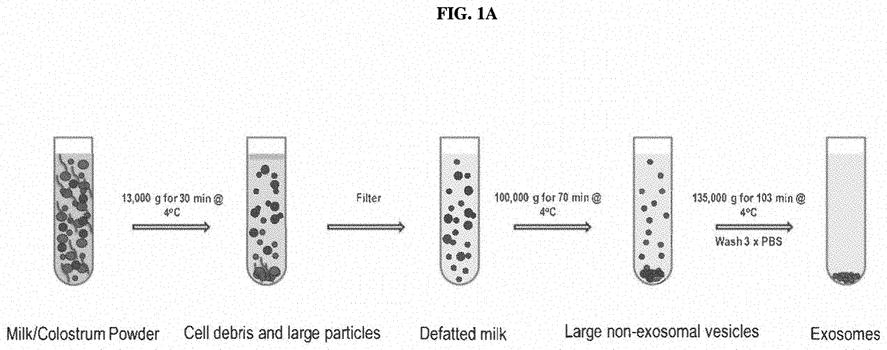

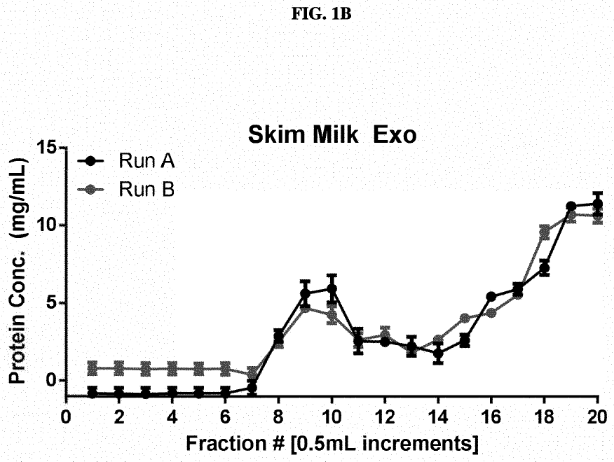

An exemplary flowchart for using ultracentrifugation to isolate milk exosome is provided in FIG. 1A. Results obtained from this procedure are probived in Table 7 below.

TABLE 7
Characteristics of Exosomes

| | Size (NTA) | Particle Yield |
|---|---|---|
| Colostrum Bovine Exo | 120 ± 15 | 3.46 ± 2.21E+13 |
| Raw Bovine Milk Exo | 121 ± 25 | 2.27 ± 1.73E+13 |
| Skim Bovine Milk Exo | 104 ± 5 | 5.13 ± 2.27E+13 |
| Goat Milk Exo | 91 ± 1 | 8.93 ± 1.94E+12 |

### Isolation Using Size Exclusion Chromatography

Excess fats were removed from raw milk prior to purification by size exclusion chromatography. Milk was first centrifuged at 13,000 RCF for 30 minutes at 4° C. in a Optima XE-90 ultracentrifuge. After completion of centrifugation step, the supernatant was decanted away from the pellet and subsequently filtered to remove any remaining solids. Filtered supernatant was mixed with phosphate-buffered saline (PBS) and centrifuged for a second time at 100,000 RCF for 71 minutes at 4° C. in a Optima XE-90 ultracentrifuge. The supernatant (whey fluid) was subsequently decanted from the pellet and concentrated using Amicon filtration at 3,500 rpm for 135 min with intermittent resuspension to a final volume of ˜8 mL. Whey fluid was then fractionated using either Sephacryl S500-HR or qEV chromatography resins.

Sephacryl S500-HR size exclusion columns were prepared by loading a suspension of Sephacryl S500-HR into a glass column and washing with excess PBS. The concentrated whey fluid was loaded onto the column and allowed to flow by gravity. PBS was used as an eluent and 1 mL fractions were collected.

qEV size exclusion columns were obtained from Izon Science (Medford, Mass., USA). Columns were rinsed with excess PBS prior to use. The concentrated whey fluid was loaded onto the column. PBS was used as an eluent and 1 mL fractions were collected.

Fractions were characterized using BCA protein assays, SDS-PAGE, and western blot analysis to identify those fractions which contained exosomes. Those fractions were subsequently pooled, concentrated, and made available for further experimental use.

Protein concentrations of exosomes prepared by SEC are shown in FIG. 1B. Results show that exosimes prepared by SEC are smaller with a higher concentration and yield as compared with exosomes prepared by UC.

### Example 2: Characterization of Exosomes

### Proteomics Analysis

Exosomes from bovine skim milk and goat milk were subjected to proteomics analysis. Isolated exosomes were sonicated for ten 1-second cycles using a probe sonicator at room temperature with an amount of RIPA buffer sufficient to disrupt the exosome and allow for proteins contained within the exosome to be released. 10 μg of each sample were then loaded onto a Bis-Tris NuPAGE gel for SDS-PAGE. The samples were run briefly and the migration window (˜1 cm lane) of each sample was excised. The excised in-gel samples were washed with 25 mM ammonium bicarbonate followed by acetonitrile. The in-gel samples were then reduced at 60° C. in 10 mM dithiothreitol before being alkylated at RT in 50 mM iodoacetamide. Trypsin was then added to samples and incubated for 4 hours at 37° C. in an orbital shaker to digest proteins. Digestion was quenched with 5% trifluoroacetic acid and samples were centrifuged. Digested peptides were individually loaded onto a trapping column and eluted over an analytical column that was interfaced to a Thermofisher Q Exactive Mass Spectrometer for peptide identification. The identified peptide data was then compared to known protein sequence databases to identify proteins present in bovine skim and goat milk exosomes.

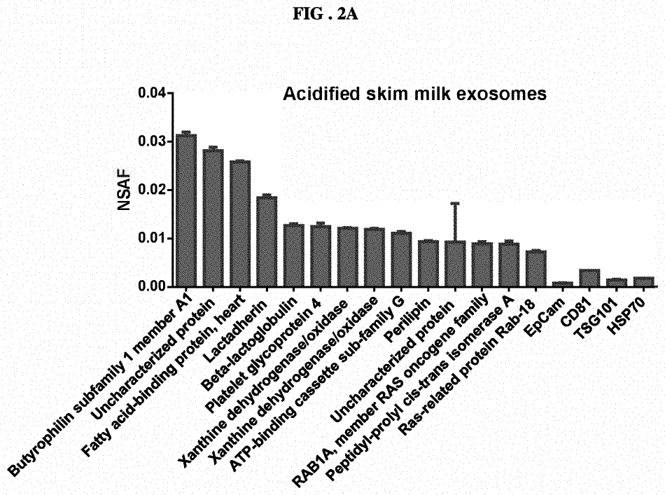

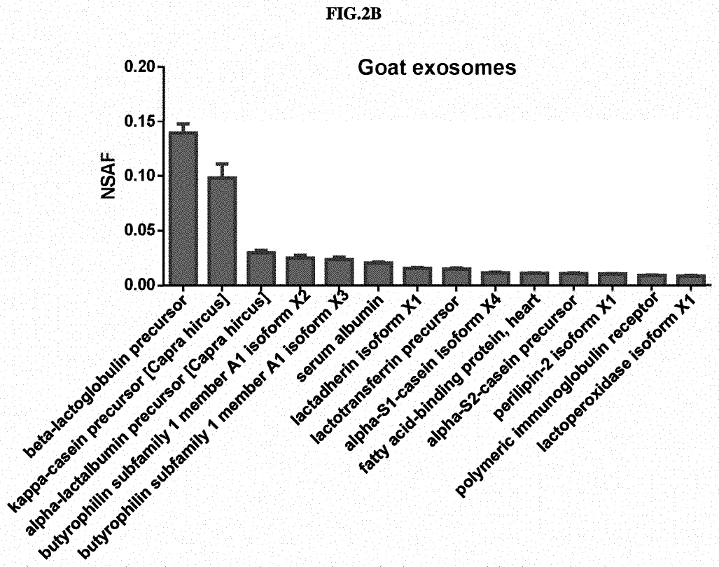

FIG. 2A and FIG. 2B show representative proteins identified in acidified skim milk exosomes and goat exosomes, respectively. Acidified milk refers to milk samples with casein removed by acid precipitation.

### Stability Analysis in Digestion Buffers

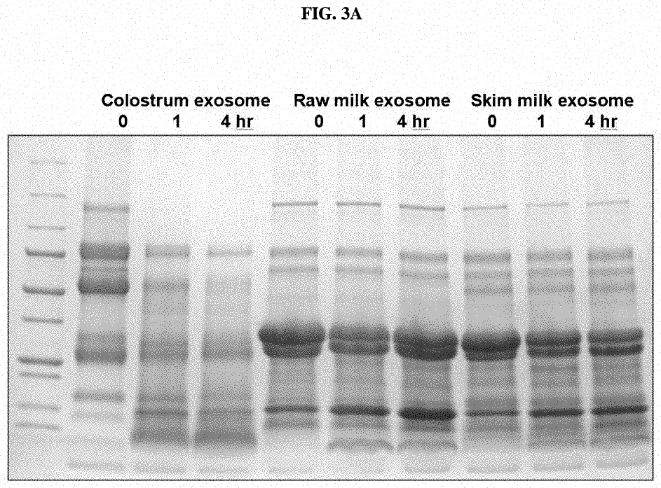

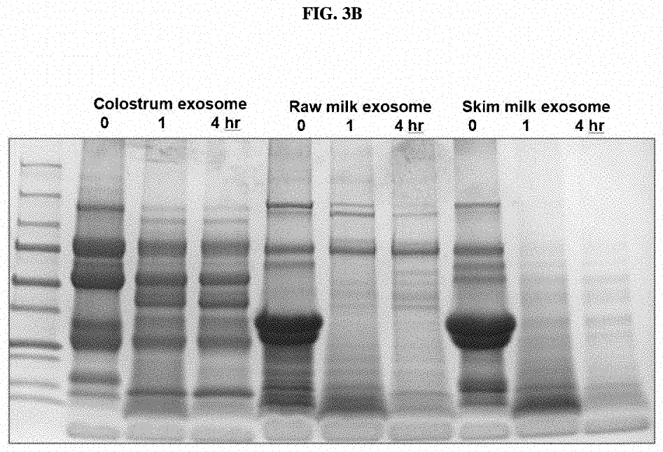

The stability of exosomes from raw bovine milk and goat milk was determined in different digestion buffers (Rat serum, Simulated intestinal fluid, Simulated gastric fluid, and phosphate buffer at pH of 2) over a time course (0, 1, 4 and 24 hours) by assessing a variety of protein profile and physical properties, including dynamic light scattering, SDS-PAGE and western blot analysis. Stability of exosomes from raw milk in response to being boiled was also evaluated. FIGS. 3A and 3B.

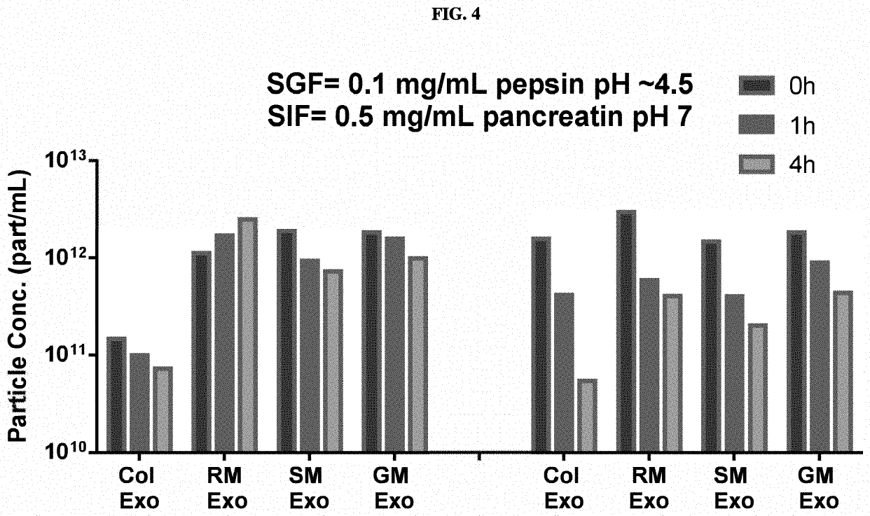

Previously isolated exosomes were diluted in PBS and aliquoted. Each aliquot was independently mixed with a different digestion buffer (Rat serum, simulated intestinal fluid, simulated gastric fluid, and phosphate buffer at pH of 2). Samples were then placed into an orbital shaker and incubated at 37° C. for 0, 1, 4 and 24 hours. An additional set of aliquots were boiled at 95° C. for 15 minutes in PBS. After their incubation period, each sample was mixed for 5 minutes at room temperature with an amount of RIPA buffer such that RIPA buffer represented ⅓ of the final volume. Samples were then ready for characterization. Results from this study are shown in FIG. 4.

Samples were analyzed by dynamic light scattering using a standard DLS instrument. Samples were analyzed by SDS-PAGE using a 4-20% Mini-PROTEAN® gel cassette (Bio-Rad Laboratories). Samples analyzed by SDS-PAGE were further subjected to western blot analysis using anti-CD81 and anti-CD47 antibodies.

### Stability Analysis in Intestinal Fluid

The stability of exosomes from skim milk, raw milk, and powdered colostrum milk was determined in intestinal fluid containing 0.5% pancreatin over a time course by assessing a variety of protein profile and physical properties, including dynamic light scattering, SDS-PAGE and western blot analysis.

Previously isolated exosomes were diluted in PBS, aliquoted in independent samples, and mixed with intestinal fluid containing 0.5% pancreatin. Samples were then placed into an orbital shaker and incubated at 37° C. for 0, 1, 4 and 24 hours. After their incubation period, each sample was mixed for 5 minutes at room temperature with an amount of RIPA buffer such that RIPA buffer represented ⅓ of the final volume. Samples were then ready for characterization.

Samples were analyzed by dynamic light scattering using a standard DLS instrument. Samples were analyzed by SDS-PAGE using a 4-20% Mini-PROTEAN® gel cassette (Bio-Rad Laboratories). Samples analyzed by SDS-PAGE were further subjected to western blot analysis using a cocktail of anti-CD81, anti-Alix, and anti-TSG101 antibodies; an anti-CD63 antibody; and/or an anti-EpCAM antibody. See FIG. 4.

### Nanoparticle Tracking Analysis (NTA)

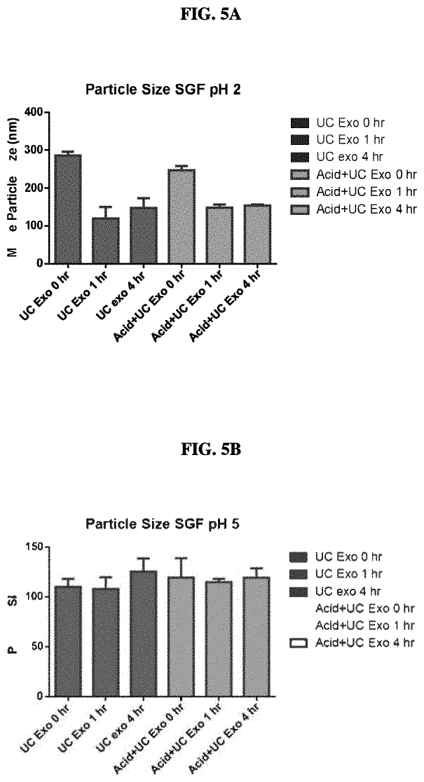

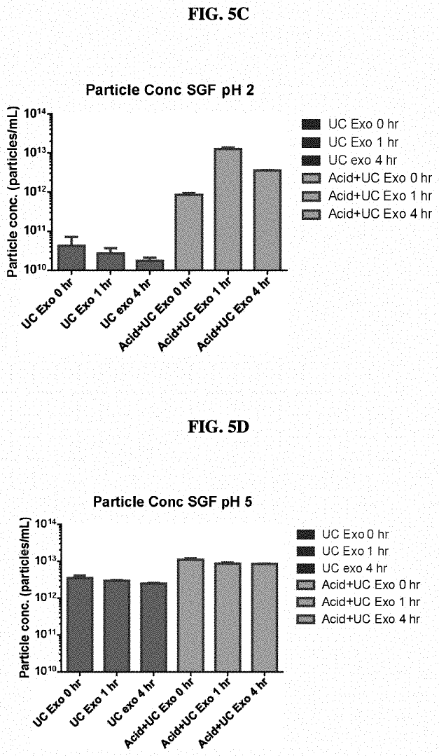

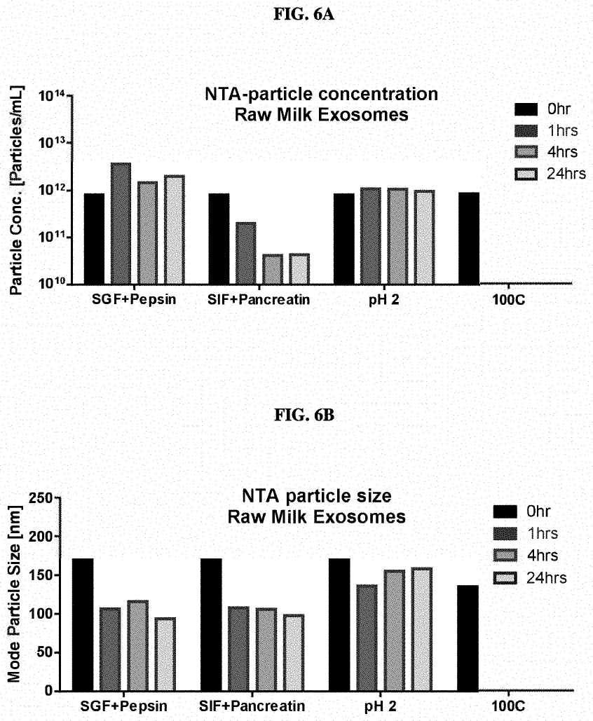

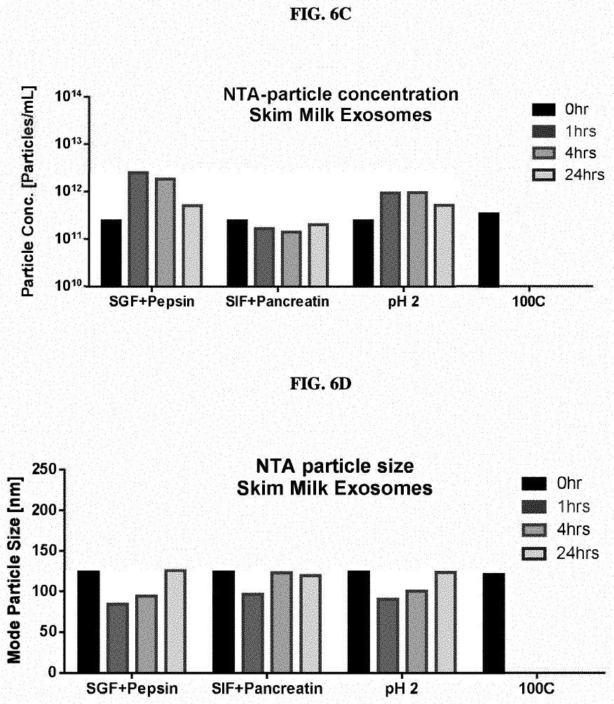

Nanoparticle tracking analysis (NTA) was performed to measure particle size and particle concentration. NTA is a method for visualizing and analyzing particles in liquids that relates the rate of Brownian motion to particle size. The results are shown in FIGS. 5A-5D and FIGS. 6A-6D.

Particle size and concentration were relatively unchanged for raw milk exosomes inducated with SGF and pepsin, under pH 2, and heated to 100° C. FIGS. 6A-6B. Similalry, particle size and concentration were relatively unchanged for skim milk exosomes inducated with SGF and pepsin, with SIF and pancreatin, under pH 2, and heated to 100° C. FIGS. 6C-6D.

### Example 3: Preparation of Milk Vesicles Involving Casein Removal Via Acidification

Conventional approaches of isolating exosomes from milk are typically based on ultracentrifugation. This example provides an exemplary method for isolating milk vesicles from milk that involves removal of casein via acidification. Also provided herein are characterization of milk vesicles thus obtained relative to milk vesicles obtained from the conventional ultracentrifugation method.

### Preparation Methods

Differential Ultra-centrifugation (UC) was performed as follows to separate the desired milk vesicles from a bulk solution. A milk sample was spun at 20,000×g for up to 30 minutes followed by multiple spins at ranges of about 100,000×g to about 130,000×g for about 1 to about 2 hours. The pellets thus produced, which are rich in milk-derived vesicles, are collected.

An exemplary casein removal by acidification followed by ultracentrifugation (AUC) method was performed as follows. Fresh raw milk was defatted using centrifugation 7-20 k g for 20-40 minutes. Acetic acid was then added to the defatted milk sample to coagulate casein. Casein precipitate was removed, and milk exosomes (extracellular vesicles or EV) were isolated using several centrifugation steps at ranges of about 100,000×g to about 130,000×g for about 1 to about 2 hours. The final pellet enriched in milk-derived vesicles was resuspended into PBS.

An exemplary disruption of casein micelles by EDTA followed by ultracentrifugation (EUC) method was performed as follows. Fresh raw milk was defatted using centrifugation 7-20 k g for 20-40 minutes. An EDTA solution was then added to the defatted milk sample to disrupt casein micelles. EVs were isolated using several centrifugation steps at ranges of about 100,000×g to about 130,000×g for about 1 to about 2 hours. The final pellet enriched in milk-derived vesicles was resuspended into PBS.

An exemplary casein removal by acidification followed by tangential flow filtration and size exclusion chromatography (ATFF/SEC) method was performed as follows. Fresh raw milk was defatted using centrifugation 7-20 k g for 20-40 minutes. Acetic acid was then added to the defatted milk sample to coagulate casein. Casein precipitate was removed and EVs were washed and concentrated using tangential flow filtration. Permeate thus formed was further purified using size exclusion chromatography using Sephacryl resin. The resultant milk vesicles were collected and resuspended into PBS.

### Protein Content Characterization

To analyze protein profile of different exosomes (EV), samples (each containing 20 μg of protein) were mixed with 4× Laemmli buffer (with 10% mercaptoethanol) and denaturated at 95° C. for 5 min. Then the samples were resolved using a standard SDS-PAGE procedure and gel was stained with SimplyBlue Coomassie stain for protein detection.

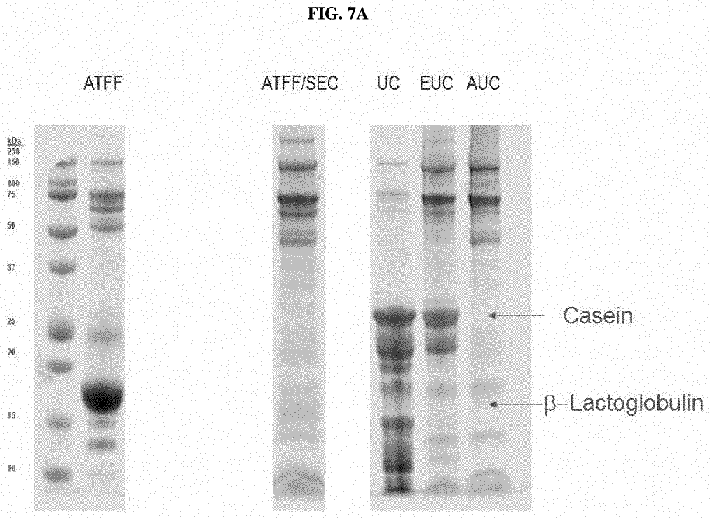

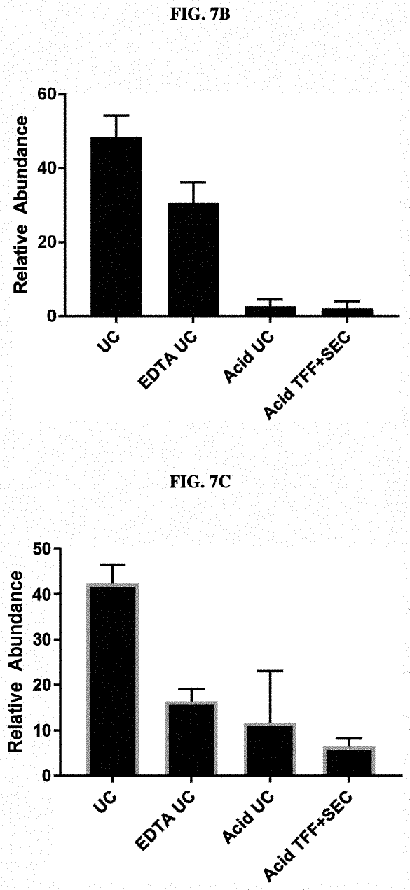

As shown in FIG. 7A, exosomes (EVs) isolated using either AUC or ATFF/SEC methods have similar protein profiles and are mainly depleted of major protein contaminations comparing to the UC or EUC methods.

The gel scans were analyzed to assess relative abundance of two major contaminant proteins groups, caseins and lactoglobulins. Briefly, 25-30 kDa band (mainly casein in bovine milk derived samples) was quantified using Coomassie staining of SDS-PAGE gel. The gel scan was quantified using ImageJ according standard procedure and normalized by the total signal in each lane. 10-20 kDa bands (mainly comprised of lactoglobulins in milk derived samples) were quantified using Coomassie staining of SDS-PAGE gel. The gel scan was quantified using ImageJ according standard procedure and normalized by the total signal in each lane. Both caseins and lactoglobulins were efficiently depleted by the AUC and ATFF/SEC methods by 4-5 folds comparing to the conventional UC method. FIGS. 7B and 7C. The relative abundance of caseins and lactoglobulins in EV compostions prepared by the EUC method was also significantly lower than that in EV compositions prepared by the conventional UC method.

### Example 4: Preparation of Milk Vesicles Involving Casein Removal Via Coagulation with Vegetable Rennet

To compare the effect of different methods of casein isolation on protein profiles of the resultant exosomes (EV) and presence of exosome markers, relative abundance of milk exosomes and milk exosome markers were analyzed in milk exosomes prepared by ATFF/SEC and VRTFF/SEC methods.

ATFF/SEC was performed as described in Example 3 above. An exemplary casein removal by coagulation with vegetable rennet followed by tangential flow filtration and size exclusion chromatography method (VRTFF/SEC) was carried out as follows. Casein was coagulated in raw milk (or defatted milk) using vegetable rennet. Coagulated casein was removed following the standard procedure. The resultant EVs were washed and concentrated using tangential flow filtration. The permeate was further purified using size exclusion chromatography and the resultant EV composition was collected.

EV isolates were mixed with 4× Laemmli buffer (with 10% mercaptoethanol) and incubated at 95° C. for 5 min. The samples were then resolved using a standard SDS-PAGE procedure and transferred to PVDF membrane. The membranes were then blotted using anti-CD81, anti-BTN1A1, or anti-XOR antibodies.

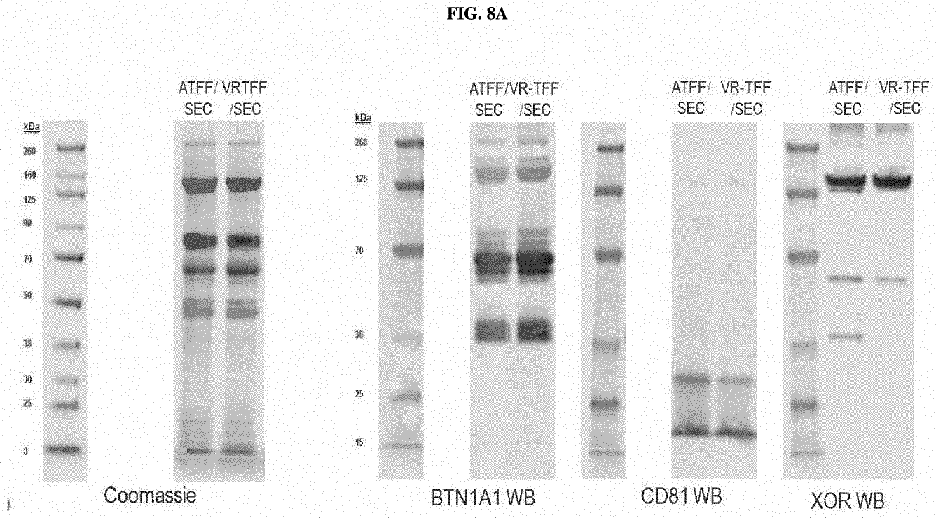

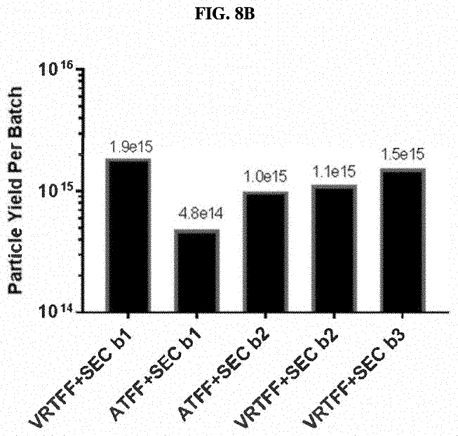

As shown in FIG. 8A, rennet coagulation of casein did not lead to loss or degradation of exosome (EV) and milk-exosome (MEV) specific markers.

To assess the effect of removing casein using rennet enzyme mixture on the yield of milk exosomes, final concentration of exosomes prepared by VRTFF/SEC and ATFF/SEC were measured by Nanosight Tracking Analysis and recalculated to the volume of input material. EV isolates were diluted in 0.1 um filtered 1×PBS and ran using a standard Nanosight Tracking Analysis (NTA) protocol to determine particle concentration and size. Particle yield was determined by multiplying the particle concentration by the volume of EV isolate. As shown in FIG. 8B, both ATFF/SEC (removing casein by acidification) and VRTFF/SEC (removing casein by vegetable rennet) lead to similar particle yields.

### Example 5: Characterization of Milk Vesicles

Various features of milk vesicle compositions prepared by the methods disclosed herein (see, e.g., Examples 3 and 4 above) were analyzed. Some examples are provided below.

(i) Biomarkes of Milk Vesicles

To assess whether conventional exosome markers (e.g., CD81) are present in BTN1A1 poitive particles, a co-immunoprecipitation assay was performed. Briefly, milk EVs were incubated with varying amounts of anti-BTN1A1 antibodies and pulled down using protein A magnetic beads. The unbound materials were washed off the bound materials and the lysed bound materials were analyzed by standard western blotting procedure using anti-BTN1A1 and anti-CD81 antibodies.

This assay indicates that CD81-related signal is enriched in BTN1A1 positive particles, suggesting that milk EVs prepared by the methods disclosed herein (e.g., involving casein removal as exemplified in Examples 3 and 4 above) may have presence of both BTN1A1 and CD81. FIG. 9.

(ii) Milk Exosomes Tolerate Freeze-Thaw Cycles and Temperature Treatment

Casein depleted exosomes prepared via ATFF/SEC were assessed for stability at different temperature conditions as well as for their resistance in freeze thaw cycles as follows.

The exosomes (EVs) were isolated from milk with casein depleted using acid-promoted coagulation and filtration through cheesecloth followed by TFF as described above. The TFF isolated EVs were subsequently purified via size exclusion chromatography (SEC) using Sephacryl resin. The particle concentration of the EV stock solution was 4×10¹² particles/ml. EV stock was mixed with 100 mM Trehalose/PBS or PBS in 1:1 ratio for final particle concentration of 2×10¹² particles/ml. The samples were stored at: −80° C. for 24 h, 4° C. for 24 h, room temperature for 96 h, 60° C. for 40 min, or 100° C. for 10 min. In addition, one sample of EVs in PBS and one sample in 50 mM Trehalose/PBS were subjected to 6 freeze-thaw cycles. Each freeze-thaw cycle was conducted by placing the samples in dry ice for 5 min followed by incubation at 37° C. for 5 min.

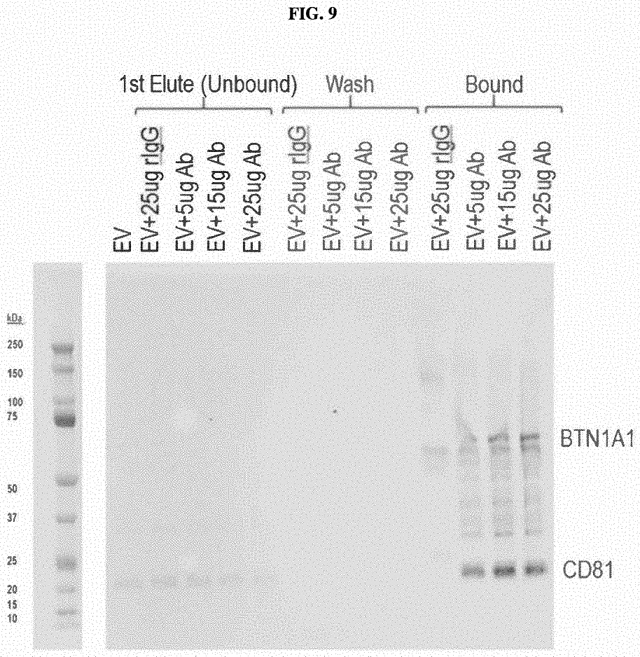

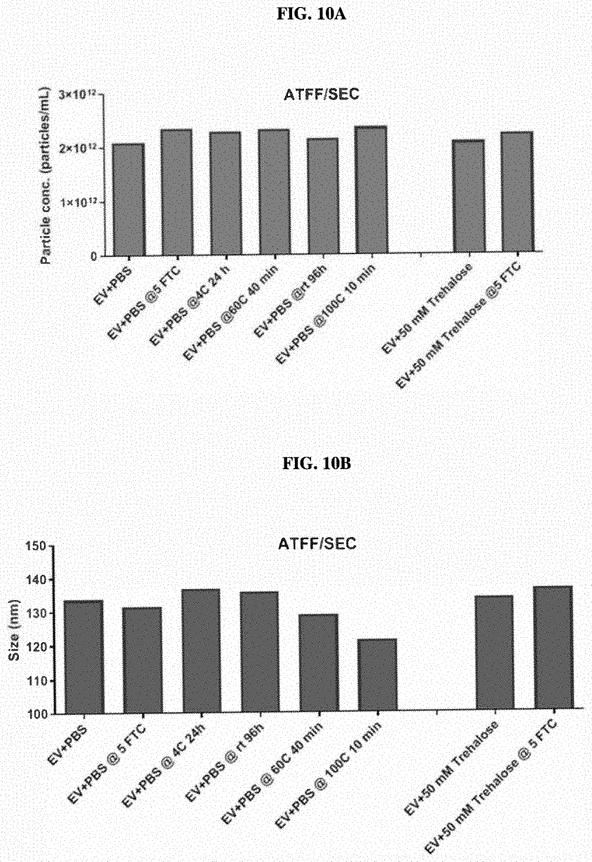

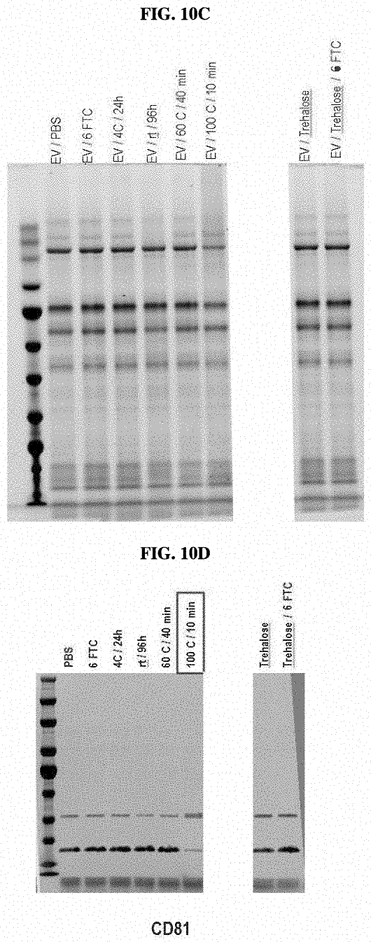

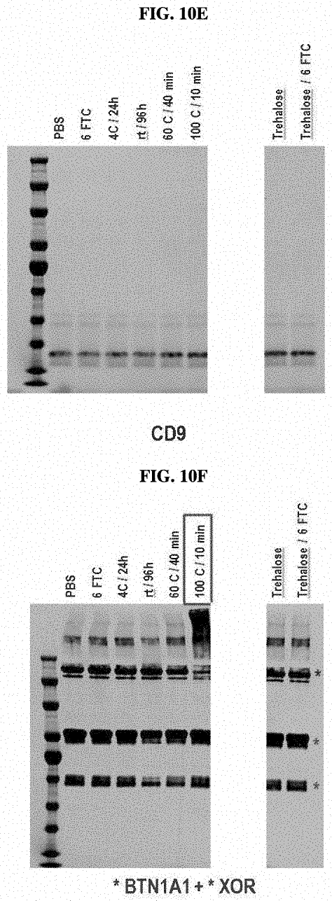

Particle size and concentration were measured using the Malvern Nanosight NS300 Nanoparticle Tracking Analysis (NTA) instrument. Briefly, all samples measured were diluted in 0.1 μm filtered 1×PBS. Each sample was injected via 1 ml syringe into the instrument using a syringe pump set at flow rate 30. The particle flow for each sample was recorded for 5×30 s using camera level 14 and analyzed using level 5 setting. No treatment or storage condition led to change in particle concentration and only treatment at 100° C. for 10 min led to reduction in particle size. FIGS. 10A and 10B.

Further, protein profiles of the samples were analyzed after being incubated at the differen temperature conditions or the freeze-thaw cycles as disclosed above via SDS-PAGE analysis. Briefly, each sample was mixed with 4× Laemmli buffer (with 10% mercaptoethanol) and incubated at 95° C. for 5 min. The samples were analyzed for protein content on a 4-12% NuPAGE MIdi Gel run on a XCell Surelock MidiCell at 200 V. The proteins were visualized using SimplyBlue SafeStain. The stained gel was imaged using the Licor CLx Oddissey imaging system (700 nm laser). No treatment or storage condition led to change in protein profile of particles isolated by ATFF/SEC. FIG. 10C.

In addition, Western blot analysis was performed to examine levels of milk vesicle biomarkers after the temperature treatment or the freeze-thaw cycles. Briefly, exosome (EV) isolates were mixed with 4× Laemmli buffer (with 10% mercaptoethanol) and incubated at 95° C. for 5 min. The samples were resolved using a standard SDS-PAGE procedure and transferred to PVDF membrane. The membranes were then blotted using anti-CD81, anti-BTN1A1, or anti-XOR antibodies. Most treatment or storage condition did not lead to significant change of BTN1A1 and XOR abundance. Boiling for 10 min led to reduction of CD81 and XOR levels. FIGS. 10D to 10F.

(iii) Colloidal Stability of Milk Exosomes Loaded with Cholesterol-Modified siRNA

Colloidal stability refers to the long-term integrity of a dispersion and its ability to resist phenomena such as sedimentation or particle aggregation. This is typically defined by the time that dispersed phase particles can remain suspended. To test colloidal stability of milk EV in the presence of cholesterol-modified oligonucleotide (“chol-ON”), EVs isolated from milk via the conventional ultracentrifugation approach without casein removal (“UC”) or using tangential flow filtration followed by size exclusion chromatography with casein removal through acidification as disclosed herein (ATFF/SEC) were incubated with cholesterol-siRNA-DY677 for 1.5 h at room temperature in a ratio of 5000/1 siRNA/EV. The samples were left at 4° C. for 24 h. EVs from colostrum powder isolated via ultracentrifugation (UC) without casein removal were incubated with cholesterol-siRNA-DY677 for 1.5 h at room temperature in ratios of 500/1, 1000/1, or 5000/1 siRNA/EV. The samples were kept at 4° C. for 24 h. The results indicate that casein-containing milk EV isolates as well as colostrum derived EV isolates lost colloidal stability when incubated with cholesterol modified siRNA. By contract, milk EV isolated prepared by the ATFF/SEC method maintained colloidal stability.

Further, milk EV isolates prepared by the conventional US approach and those prepared by the ATFF/SEC method disclosed herein were incubated with chol-ON for 1.5 h at room temperature in a ratio of 0/1, 5000/1 and 20000/1 chol-ON/EV. The samples were left at 4° C. for 24 h and centrifuged at 2000 g for 2 min to collect and visualize the precipitate, if formed. The results thus obtained show that casein-containing milk EV isolates lose colloidal stability when incubated with cholesterol modified ON—precipitates were visible after the centrifugation. By contrast, no precipitates were observed in the milk EV sample prepared by the ATFF/SEC method after the centrifugation, indicating that the milk EVs prepared by the ATFF/SEC method maintained colloidal stability.

(iv) Removal of Casein does not Affect Particle Stability in Simulated Gastric Fluid

Further, stability of milk exosomes prepared with or without casein removal at low pH was analyzed. UC or AUC exosomes were incubated with simulated gastric fluid at pH 2 or 5 for 0-4 hours. Particle size and concentration were measured using the Malvern Nanosight NS300 NTA instrument. All samples measured were diluted 20,000× in 0.1 um filtered 1×PBS. Each sample was injected via 1 ml syringe into the instrument using a syringe pump set at flow rate 30. The particle flow for each sample was recorded for 5×30 s using camera level 14 and analyzed using level 5 setting.

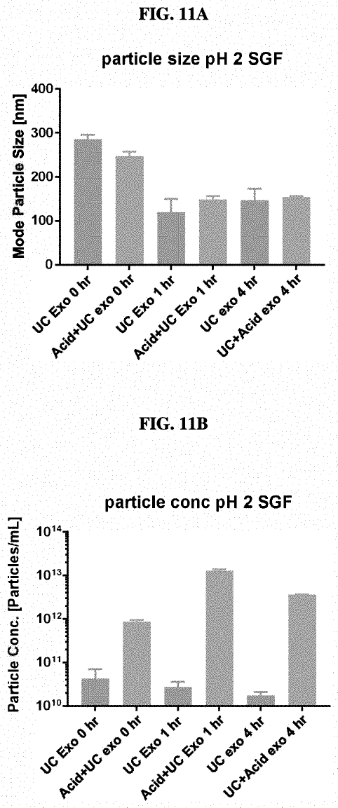

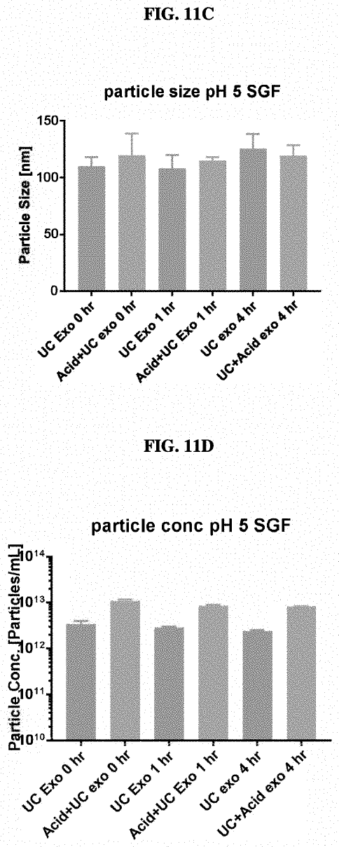

As shown in FIGS. 11A-11D, both UC and AUC exosomes tolerate well incubation at low pH without significant loss of particles.

(v) Loading Capacity of Casein-Depleted Milk Vesicles

To assess loading efficiency of cholesterol-modified oligonucleotides to casein-depleted milk vesicles, EVs from milk isolated using tangential flow filtration followed by size exclusion chromatography with casein removal through acidification (ATFF/SEC) were incubated with cholesterol-siRNA-Cy5.5 for 1.5 h at room temperature in ratio of 600/1, 1200/1, 2400/1, or 4800/1 siRNA/EV. All samples were purified using 2 ml Zeba spin columns with 40 kDa cutoff to remove unbound siRNAs. The absorbance and fluorescence spectra of all samples were measured before and after the purification. The loading % was calculated using the ratio of the area under the curve before and after purification for both absorbance and fluorescence of the Cy5.5 dye.

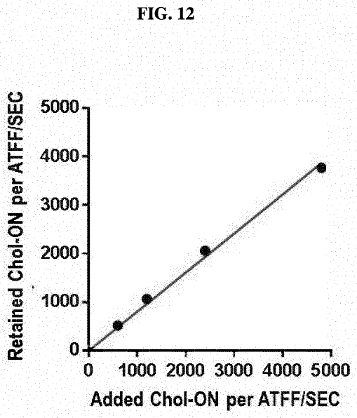

The results indicate that incubation with various concentrations of cholesterol-modified siRNA with ATFF/SEC EV allowed efficient loading of at least ˜5000 siRNA per exosome. FIG. 12. The loading capacities under the various siRNA/EV ratios are provided in Table 8 below.

TABLE 8
Loacing Capacity of Casein-Depleted Milk Vesicles

| ON/MEV | Loading, % |
|---|---|
| 600/1 | 87.1 |
| 1200/1 | 89.2 |
| 2400/1 | 85.9 |
| 4800/1 | 78.4 |

(vi) Casein-Depleted Milk Vesicles Protect Oligonucleotide from S1 Nuclease Digestion

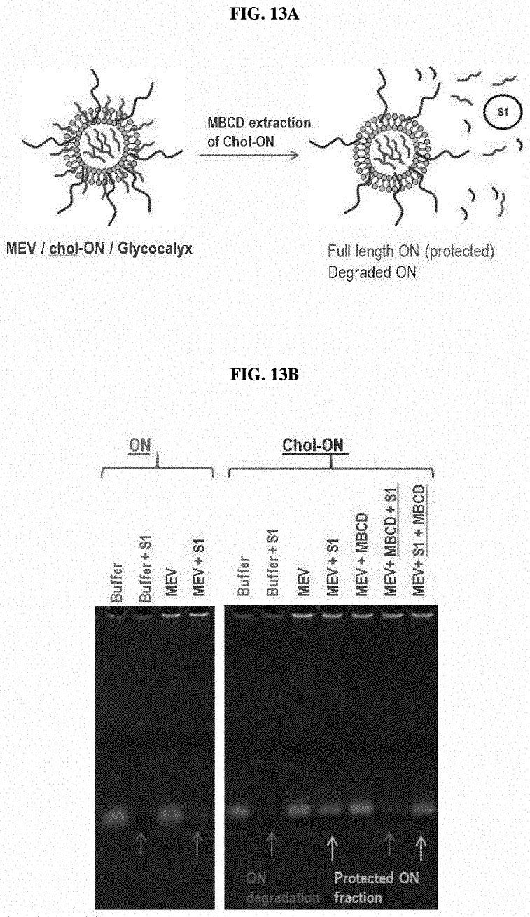

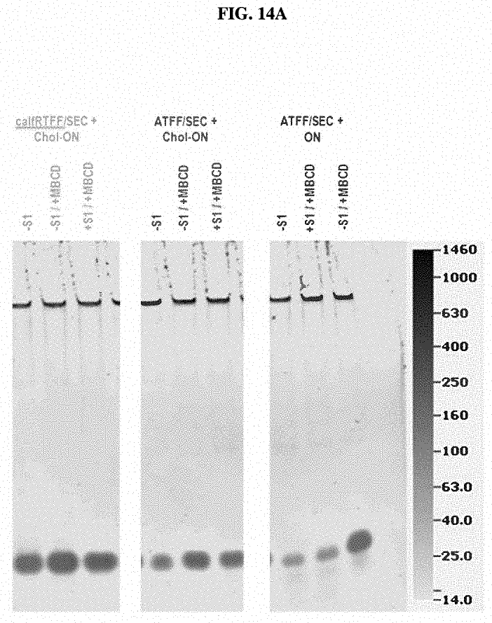

Nuclease-protection of loaded oligonucleotide was protected using input material with three methods of casein removal: (i) acidification-promoted casein coagulation, (ii) calf rennet-promoted casein coagulation, and (iii) vegetable rennet-promoted casein coagulation. FIG. 13A illustrates an exemplary process for assessing EV protection of loaded oligonucleotides. Briefly, chol-ON was incubated for 1.5 h at room temperature with EVs isolated from milk using tangential flow filtration followed by size exclusion chromatography with casein depletion via the (i), (ii), and (iii) approaches noted above at a ratio of 600/1, 350/1 and 350/1 (chol-ON/EV), respectively.

The S1 nuclease (Aspergillus oryzae) degradation assay was conducted in acetate buffer pH=4.6 (60 mM NaOAc, 1 mM ZnSO4). Each oligonucleotide (ON) sample (with or without cholesterol) in buffer or in EVs (milk EV isolated using tangential flow filtration followed by size exclusion chromatography with casein depletion through the (i), (ii), or (iii) approaches was split into 2 aliquots. To one aliquot, S1 nuclease was added at a final nuclease concentration of 10 U/ul and the other was supplemented with the same amount of buffer (60 mM NaOAc, 1 mM ZnSO4, pH=4.6) as a blank control. All samples were incubated for 45 min at 37° C. Each ON/EV sample was then split into 3 aliquots. To one of the aliquots, 20 mM Methyl beta cyclodextrin (MBCD) was added and incubated for 5 min and the reaction was quenched with 30 mM EDTA. This aliquot was then heated to 85° C. for 5 min to deactivate the S1 nuclease. To the other 2 aliquots, either PBS buffer or 30 mM MBCD in PBS were added after the reaction was quenched. All samples were incubated for 10 min at room temperature; analyzed on 20% TBE PAGE and run at 200 V using XCell SureLock™ Mini-Cell. The gel was stained with SYBR Gold (10,000× in TBE buffer) for 10 min on a shaker at 4° C. The gel was imaged using a boxed UV light to visualize the dye.

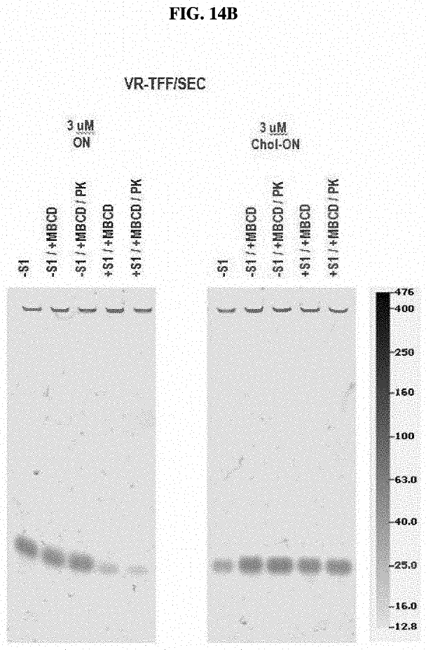

As shown in FIG. 13B and FIGS. 14A-14B, milk vesicles prepared by methods involving casein depletion, including approaches (i)-(iii) noted above, protected the loaded oligonucleotides from S1 nuclease digestion. Efficiency of the protection is provided in Table 9 below.

TABLE 9
Quantitation of nuclease protection effect by
ATFF/SEC, calfRTFF/SEC, and VRTFF/SEC Milk EVs

| | Nuclease Protection % |
|---|---|
| calfR/TFF/SEC + Chol-ON | 90.9 |
| VRTFF/SEC + Chol-ON | 85 |
| ATFF/SEC + Chol-ON | 71.6 |
| ATFF/SEC + ON | 20.5 |

(vii) Pegylated Liposomes do not Protect Cholesterol-Modified Oligonucleotides from S1 Nuclease Digestion

To assess the specificity of cholesterol-ON protection, pegylated liposomes were prepared according to the standard protocol. Briefly, DOPC/DOPE/DPPE-PEG2000 was mixed in 49.5/49.5/1 mol % in a 2 dram glass vial. The lipids were dissolved in 100 ul chloroform, which was evaporated under a stream of air while the vial was manually rotated in order to form a thin film on the walls of the vial. The lipid film was dried under vacuum for 1 h to remove trace amounts of chloroform. The lipid film was hydrated with Cholesterol-ON-DY677 80 mM NaOAc buffer pH=4.5. The suspension was vortexed for 5 min followed by extrusion using the Avanti Polar Lipids extruder with 100 nm Polycarbonate Membranes. The mixture was passed 11 times through the extruder. The resultant sample was purified using 2 ml Zeba desalting spin columns with 40 kDa cutoff.

Cholestrol-ON-DY677 was incubated for 1.5 h at room temperature with EVs isolated from milk using tangential flow filtration followed by size exclusion chromatography with casein depletion via acidification (ATFF/SEC) at the ratio of 600/1 (ON/EV).

The S1 nuclease (Aspergillus oryzae) degradation assay was conducted in acetate buffer pH=4.6 (80 mM NaOAc, 5 mM ZnSO₄). Each ON-DY677 sample (with or without cholesterol) in EVs (ATFF/SEC), liposomes, or buffer was split into 2 aliquots. To one aliquot, S1 nuclease was added for final nuclease concentration of 20 U/ul and the other was supplemented with the same amount of buffer (80 mM NaOAc, 5 mM ZnSO4, pH=4.6) as a blank control. All samples were incubated for 45 min at 37° C. and quenched with 30 mM EDTA. The samples were heated to 85° C. for 5 min to deactivate the S1 nuclease. Each sample was split into 2 aliquots and either PBS or 30 mM MBCD in PBS was added to one aliquot after the reaction was quenched. All samples were incubated for 10 min at room temperature. The samples were then analyzed on 20% TBE PAGE and run at 200 V using XCell SureLock™ Mini-Cell. The gel was imaged using Licor CLx Oddissey imaging system (700 nm channel).

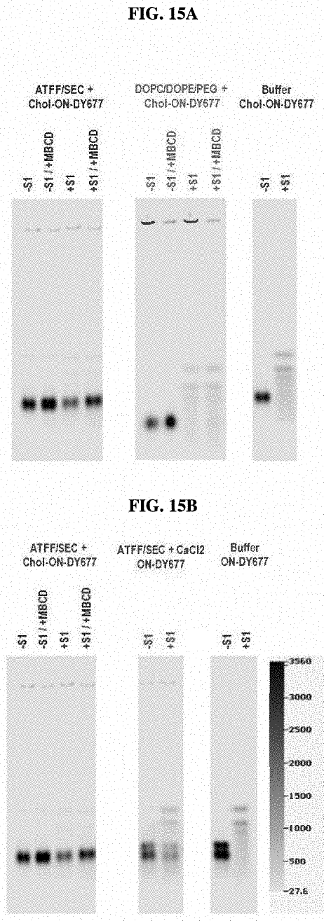

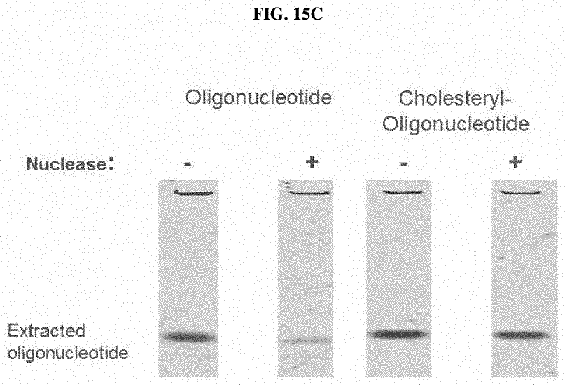

As shown in FIG. 15A, PEGylated liposomes do not protect cholesterol-ON in the 51 nuclease digestion assay, contrary to casein depleted milk EV. Protectin efficiency is provided in Table 10 below.

TABLE 10
Quantitation of nuclease protection effect
by ATFF/SEC Milk EVs and Liposomes

| +20 U/ul S1 | Nuclease Protection % |
|---|---|
| ATFF-SEC Chol-ON-DY677 | 72.1 |
| DOPC/DOPE/PEG Chol-ON-DY677 | 3.3 |
| Chol-ON-DY677/Buffer | 0.5 |

Table 11 below shows relative protection efficiency of modified or non-modified oligonucleotides (ON) in nuclease digestion assays.

TABLE 11
Relative Protection Efficiency in Nuclease Assays

| | Relative protection |
|---|---|
| | in nuclease assay, % |
| Non-modified ON | 13.1 |
| Cholesteryl ON | 74.3 |
| Tocopheryl ON | 105.4 |
| Palmitoyl ON | 54.1 |

(viii) Calcium/Ethnol Precipitation of Oligonucleotides does not Lead to Efficient Protection from S1 Nuclease Digestion

This study further compares efficiency of protection for cholesterol-ON loaded to exosomes vs oligonucleotides transfected using conventional transfection protocol (i.e., Ca/Ethanol transfection).

ON-DY677 were incubated for 1.5 h at room temperature with EVs isolated from milk using tangential flow filtration followed by size exclusion chromatography with casein depletion via acidification (ATFF/SEC) at the ratio of 600/1 (ON/EV). Alternatively, ON-DY677 was transfected using CaCl₂/40% ethanol into milk EVs isolated via tangential flow filtration followed by size exclusion chromatography with casein depletion via acidification (ATFF/SEC). The EVs were mixed with the ON at the ration of 600/1 (ON/EV) after CaCl₂ and Ethanol were added. The sample was incubated at room temperature for 1.5 h. All samples were purified via ultracentrifugation at 135,000 g for 104 min at 4° C. and then resuspended in PBS by vortexing.

The S1 nuclease (Aspergillus oryzae) degradation assay was performed following the descriptions provided above. Contrary to cholesterol-ON, Ca/Ethanol transfected ON are not protected by milk exosomes in the nuclease protection assay. FIGS. 15A and 15B. The loading and nuclease protection efficiencies are shown in Table 12 below.

TABLE 12
Loading Efficiency and Nuclease Protection efficiency

| | Loading % |
|---|---|
| ATFF/SEC + ON-DY677 | NA |
| ATFF/SEC + Chol-ON-DY677 | 88.7 |
| ATFF/SEC + ON-DY677 + CaCl₂ | 99.0 |

| +20 U/ul S1 | Nuclease Protection % |
|---|---|
| ATFF/SEC Chol-ON-DY677 | 72.1 |
| ATFF/SEC ON-DY677 + CaCl₂ | 47.2 |
| Chol-ON-DY677/Buffer | 0.5 |
| ON-DY677/Buffer | 0.1 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A composition comprising milk vesicles, wherein the milk vesicles comprise a lipid membrane to which one or more proteins are associated, and wherein (a) a relative abundance of casein in the composition is less than about 40%, and/or (b) a relative abundance of lactoglobulin in the composition is less than about 25%; and wherein the milk vesicles are loaded with a cargo.

2. The composition of claim 1, wherein the relative abundance of casein in the composition is less than about 20%.

3. The composition of claim 2, wherein the relative abundance of casein in the composition is less than about 5%.

4. The composition of claim 3, wherein the composition is substantially free of casein.

5. The composition of claim 1, wherein the relative abundance of lactoglobulin is less than about 15%.

6. The composition of claim 5, wherein the relative abundance of lactoglobulin is less than 10%.

7. The composition of claim 1, wherein the cargo is a biological molecule not naturally-occurring in the milk vesicle.

8. The composition of claim 1, wherein the size of the milk vesicles is about 20-1,000 nm.

9. The composition of claim 8, wherein the size of the milk vesicles is about 80-200 nm.

10. The composition of claim 9, wherein the size of the milk vesicles is about 120-160 nm.

11. The composition of claim 1, wherein the one or more proteins associated with the lipid membrane of the milk vesicles comprise Butyrophilin Subfamily 1 Member A1 (BTN1A1) or a transmembrane fragment thereof, Butyrophilin Subfamily 1 Member A2 (BTN1A2) or a transmembrane fragment thereof, fatty acid binding protein, lactadherin, platelet glycoprotein 4, xanthine dehydrogenase, ATP-binding cassette subfamily G, perillipin, RAB1A, peptidyl-prolyl cis-transisomerase A, Ras-related protein Rab-18, EpCAM, CD63, CD81, TSG101, HSP70, lactoferrin or a transmembrane fragment thereof, ALG-2-interacting protein X, alpha-lactalbumin, serum albumin, polymeric immunoglobulin, lactoperoxidase, or a combination thereof.

12. The composition of claim 11, wherein the milk vesicles comprise BTN1A1 and CD81.

13. The composition of claim 11, wherein the one or more proteins associated with the lipid membrane of the milk vesicles comprise glycosylated proteins.

14. The composition of claim 1, wherein the milk vesicles are obtained from cow milk, goat milk, camel milk, buffalo milk, yak milk, or human milk.

15. The composition of claim 1, wherein the milk vesicles are selected from the group consisting of lactosome, milk fat globule (MFG), exosome, extracellular vesicles, whey-particle, whey-derived particle, aggregates thereof, and combinations thereof.

16. The composition of claim 7, wherein the biological molecule is a peptide, a protein, a nucleic acid, a polysaccharide, or a small molecule.

17. The composition of claim 16, wherein the biological molecule is a protein selected from the group consisting of an antibody, a hormone, a growth factor, an enzyme, a cytokine, a chemokine, a toxin, an antitoxin, a blood coagulation factor, or a combination thereof.

18. The composition of claim 16, wherein the biological molecule is a nucleic acid selected from the group consisting of an interfering RNA (iRNA), a microRNA (miRNA), an antisense RNA, a messenger RNA (mRNA), a non-coding RNA, a single-stranded DNA (ssDNA), a double-stranded DNA (dsDNA), or a combination thereof.

19. The composition of claim 18, wherein the iRNA is siRNA or shRNA.

20. The composition of claim 7, where the biological molecule is conjugated to a hydrophobic moiety.

21. The composition of claim 20, wherein the hydrophobic moiety is selected from the group consisting of a lipid, a sterol, a steroid, a terpene, cholic acid, adamantine acetic acid, 1-pyrene butyric acid, 1,3-bis-O(hexadecyl)glycerol, a geranyloxyhexyl group, hexadecylglycerol, borneol, 1,3-propanediol, heptadecyl group, O3-(oleoyl)lithocholid acid, O3-(oleoyl)cholenic acid, dimethoxytrityl, a phenoxazine isoprene derivative, tocopherol and tocotrienol.

22. The compositions of claim 1, wherein the milk vesicles comprise one or more of the following features:
(i) stability under freeze-thaw cycles and/or temperature treatment;
(ii) colloidal stablility when the milk vesicles are loaded with the biological molecule;
(iii) a loading capacity of at least 5000 cholesterol modified oligonucleotides per milk vesicle;
(iv) stablility under acidic pH;
(v) stablility upon sonication;
(vi) resistance to enzyme digestion; and
(vii) resistance to nuclease treatment upon loading of the milk vesicles with oligonucleotides.

23. The composition of claim 22, wherein the acidic pH of (d) is ≤4.5.

24. The composition of claim 23, wherein the acidic pH of (d) is ≤2.5.

25. The composition of claim 22, wherein the enzyme digestion of (f) comprises digestion by one or more digestive enzymes.

26. The composition of claim 25, wherein the one or more digestive enzymes comprise protease, lipase, amylase, and/or nuclease.

27. The composition of claim 26, wherein the one or more digestive enzymes comprise lingual lipase, salivary amylase, pepsin, gastric lipase, trypsin, chymotrypsin, cardoxypeptidase, elastase, pancreatic lipase, phospholipase, DNAase, RNAase, pancreatic amylase, erepsin, maltase, lactase, and/or sucrose.

28. The composition of claim 27, wherein the one or more digestive enzymes comprise pepsin and pancreatin.

29. The composition of claim 1, wherein the composition is formulated into a pharmaceutical composition, which further comprises a pharmaceutically acceptable carrier.

30. The composition of claim 29, where the pharmaceutical composition is for oral administration.

31. A method for preparing a composition comprising milk vesicles, the method comprising:
(i) providing a first milk sample;
(ii) removing casein and/or lactoglobulin from the first milk sample to produce a second milk sample;
(iii) isolating milk vesicles from the second milk sample to produce a composition comprising the milk vesicles; and
(iv) loading a cargo to the milk vesicles.

32. The method of claim 25, wherein the first milk sample is from cow milk, goat milk, camel milk, buffalo milk, yak milk, or human milk.

33. The method of claim 31, wherein the first milk sample is raw milk, skim milk, colostrum, homogenized milk, or pasteurized milk.

34. The method of claim 31, wherein the removing step (ii) is performed by acidifying the first milk sample.

35. The method of claim 31, wherein the removing step (ii) is performed by coagulating the first milk sample with rennet.

36. The method of claim 35, wherein the rennet is animal rennet or plant rennet.

37. The method of claim 36, wherein the animal rennet is derived from calf intestine and/or wherein the plant rennet is vegetable rennet.

38. The method of claim 31, wherein the removing step (ii) is performed by disrupting casein micelles by EDTA.

39. The method of claim 31, wherein the isolating step (iii) is performed by ultracentrifugation, size exclusion chromatography, affinity purification, tangential flow filtration, or a combination thereof.

40-41. (canceled)

42. The of claim 31, wherein the cargo is a biological molecule not naturally-occurring in the milk vesicle.

43-48. (canceled)


SEQUENCE LISTING (22 sequences)

SEQ ID NO: 1 - RNA, 21 nt, Artificial Sequence
  Synthetic
  misc_feature (21)..(21): modified with phosphorothioate
gcacauaggagagaugagcuu

SEQ ID NO: 2 - RNA, 21 nt, Artificial Sequence
  Synthetic
  misc_feature (1)..(1): modified with phosphorothioate
  misc_feature (21)..(21): modified with phosphorothioate
gucaucacacugaauaccaag

SEQ ID NO: 3 - DNA, 21 bp, Artificial Sequence
  Synthetic
  misc_feature (21)..(21): modified with phosphorothioate
caucacacugaauaccaautt

SEQ ID NO: 4 - RNA, 22 nt, Artificial Sequence
  Synthetic
  misc_feature (1)..(22): modified with 2′-O-methyl
  misc_feature (1)..(2): modified with phosphorothioate
  misc_feature (19)..(22): modified with phosphorothioate
ucacgcgagccgaacgaacaaa

SEQ ID NO: 5 - DNA, 21 bp, Artificial Sequence
  Synthetic
  misc_feature (1)..(2): modified with 2′-O-methyl
  misc_feature (10)..(11): modified with 2′-O-methyl
  misc_feature (15)..(17): modified with 2′-O-methyl
  misc_feature (19)..(19): modified with 2′-O-methyl
  misc_feature (20)..(20): modified with phosphorothioate
cugggaaagucaagcccautt

SEQ ID NO: 6 - DNA, 21 bp, Artificial Sequence
  Synthetic
  misc_feature (1)..(2): modified with 2′-O-methyl
  misc_feature (4)..(4): modified with 2′-O-methyl
  misc_feature (6)..(6): modified with 2′-O-methyl
  misc_feature (10)..(10): modified with 2′-O-methyl
  misc_feature (12)..(14): modified with 2′-O-methyl
  misc_feature (19)..(19): modified with 2′-O-methyl
  misc_feature (21)..(21): modified with 2′-O-methyl
cugugcaagugcccaagautt

SEQ ID NO: 7 - DNA, 21 bp, Artificial Sequence
  Synthetic
  misc_feature (3)..(4): modified with 2′-deoxy-2′-fluoro
  misc_feature (6)..(9): modified with 2′-deoxy-2′-fluoro
  misc_feature (13)..(16): modified with 2′-deoxy-2′-fluoro
  misc_feature (18)..(18): modified with 2′-deoxy-2′-fluoro
  misc_feature (20)..(20): modified with phosphorothioate
ggaucaucucaagucuuactt

SEQ ID NO: 8 - DNA, 21 bp, Artificial Sequence
  Synthetic
  misc_feature (20)..(21): modified with phosphorothioate
acugcagggugaagaauuatt

SEQ ID NO: 9 - RNA, 21 nt, Artificial Sequence
  Synthetic
  misc_feature (20)..(21): modified with phosphorothioate
gcacauaggagagaugagcuu

SEQ ID NO: 10 - RNA, 21 nt, Artificial Sequence
  Synthetic
  misc_feature (20)..(21): modified with phosphorothioate
gaacugugugugagagguccu

SEQ ID NO: 11 - DNA, 21 bp, Artificial Sequence
  Synthetic
  misc_feature (20)..(21): modified with phosphorothioate
ccagguuuuuuuguuacuutt

SEQ ID NO: 12 - DNA, 21 bp, Artificial Sequence
  Synthetic
  misc_feature (20)..(21): modified with phosphorothioate
uuccucaaaucaauuaccatt

SEQ ID NO: 13 - DNA, 21 bp, Artificial Sequence
  Synthetic
  misc_feature (20)..(21): modified with phosphorothioate
ggaaggcucccuugauggatt

SEQ ID NO: 14 - DNA, 21 bp, Artificial Sequence
  Synthetic
  misc_feature (20)..(21): modified with phosphorothioate
gacacaguguguuugauuutt

SEQ ID NO: 15 - DNA, 21 bp, Artificial Sequence
  Synthetic
  misc_feature (20)..(21): modified with phosphorothioate
ugccaagccagauucucuutt

SEQ ID NO: 16 - DNA, 21 bp, Artificial Sequence
  Synthetic
  misc_feature (20)..(21): modified with phosphorothioate
cucaggaauucagugccuutt

SEQ ID NO: 17 - DNA, 21 bp, Artificial Sequence
  Synthetic
cuggacuuccagaagaacatt

SEQ ID NO: 18 - RNA, 21 nt, Artificial Sequence
  Synthetic
  misc_feature (1)..(1): modified with phosphorothioate
  misc_feature (20)..(20): modified with phosphorothioate
gucaucacacugaauaccaau

SEQ ID NO: 19 - RNA, 21 nt, Artificial Sequence
  Synthetic
  misc_feature (21)..(21): modified with phosphorothioate
gucaucacacugaauaccaau

SEQ ID NO: 20 - RNA, 18 nt, Artificial Sequence
  Synthetic
  misc_feature (1)..(1): modified with cycloalkyl
  misc_feature (3)..(3): modified with cycloalkyl
  misc_feature (11)..(11): modified with cycloalkyl
  misc_feature (18)..(18): modified with phosphorothioate
gacaucuucuuaaggacg

SEQ ID NO: 21 - RNA, 21 nt, Artificial Sequence
  Synthetic
  misc_feature (21)..(21): modified with 2′-O-methyl
gucaucacacugaauaccaau

SEQ ID NO: 22 - RNA, 21 nt, Artificial Sequence
  Synthetic
  misc_feature (21)..(21): modified with phosphorothioate
agguguauggcuucaacccug